(19) United States
(12) Patent Application Publication
(10) Pub. No.: US 2026/0199297 A1
(43) Pub. Date: Jul. 16, 2026

(54) FERROPTOSIS INDUCERS TO TREAT CANCER

(71) Applicant: The University of Toledo, Toledo, OH (US)

(72) Inventors: Viranga Tillekeratne, Toledo, OH (US); Samkeliso Dlamini, Toledo, OH (US); William Taylor, Toledo, OH (US)

(73) Assignee: The University of Toledo, Toledo, OH (US)

(21) Appl. No.: 19/135,522

(22) PCT Filed: Oct. 9, 2023
(86) PCT No.: PCT/US2023/034723

§ 371 Date: Jun. 4, 2025

Related U.S. Application Data
(60) Provisional application No. 63/431,431, filed on Dec. 9, 2022.

(51) Int. Cl. A61K 31/426; A61P 35/00; C07D 277/14; C07D 277/28; C07D 277/30; C07D 417/12; C07D 495/04

(52) U.S. Cl. CPC A61K 31/426; A61P 35/00; C07D 277/14; C07D 277/28; C07D 277/30; C07D 417/12; C07D 495/04

(57) ABSTRACT

Ferroptosis inducer compounds capable of killing cancer cells, along with methods of making and using the same, are described.

### RELATED APPLICATIONS

This application claims priority to U.S. Provisional Application No. 63/431,431 filed under 35 U.S.C. § 111(b) on Dec. 9, 2022, the disclosure of which is incorporated herein by reference in its entirety.

### STATEMENT REGARDING FEDERALLY SPONSORED RESEARCH

This invention was made with government support under Grant Number 1R15CA213185-01A1 awarded by the National Institutes of Health. The government has certain rights in this invention.

### BACKGROUND

Significant progress in cancer drug discovery and insight into the origins and mechanisms of cancer formation have yet to be translated into improved life span of cancer patients. Five years of cancer remission is the norm for determining the success of cancer treatment and it is still not achievable for some forms of cancer. According to 2021 statistics, cancer is the second leading cause of death in the United States. Numerous clinically used drugs are prone to multi-drug resistance, toxicity, and off-target effects. A formidable gap in cancer treatment strategies still exists, warranting the development of additional innovative cancer treatment tools.

A major setback in cancer therapy is the development of drug resistance by cancer cells and tumor metastasis. No effective drugs that prevent tumor metastasis are currently available, which is a major reason for the failure of current cancer treatment strategies. Combination therapies using two or more drugs acting concurrently by different mechanisms of action may be used to overcome drug resistance. Therefore, new cancer drugs acting by new mechanisms of action (preferably nonapoptotic) are needed to overcome these drawbacks.

### SUMMARY

Provided is a composition comprising Formula I:

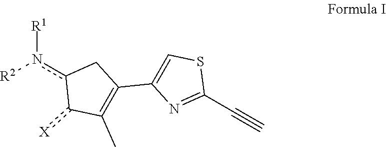

where dashed lines represent optional bonds, provided that nitrogen does not have more than three bonds and oxygen does not have more than two bonds; R¹ is OH, acyl, aryl, alkoxy, alkoxyalkyl, heteroaryl, aralkyl, or amidyl; R² is absent or hydrogen; and X is absent, OH, or OR³, wherein R³ is hydrogen, alkyl, alkoxy, or alkoxyalkyl. Also provided are salts, stereoisomers, racemates, solvates, hydrates, polymorphs, or prodrugs of Formula I.

In certain embodiments, R² is absent and X is OH. In certain embodiments, R² is hydrogen and X is OH. In certain embodiments, R² is absent and X is absent.

In certain embodiments, R² is absent, X is OR³, and R³ is alkyl, alkoxy, or alkoxyalkyl. In certain embodiments, R² is hydrogen, X is OR³, and R³ is alkyl, alkoxy, or alkoxyalkyl. In certain embodiments, R¹ is an ester with a terminal alkyne. In certain embodiments, R¹ is OR⁴, wherein R⁴ is aryl, aralkyl, or amidyl.

In certain embodiments, R¹ is OC═OR⁵, wherein R⁵ is alkyl. In particular embodiments, R⁵ is (CH₂)ₙCH₃, wherein n ranges from 1 to 10.

In certain embodiments, the composition comprises Formula II:

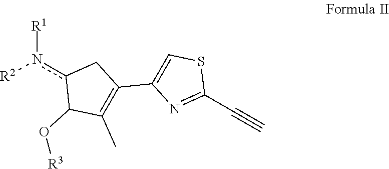

where dashed lines represent optional bonds, provided that nitrogen does not have more than three bonds and oxygen does not have more than two bonds; R¹ is OH, acyl, aryl, alkoxy, alkoxyalkyl, heteroaryl, aralkyl, or amidyl; R² is absent or hydrogen; and R³ is hydrogen, alkyl, alkoxy, or alkoxyalkyl.

In particular embodiments, R² is absent or hydrogen, and R³ is hydrogen. In particular embodiments, R² is absent or hydrogen, and R³ is alkyl. In particular embodiments, R² is absent or hydrogen, and R³ is alkoxy. In particular embodiments, R² is absent or hydrogen, and R³ is alkoxyalkyl. In particular embodiments, R² is hydrogen and R³ is hydrogen. In particular embodiments, R² is hydrogen and R³ is alkyl. In particular embodiments, R² is hydrogen and R³ is alkoxy. In particular embodiments, R² is hydrogen and R³ is alkoxyalkyl.

In certain embodiments, the composition comprises compound 14:

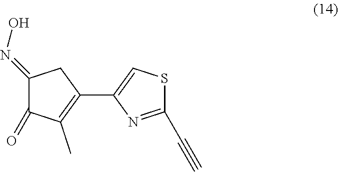 [SMILES: C#Cc1nc(C2=C(C)C(=O)/C(=N/O)C2)cs1]

In certain embodiments, the composition comprises compound 19:

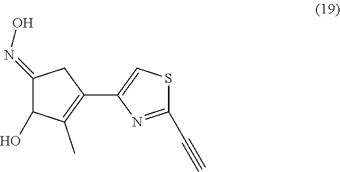 [SMILES: C#Cc1nc(C2=C(C)C(O)/C(=N/O)C2)cs1]

In certain embodiments, the composition comprises compound 18:

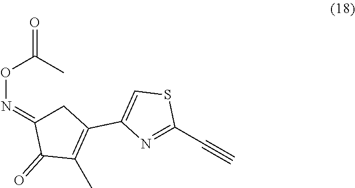 [SMILES: C#Cc1nc(C2=C(C)C(=O)/C(=N/OC(C)=O)C2)cs1]

In certain embodiments, the composition comprises compound 20a:

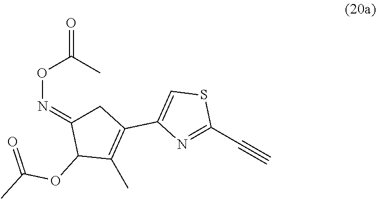 [SMILES: C#Cc1nc(C2=C(C)C(OC(C)=O)/C(=N/OC(C)=O)C2)cs1]

In certain embodiments, the composition comprises compound 20b:

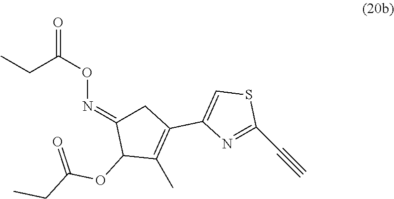 [SMILES: C#Cc1nc(C2=C(C)C(OC(=O)CC)/C(=N/OC(=O)CC)C2)cs1]

In certain embodiments, the composition comprises compound 20c:

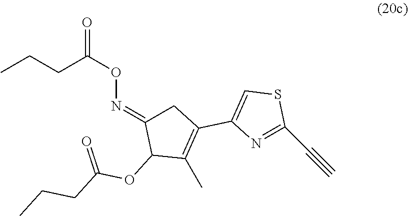 [SMILES: C#Cc1nc(C2=C(C)C(OC(=O)CCC)/C(=N/OC(=O)CCC)C2)cs1]

In certain embodiments, the composition comprises compound 20e:

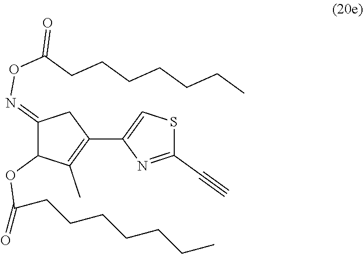

In certain embodiments, the composition comprises compound 21:

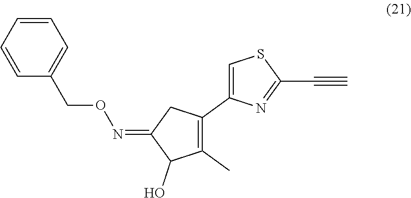 [SMILES: C#Cc1nc(C2=C(C)C(O)/C(=N/OCc3ccccc3)C2)cs1]

In certain embodiments, the composition comprises compound 23:

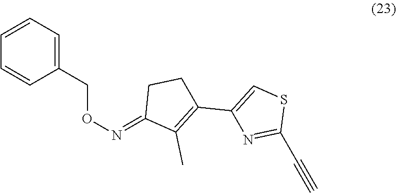 [SMILES: C#Cc1nc(C2=C(C)/C(=N/OCc3ccccc3)CC2)cs1]

In certain embodiments, the composition comprises PPc:

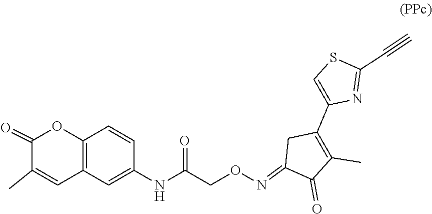 [SMILES: C#Cc1nc(C2=C(C)C(=O)/C(=N/OCC(=O)Nc3ccc4oc(=O)c(C)cc4c3)C2)cs1]

In certain embodiments, the composition comprises PPa:

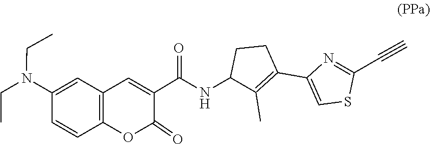 [SMILES: C#Cc1nc(C2=C(C)C(NC(=O)c3cc4cc(N(CC)CC)ccc4oc3=O)CC2)cs1]

In certain embodiments, the composition comprises PPb:

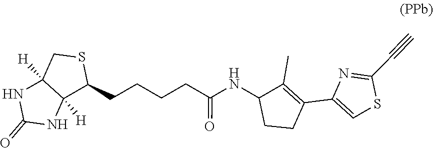 [SMILES: C#Cc1nc(C2=C(C)C(NC(=O)CCCC[C@@H]3SC[C@@H]4NC(=O)N[C@@H]43)CC2)cs1]

In certain embodiments, the composition comprises PPd:

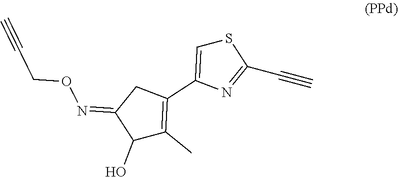 [SMILES: C#CCO/N=C1\CC(c2csc(C#C)n2)=C(C)C1O]

In certain embodiments, the composition comprises PPe:

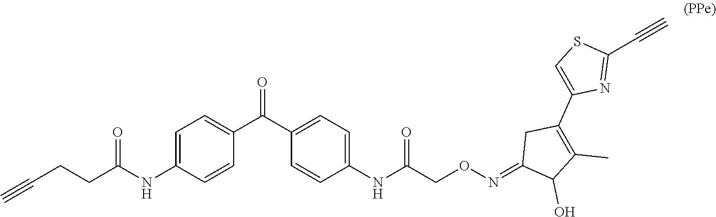 [SMILES: C#CCCC(=O)Nc1ccc(C(=O)c2ccc(NC(=O)CO/N=C3\CC(c4csc(C#C)n4)=C(C)C3O)cc2)cc1]

In certain embodiments, the composition comprises PPf:

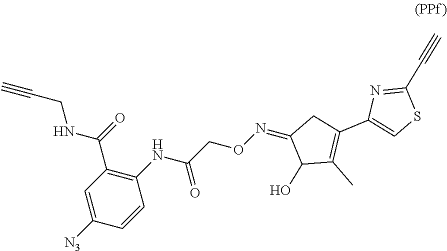 [SMILES: C#CCNC(=O)c1cc(N=[N+]=[N-])ccc1NC(=O)CO/N=C1/CC(c2csc(C#C)n2)=C(C)C1O]

Further provided is a method to kill cancer cells, the method comprising contacting cancer cells with an effective amount of a composition comprising Formula I to kill the cancer cells.

In certain embodiments, the cancer cells are mesenchymal non-small cell lung cancer cells. In certain embodiments, the cancer cells are fibrosarcoma cells. In certain embodiments, the cancer cells are osteosarcoma cells. In certain embodiments, the cancer cells are breast cancer cells. In certain embodiments, the composition comprises compound 20a and the cancer cells are leukemia cells, ovarian cancer cells, or renal cancer cells.

Further provided is a method of treating a cancer, the method comprising administering to a subject having a cancer an effective amount of a composition comprising Formula I to treat the cancer. In certain embodiments, the cancer is non-small cell lung cancer or breast cancer. In certain embodiments, the composition comprises compound 20a and the cancer is leukemia, ovarian cancer, or renal cancer.

Further provided is a method of inhibiting tumor metastasis in a subject, the method comprising administering an effective amount of a composition comprising Formula I to a subject having a tumor to inhibit tumor metastasis in the subject.

Further provided is a method of making a CETZOLE compound, the method comprising condensing an ethyl vinyl ketone with a thiazole aldehyde in the presence of a thiazolium salt catalyst to obtain a 1,4-addition product; cyclizing the 1,4-addition product to obtain a halo-ketone; reducing the halo-ketone to obtain an alcohol; coupling the alcohol with trimethylsilyl-acetylene to obtain a silylated ketone; desilylating the silylated ketone to obtain a ketone; and reducing the ketone to obtain a CETZOLE compound.

In certain embodiments, the CETZOLE compound is obtained in a racemic mixture. In certain embodiments, the CETZOLE compound is 3-(2-ethynylthiazol-4-yl)-2-methylcyclopent-2-en-1-ol.

In certain embodiments, the thiazolium salt catalyst is a thiazolium bromide. In certain embodiments, the coupling is Palladium-catalyzed Sonogashira coupling. In certain embodiments, the reduction of the ketone is with sodium borohydride in methanol. In certain embodiments, the condensing is conducted in anhydrous THF. In certain embodiments, the desilylating is conducted with potassium carbonate in methanol.

Further provided is a kit for synthesizing a CETZOLE compound, the kit comprising a first container housing one or both of an ethyl vinyl ketone and a thiazole aldehyde; a second container housing a thiazolium salt catalyst; and a third container housing trimethylsilyl-acetylene. In certain embodiments, the kit further comprises one or more solvents.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

PRIOR ART FIG. 1: Ferroptosis agents.

FIG. 2: Non-limiting example ferroptosis inducer compounds in accordance with the present disclosure.

FIGS. 3A-3B: Schemes depicting the conventional synthesis approach for CETZOLE (3-(2-ethynylthiazol-4-yl)-2-methylcyclopent-2-en-1-ol) 1 (FIG. 3A), and the improved synthesis approach for CETZOLE compounds in accordance with the present disclosure (FIG. 311).

FIG. 4: Scheme 3, showing the approach to functionalize CETZOLE ketone 7 α-carbon. (i) is the unsuccessful alkylation via enolate generated with LDA. (ii) is the synthesis of oxime 14 with tert-butyl nitrite.

FIG. 5: Scheme 4, depicting the synthesis of CETZOLE analogs and CETZOLE template analogs.

FIG. 6: Change in IC₅₀ values of analogs 14, 1, 21, 20a with change in structure.

FIG. 7: Mechanisitic pathway for CETZOLE's induction of ferroptosis death, showing Xc⁻, a possible target of CETZOLE. CETZOLE induced ferroptosis inhibition by Trolox, DFO, and BME.

FIG. 8: Methodology employed in the examples herein.

FIG. 9A: IC₅₀ values for compounds 20a, 20b, 20c, and 20e in comparison to CETZOLE (1) on the NCI-H522 cell line.

FIG. 911: IC₅₀ values for compounds 23 and 21 in comparison to CETZOLE (1) on the NCI-H522 cell line.

FIGS. 10A-10C: Results of live cell imaging. FIG. 10A shows a live cell time point picture montage of NCI-H522 cells treated with DMSO, CETZOLE 1, and CETZOLE analogs 20a and 23. FIG. 10B shows a cell survival analysis using Kaplan-Meier plots of NCI-H522 cells treated with DMSO, RSL3, CETZOLE 1, and analogs 20a, 23. FIG. 10C shows a three-day ferroptosis rescue assay of NCI-H522 cells treated with DMSO, CETZOLE 1, RSL3, and CETZOLE analogs 20a and 23 compared to co-treatment with liproxstatin-1.

FIGS. 11A-11E: Flow cytometry and BODIPY-C11 analysis of cellular ROS in NCI-H522 cells treated with CETZOLE analogs 20a (FIG. 11A), 20b (FIG. 11B), 20c (FIG. 11C), 29 (FIG. 11D), and CETZOLE 1 (FIG. 11E) for 3 hours.

FIG. 12: Selective cytotoxicity assay of analogs 20a-20c, and 23 relative to CETZOLE 1 and RSL3 (controls) at 1 μM concentration against NCI-H522 and HT-1080 cancer cell lines, and W138 and MEFs normal cell lines.

FIGS. 13A-13C: Growth inhibitory activity of CETZOLE 1 and analogues 20a and 23 in the National Cancer Institute 60 cell line assay. FIG. 13A shows the structures of the analogs tested. FIG. 13B shows a heat map of growth inhibitory activity of CETZOLE 1 and analogs 20a and 23 at 10 μM. FIG. 13C shows a heat map of GI₅₀ values of CETZOLE 1 and analogs 20a and 23 in the dose response assay.

FIGS. 14A-14D: FIG. 14A shows the synthesis of CETZOLE amide (PPa-PPb), fluorescent, propargyl (PPc-PPd), photo-reactive probes (PPe-PPf). CETZOLE oxime-ether probes and their corresponding negative controls (NPc-NPf) using the alkylation agents 28c-28f. FIG. 14A also shows the synthesis of alkylation agents 28c, 28e-28f. FIG. 14B shows the synthesis of coumarin amine acetyl bromide 28c. FIG. 14C shows the synthesis of benzophenone propargyl acetyl bromide 28e. FIG. 14D shows the synthesis of aryl azide propargyl acetyl bromide 28f.

FIG. 15: Structures of synthesized CETZOLE probes (PPa-PPf) and their corresponding negative controls (NPa-NPf). Colors represent: Blue=biorthogonal handle; Magenta=Fluorescent tag; Green=affinity tag; Orange: Photoaffinity group.

FIG. 16: Evaluation of ferroptosis-inducing character of CETZOLE probes. A three-day ferroptosis rescue assay of NCI-H522 cells treated with DMSO, 1, RSL3, and CETZOLE analogs, and liproxstatin-1 co-treatment, showing significant ferroptosis inducing probes.

FIGS. 17A-17E: Cellular localization of CETZOLE probes. FIG. 17A shows a workflow diagram of live cell imaging of HeLa cells treated with DMSO, PPd, and the corresponding negative probe NPd. Cells were incubated with the respective agent, permeabilized, subjected to TAMRA-PEG3 azide ligation, and viewed under microscope. FIG. 17B shows the probes employed in bioimaging. FIG. 17C shows live cell bioimages of cells treated with DMSO, PPd, and NPd. FIG. 17D shows a single cell image of PPd-treated cells showing fluorescing intracellular cell organelles. FIG. 17E shows a single cell image of PPd-treated cells showing fluorescing chromosomes.

FIGS. 18A-18E: FIG. 18A shows a workflow diagram for dose-dependent competitive binding assay of PPd and negative control NPd with increasing concentrations of CETZOLE 1. FIG. 18B shows a binding assay screening of NCI-H522, HT-1080, and MDA-MB231 cancer cell lines. FIG. 18C shows a competitive binding assay of PPd against increasing concentrations (5-40 μM) of CETZOLE 1. FIG. 18D shows a competitive binding study against selected known ferroptosis inducers (CETZOLE 1, RSL3, erastin, ML210, ML160, and SSZ). FIG. 18E shows the structure of TAMRA-PEG-3-azide used for ligation.

FIGS. 19A-19F: FIG. 19A shows a workflow diagram for protein target pull down assay with of PPd and corresponding negative probe NPd. FIG. 19B shows a fluorescence gel of the pull-down assay with PPd and corresponding negative probe NPd with target band highlighted with red asterisk. FIG. 19C shows a Venn diagram showing 489 proteins unique to PPd, 250 proteins unique to NPd, and common proteins from both treatments (911 proteins). FIG. 19D shows a Venn diagram showing highly enriched proteins and sub-Venn diagram showing enriched anti-oxidation related proteins. FIG. 19E shows i. CBS, ii. GPX4, and iii. PRDX4 western blot target protein validation with antibodies. FIG. 19F shows the biotin-TAMRA-PEG-3-azide used for ligation.

FIGS. 20A-20B: FIG. 20A is a Venn diagram showing highly enriched proteins and sub-Venn diagram showing enriched anti-oxidation related proteins. FIG. 20B shows i. NEDD4L, and ii. GST01 western blot target protein validation with antibodies.

FIG. 21: Results of NCI-60 cell line assay at 10 nM with CETZOLE (1) and compounds 19, 20a, 23, and 18.

FIGS. 22A-22B: ¹H NMR (FIG. 22A) and ¹³C NMR (FIG. 22B) spectra of 1-(2-bromothiazol-4-yl)hexane-1,4-dione (12).

FIGS. 23A-23B: ¹H NMR (FIG. 23A) and ¹³C NMR (FIG. 23B) spectra of 2-methyl-3-(2-((trimethylsilyl)ethynyl)thiazol-4-yl)cyclopent-2-en-1-one (6).

FIGS. 24A-24B: ¹H NMR (FIG. 24A) and ¹³C NMR (FIG. 24B) spectra of 3-(2-ethynylthiazol-4-yl)-2-methylcyclopent-2-en-1-one (7).

FIGS. 25A-25B: ¹H NMR (FIG. 25A) and ¹³C NMR (FIG. 25B) spectra of 3-(2-ethynylthiazol-4-yl)-2-methylcyclopent-2-en-1-ol (1).

FIGS. 26A-26B: ¹H NMR (FIG. 26A) and ¹³C NMR (FIG. 26B) spectra of 3-(2-bromothiazol-4-yl)-2-methylcyclopent-2-en-1-ol (13).

FIGS. 27A-27B: ¹H NMR (FIG. 27A) and ¹³C NMR (FIG. 27B) spectra of (E)-3-(2-ethynylthiazol-4-yl)-5-(hydroxyimino)-2-methylcyclopent-2-en-1-one (14).

FIGS. 28A-28B: ¹H NMR (FIG. 28A) and ¹³C NMR (FIG. 28B) spectra of (E)-3-(2-bromothiazol-4-yl)-5-(hydroxyimino)-2-methylcyclopent-2-en-1-one (26).

FIGS. 29A-29B: ¹H NMR (FIG. 29A) and ¹³C NMR (FIG. 29B) spectra of (E)-4-(2-ethynylthiazol-4-yl)-2-hydroxy-3-methylcyclopent-3-en-1-one oxime (19).

FIGS. 30A-30B: ¹H NMR (FIG. 30A) and ¹³C NMR (FIG. 30B) spectra of (E)-4-(2-bromothiazol-4-yl)-2-hydroxy-3-methylcyclopent-3-en-1-one oxime (27).

FIGS. 31A-31B: ¹H NMR (FIG. 31A) and ¹³C NMR (FIG. 31B) spectra of (E)-5-(acetoxyimino)-3-(2-ethynylthiazol-4-yl)-2-methylcyclopent-2-en-1-yl acetate (20a).

FIGS. 32A-32B: ¹H NMR (FIG. 32A) and ¹³C NMR (FIG. 32B) spectra of (E)-3-(2-ethynylthiazol-4-yl)-2-methyl-5-((propionyloxy)imino)cyclopent-2-en-1-yl propionate (20b).

FIGS. 33A-33B: ¹H NMR (FIG. 33A) and ¹³C NMR (FIG. 33B) spectra of (E)-5-((butyryloxy)imino)-3-(2-ethynylthiazol-4-yl)-2-methylcyclopent-2-en-1-yl butyrate (20c).

FIGS. 34A-34B: ¹H NMR (FIG. 34A) and ¹³C NMR (FIG. 34B) spectra of E)-3-(2-ethynylthiazol-4-yl)-2-methyl-5-((octanoyloxy)imino)cyclopent-2-en-1-yl octanoate (20e).

FIGS. 35A-35B: ¹H NMR (FIG. 35A) and ¹³C NMR (FIG. 35B) spectra of (E)-3-(2-ethynylthiazol-4-yl)-2-methylcyclopent-2-en-1-one O-benzyl oxime (23).

FIGS. 36A-36B: ¹H NMR (FIG. 36A) and ¹³C NMR (FIG. 36B) spectra of (Z)-4-(2-ethynylthiazol-4-yl)-2-hydroxy-3-methylcyclopent-3-en-1-one O-benzyl oxime (21).

FIGS. 37A-37B: ¹H NMR (FIG. 37A) and ¹³C NMR (FIG. 37B) spectra of 3-(2-bromothiazol-4-yl)-2-methylcyclopent-2-en-1-amine (24).

FIGS. 38A-38B: ¹H NMR (FIG. 38A) and ¹³C NMR (FIG. 38B) spectra of N-(3-(2-bromothiazol-4-yl)-2-methylcyclopent-2-en-1-yl)-7-(diethylamino)-2-oxo-2H-chromene-3-carboxamide (NPa).

FIGS. 39A-39B: ¹H NMR (FIG. 39A) and ¹³C NMR (FIG. 39B) spectra of 7-(diethylamino)-N-(3-(2-ethynylthiazol-4-yl)-2-methylcyclopent-2-en-1-yl)-2-oxo-2H-chromene-3-carboxamide (PPa).

FIGS. 40A-40B: ¹H NMR (FIG. 40A) and ¹³C NMR (FIG. 40B) spectra of N-(3-(2-bromothiazol-4-yl)-2-methylcyclopent-2-en-1-yl)-5-((3aS,4S,6aR)-2-oxohexahydro-1H-thieno[3,4-d]imidazol-4-yl)pentanamide (NPb).

FIGS. 41A-41B: ¹H NMR (FIG. 41A) and ¹³C NMR (FIG. 41B) spectra of N-(3-(2-ethynylthiazol-4-yl)-2-methylcyclopent-2-en-1-yl)-5-((3aS,4S,6aR)-2-oxohexahydro-1H-thieno[3,4-d]imidazol-4-yl)pentanamide (PPb).

FIGS. 42A-42B: ¹H NMR (FIG. 42A) and ¹³C NMR (FIG. 42B) spectra of N-(4-(4-aminobenzoyl)phenyl)pent-4-ynamide (33).

FIGS. 43A-43B: ¹H NMR (FIG. 43A) and ¹³C NMR (FIG. 43B) spectra of 2-bromo-N-(3-methyl-2-oxo-2H-chromen-6-yl)acetamide (28c).

FIGS. 44A-44B: ¹H NMR (FIG. 44A) and ¹³C NMR (FIG. 44B) spectra of N-(4-(4-(2-bromoacetamido)benzoyl)phenyl)pent-4-ynamide (28e).

FIGS. 45A-45B: ¹H NMR (FIG. 45A) and ¹³C NMR (FIG. 45B) spectra of 4-(2-ethynylthiazol-4-yl)-2-hydroxy-3-methylcyclopent-3-en-1-one O-prop-2-yn-1-yl oxime (PPd).

FIGS. 46A-46B: ¹H NMR (FIG. 46A) and ¹³C NMR (FIG. 46B) spectra of 4-(2-bromothiazol-4-yl)-2-hydroxy-3-methylcyclopent-3-en-1-one O-prop-2-yn-1-yl oxime (NPd).

FIGS. 47A-47B: ¹H NMR (FIG. 47A) and ¹³C NMR (FIG. 47B) spectra of (E)-2-(((4-(2-ethynylthiazol-4-yl)-3-methyl-2-oxocyclopent-3-en-1-ylidene)amino)oxy)-N-(3-methyl-2-oxo-2H-chromen-6-yl)acetamide (PPc).

FIGS. 48A-48B: ¹H NMR (FIG. 48A) and ¹³C NMR (FIG. 48B) spectra of (E)-2-(((4-(2-bromothiazol-4-yl)-2-hydroxy-3-methylcyclopent-3-en-1-ylidene)amino)oxy)-N-(3-methyl-2-oxo-2H-chromen-6-yl)acetamide (NPc).

FIGS. 49A-49B: ¹H NMR (FIG. 49A) and ¹³C NMR (FIG. 49B) spectra of (E)-N-(4-(4-(2-(((4-(2-ethynylthiazol-4-yl)-2-hydroxy-3-methylcyclopent-3-en-1-ylidene)amino)oxy)acetamido)benzoyl)phenyl)pent-4-ynamide (PPe).

FIGS. 50A-50B: ¹H NMR (FIG. 50A) and ¹³C NMR (FIG. 50B) spectra of (E)-N-(4-(4-(2-(((4-(2-bromothiazol-4-yl)-2-hydroxy-3-methylcyclopent-3-en-1-ylidene)amino)oxy)acetamido)benzoyl)phenyl)pent-4-ynamide (NPe).

FIGS. 51A-51B: ¹H NMR (FIG. 51A) and ¹³C NMR (FIG. 51B) spectra of methyl 5-bromo-2-((tert-butoxycarbonyl)amino)benzoate (35).

FIGS. 52A-52B: ¹H NMR (FIG. 52A) and ¹³C NMR (FIG. 52B) spectra of methyl 5-azido-2-((tert-butoxycarbonyl)amino)benzoate (36).

FIGS. 53A-53B: ¹H NMR (FIG. 53A) and ¹³C NMR (FIG. 53B) spectra of 5-azido-2-((tert-butoxycarbonyl)amino)benzoic acid (37).

FIGS. 54A-54B: ¹H NMR (FIG. 54A) and ¹³C NMR (FIG. 54B) spectra of tert-butyl (4-azido-2-(prop-2-yn-1-ylcarbamoyl)phenyl)carbamate (39).

FIGS. 55A-55B: ¹H NMR (FIG. 55A) and ¹³C NMR (FIG. 55B) spectra of 2-amino-5-azido-N-(prop-2-yn-1-yl)benzamide (40).

FIGS. 56A-56B: ¹H NMR (FIG. 56A) and ¹³C NMR (FIG. 56B) spectra of 5-azido-2-(2-bromoacetamido)-N-(prop-2-yn-1-yl)benzamide (28f).

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Provided herein is a class of small molecules that kill cancer cells by ferroptosis, which is a nonapoptotic cell death mechanism. Described in 2012, ferroptosis is a non-apoptotic cell death mechanism impelled by unrestrained accumulation of iron-dependent cellular reactive oxygen species (ROS), leading to membrane lipid peroxidation resulting from intra-cellular antioxidant depletion. Cytological disruption involving lysis of the plasma membrane, mitochondrial membrane, and the mitochondrial cristae in the presence of ROS distinguish ferroptosis from autophagic, apoptotic, and necrotic cell death. Discovered through high throughput screening, erastin and RSL3 (Ras-selective lethal 3) are the earliest described ferroptosis agents that selectively target a family of oncogenic mutant HRAS^{V12} tumor cells (PRIOR ART FIG. 1). Erastin administration depletes intra-cellular antioxidants by inhibiting mitochondrial VDAC2/3 and disulfide linked heterodimeric cell membrane antiporter X_{c}⁻, disrupting cysteine-glutamate homeostasis. It is termed a type I ferroptotic agent. This negates the formation of the essential cellular anti-oxidants like glutathione, allowing ROS and toxic glutamate accumulation. Restricted glutathione production indirectly inhibits selenocysteine hydro-peroxidase GPX4, which catalyzes the oxidization of reduced glutathione (GSH), detoxifying lipid hydroperoxides to nontoxic lipid alcohols. RSL3 is a GPX4 inhibitor. GPX4 is a seleno-protein that lacks a drug-like binding pocket. It can be targeted by type II inhibitors with an electrophilic war head that covalently engages the selenocysteine residue. RSL3 alkylates GPX4 via a covalently reacting chloro-acetamide electrophile, but is prone to promiscuity and poor selectivity. Unlike erastin, RSL3 does not target X_{c}⁻ antiporter, yet both are RAS lethal molecules that induce iron-dependent accumulation of ROS in the presence of cellular iron by targeting either upstream or downstream anti-oxidation related proteins.

Non-apoptotic cell death due to continuous ROS accumulation is negated by iron chelators such as deferoxamine or radical trapping anti-oxidants such as ferrostatin-1 and liproxstatin-1. Clinical application of ferroptosis agents/drugs has been elusive due to off-target effects leading to drug toxicity. Improved understanding of protein targets and the mechanism of action of ferroptosis agents can contribute to their successful clinical application. Thus, there is a need for novel nontoxic and highly selective ferroptosis agents. Being a relatively recently discovered cell death mechanism, there are no ferroptosis inducers currently in clinical use. However, in some embodiments, the ferroptosis inducer compounds described herein kill selected cancer cells at nanomolar to low micromolar concentrations. As shown in the examples herein, cancer stem cell-like mesenchymal type cancer cells are particularly sensitive to these ferroptosis inducer compounds and, therefore, the ferroptosis inducer compounds have the ability to prevent tumor metastasis.

U.S. Pat. Nos. 9,862,692 and 10,138,216, which are incorporated herein by reference, disclose the ferroptosis inducer CETZOLE (1), which has the following structure:

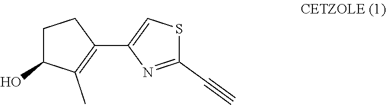

The compounds of the present disclosure are more potent than CETZOLE (1) and the other molecules disclosed in U.S. Pat. Nos. 9,862,692 and 10,138,216. Furthermore, the method of synthesis of the ferroptosis inducer compounds described herein is an improvement relative to the synthesis methods disclosed in U.S. Pat. Nos. 9,862,692 and 10,138,216, as described in more detail below.

In general, the ferroptosis inducer compounds in accordance with the present disclosure have the following general structural Formula I:

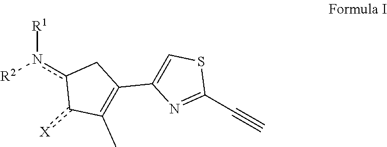

where dashed lines represent optional bonds, provided that nitrogen does not have more than three bonds and oxygen does not have more than two bonds; R¹ is OH, acyl, aryl, alkoxy, alkoxyalkyl, heteroaryl, aralkyl, or amidyl; R² is absent or hydrogen; and X is absent, OH, or OR³, wherein R³ is hydrogen, alkyl, alkoxy, or alkoxyalkyl. (Where X is OH or OR³, only one of the bonds leading to X represented by the dashed lines is present.) In some embodiments, the ferroptosis inducer compounds have the following general structural Formula II:

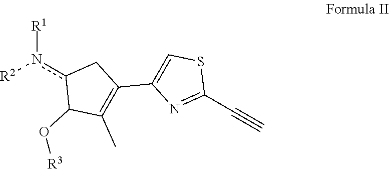

where dashed lines represent optional bonds, provided that nitrogen does not have more than three bonds and oxygen does not have more than two bonds; R¹ is OH, acyl, aryl, alkoxy, alkoxyalkyl, heteroaryl, aralkyl, or amidyl; R² is absent or hydrogen; and R³ is hydrogen, alkyl, alkoxy, or alkoxyalkyl.

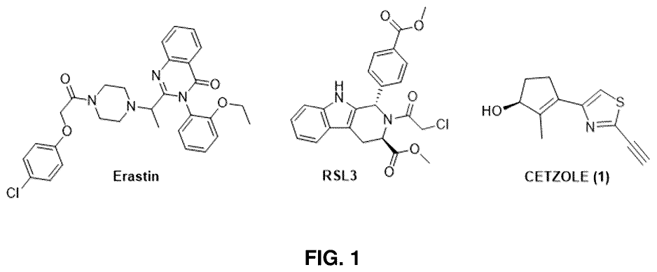

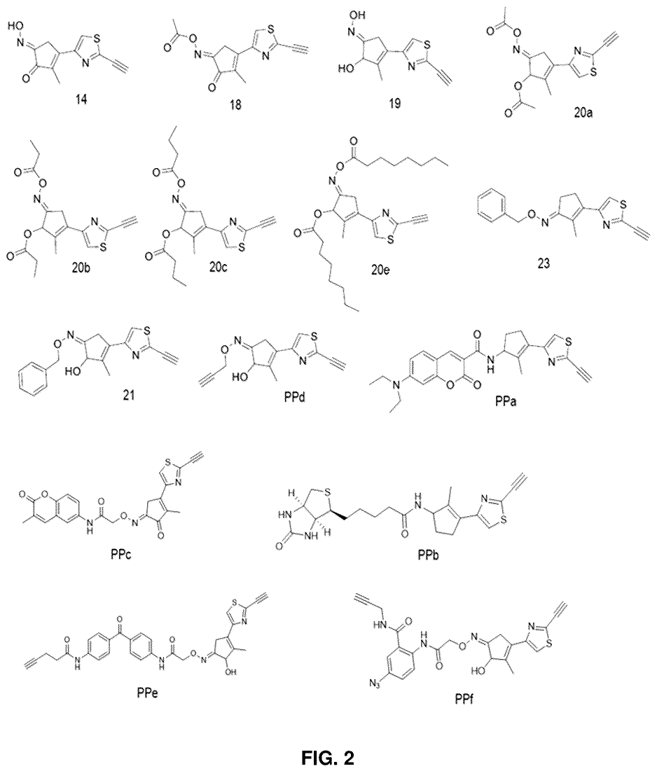

Some non-limiting example ferroptosis inducer compounds are depicted in FIG. 2. As shown in the examples herein, these compounds are effective at killing cancer cells through ferroptosis. The ferroptosis inducer compounds are anticancer agents that can be used alone or in combination with other drugs to treat cancer and prevent tumor metastasis. Advantageously, the ferroptosis inducer compounds are particularly effective on mesenchymal cells similar to cancer stem cells which are responsible for tumor metastasis, indicating the compounds are useful in preventing tumor metastasis, which is a major reason for the failure of current cancer treatment strategies.

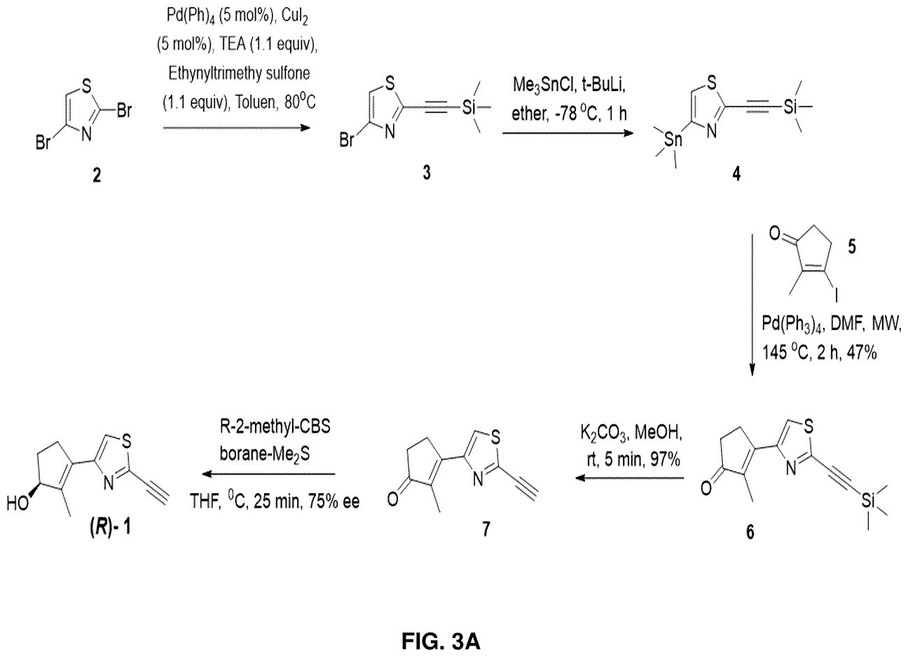

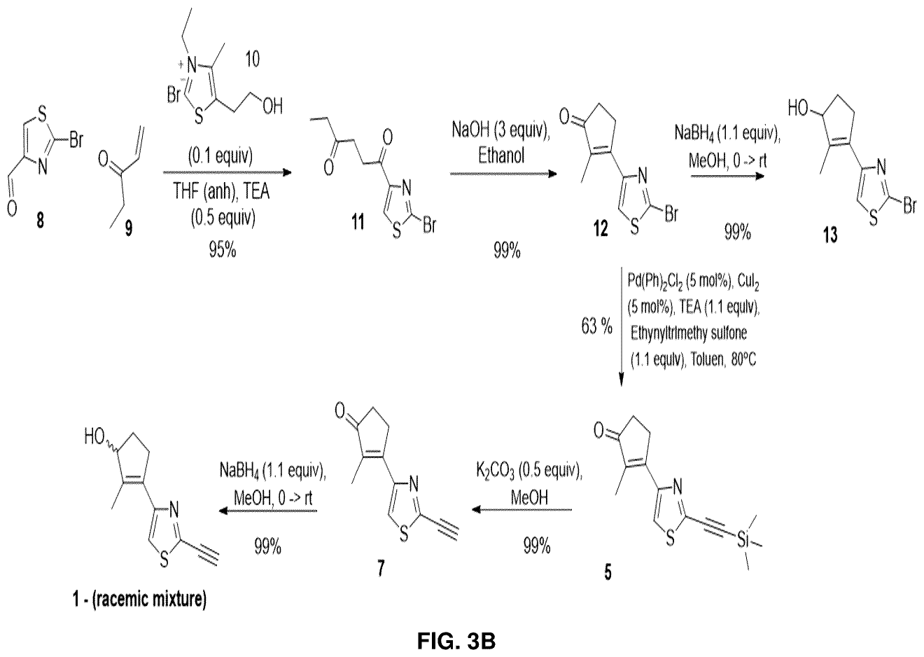

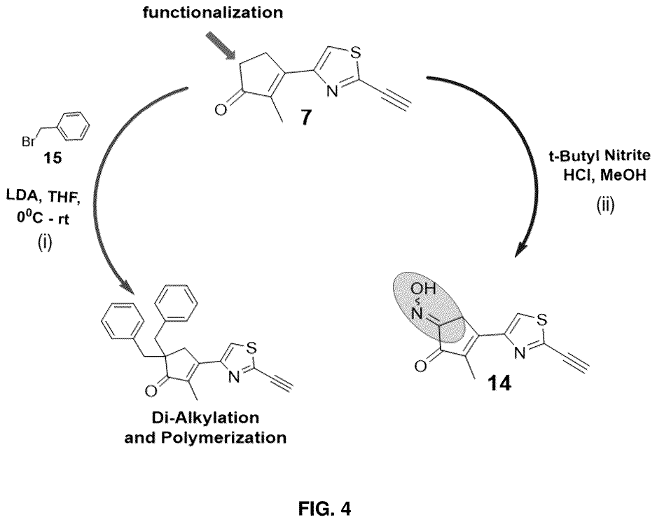

The ferroptosis inducer compounds may be synthesized according to the schemes depicted in FIGS. 3B-6. Notably, the method of synthesizing a CETZOLE-containing compound depicted in FIG. 3B is an improvement over the method for preparing CETZOLE compounds disclosed in U.S. Pat. Nos. 9,862,692 and 10,138,216, because it utilizes milder reaction conditions and avoids the use of toxic tin reagents.

As seen in FIG. 3B, a CETZOLE compound, such as, but not limited to, the ferroptosis inducer compounds described herein, and also including CETZOLE (1) itself, can be synthesized by first condensing an ethyl vinyl ketone with a thiazole aldehyde in the presence of a thiazolium salt catalyst to obtain a 1,4-addition product. The condensing may be conducted, for example, in anhydrous THF. The thiazolium salt catalyst may be, for example, thiazolium bromide. However, other thiazolium salt catalysts are possible and encompassed within the scope of the present disclosure. Then, the 1,4-addition product can be cyclized to obtain a halo-ketone. The halo-ketone can be reduced to obtain an alcohol. The reduction of the ketone may be conducted, for example, using sodium borohydride in methanol. The alcohol can be coupled with trimethylsilyl-acetylene to obtain a silylated ketone. The coupling can be Palladium-catalyzed Sonogashira coupling. The silylated ketone can be desilylated to obtain a ketone. The desilylating may be conducted, for example, with potassium carbonate in methanol. The ketone can be reduced to obtain a CETZOLE compound. Advantageously, the CETZOLE is obtained in a racemic mixture.

Pharmaceutical compositions of the present disclosure may comprise an effective amount of a ferroptotosis inducer compound (an “active compound” or “active ingredient”), optionally with additional agents, dissolved or dispersed in a pharmaceutically acceptable carrier. The preparation of a pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid or aerosol form, and whether it need to be sterile for such routes of administration as injection. Compositions disclosed herein can be administered intravenously, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a composition herein and/or additional agent is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally (U.S. Pat. Nos. 6,753,514, 6,613,308, 5,466,468, 5,543,158; 5,641,515, and 5,399,363 are each specifically incorporated herein by reference in their entirety).

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In some cases, the form must be sterile and must be fluid to the extent that easy injectability exists. It should be stable under the conditions of manufacture and storage and should be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption such as, for example, aluminum monostearate or gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Sterile injectable solutions are prepared by incorporating the compositions in the required amount in the appropriate solvent with various other ingredients enumerated above, as required, followed by filtered sterilization. Generally, dispersions are prepared by incorporating the various sterilized compositions into a sterile vehicle which contains the basic dispersion medium and the required other ingredients from those enumerated above. In the case of sterile powders for the preparation of sterile injectable solutions, some methods of preparation are vacuum-drying and freeze-drying techniques which yield a powder of the active ingredient plus any additional desired ingredient from a previously sterile-filtered solution thereof. A powdered composition is combined with a liquid carrier such as, but not limited to, water or a saline solution, with or without a stabilizing agent.

In other embodiments, the compositions may be formulated for administration via various miscellaneous routes, for example, topical (i.e., transdermal) administration, mucosal administration (intranasal, vaginal, etc.) and/or via inhalation.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and luarocapram. Possible bases for compositions for topical application include polyethylene glycol, lanolin, cold cream, and petrolatum, as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroetheylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation consists of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight, and the severity and response of the symptoms.

In particular embodiments, the compounds and compositions described herein are useful for treating cancers or killing cancer cells. As described herein, the compounds and compositions herein can be used in combination therapies. That is, the compounds and compositions can be administered concurrently with, prior to, or subsequent to one or more other desired therapeutic or medical procedures or drugs. The particular combination of therapies and procedures in the combination regimen will take into account compatibility of the therapies and/or procedures and the desired therapeutic effect to be achieved. Combination therapies include sequential, simultaneous, and separate administration of the active compound in a way that the therapeutic effects of the first administered procedure or drug is not entirely disappeared when the subsequent procedure or drug is administered.

In some embodiments, the ferroptosis inducer compound is part of a combination therapy with a chemotherapeutic agent. Suitable chemotherapeutic agents include, but are not limited to: taxane compounds, such as paclitaxel; platinum coordination compounds; topoisomerase I inhibitors, such as camptothecin compounds; topoisomerase II inhibitors, such as anti-tumor podophyllotoxin derivatives; anti-tumor vinca alkaloids; anti-tumor nucleoside derivatives; alkylating agents; anti-tumor anthracycline derivatives; HER2 antibodies; estrogen receptor antagonists or selective estrogen receptor modulators; aromatase inhibitors; differentiating agents, such as retinoids, and retinoic acid metabolism blocking agents (RAMBA); DNA methyl transferase inhibitors; kinase inhibitors; farnesyltransferase inhibitors; HDAC inhibitors, or other inhibitors of the ubiquitin-proteasome pathway; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide, and trimethylomelamine; acetogenins; camptothecins, such as the synthetic analog topotecan; cryptophycins; nitrogen mustards, such as chlorambucil; nitrosoureas; bisphosphonates; mitomycins; epothilones; maytansinoids; trichothecenes; retinoids, such as retinoic acid; pharmaceutically acceptable salts, acids and derivatives of any of the above; and combinations thereof. Non-limiting examples of specific chemotherapeutic agents include erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethyl-ethanamine, NOLVADEX®, ISTUBAL®, VALODEX®), doxorubicin (ADRIAMYCIN®), Akti-1/2, HPPD, rapamycin, lapatinib (TYKERB®, Glaxo SmithKline), oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (MEK inhibitor, Exelixis, WO 2007/044515), ARRY-886 (MEK inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), ABT-869 (multi-targeted inhibitor of VEGF and PDGF family receptor tyrosine kinases, Abbott Laboratories and Genentech), ABT-263 (Bcl-2/Bcl-xL inhibitor, Abbott Laboratories and Genentech), PTK787/ZK 222584 (Novartis), fulvestrant (FASLODEX®, AstraZeneca), leucovorin (folinic acid), lonafamib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifamib (ZARNESTRA™, Johnson & Johnson), capecitabine (XELODA®, Roche), ABRAXANET™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thioTepa and cyclosphosphamide (CYTOXAN®, NEOSAR®), bullatacin, bullatacinone, bryostatin, callystatin, CC-1065 (including its adozelesin, carzelesin and bizelesin synthetic analogs), cryptophycin 1, cryptophycin 8, dolastatin, duocarmycin (including the synthetic analogs, KW-2189 and CB1-TM1), leutherobin, pancratistatin, sarcodictyin, spongistatin, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, clodronate, esperamicin, neocarzinostatin chromophore and related chromoprotein enediyne antibiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, Oreg.), razoxane, rhizoxin, sizofuran, spirogermanium, tenuazonic acid, triaziquone, 2,2′,2″-trichlorotriethylamine, T-2 toxin, verracurin A, roridin A, anguidine, urethane, vindesine, dacarbazine, mannomustine, mitobronitol, mitolactol, pipobroman, gacytosine, arabinoside (“Ara-C”), cyclophosphamide, thioTepa, 6-thioguanine, mercaptopurine, vinblastine, etoposide (VP-16), ifosfamide, mitoxantrone, vincristine, vinorelbine (NAVELBINE®), novantrone, teniposide, edatrexate, daunomycin, aminopterin, ibandronate, CPT-11, topoisomerase inhibitor RFS 2000, and difluoromethylomithine (DMFO), paclitaxel, 5-fluorouracil, abraxane (paclitaxel albumin-stabilized nanoparticle formulation), afinitor (everolimus), erlotinib hydrochloride, everolimus, gemcitabine hydrochloride, oxaliplatin (eloxatin), capecitabine (xeloda), cisplatin, irinotecan (camptosar), colinic acid (leucovorin), folfox (folinic acid, 5-fluorouracil, and oxaliplatin), folfirinox (folinic acid, 5-fluorouracil, irinotecan, and oxaliplatin), nab-paclitaxel with gemcitabine, metformin, digoxin, and simvastatin.

In some embodiments, the ferroptosis inducer compound is part of a combination therapy with an immunotherapeutic agent. Non-limiting examples of immunotherapeutic agents include nivolumab, pembrolizumab, rituximab, durvalumab, cemiplimab, and combinations thereof.

In some embodiments, the ferroptosis inducer compound is part of a combination therapy with a hormonal therapeutic agent. Non-limiting examples of hormonal therapeutic agents include anastrozole, exemestane, letrozole, tamoxifen, raloxifene, fulvestrant, toremifene, gosrelin, leuprolide, triptorelin, apalutamide, enzalutamide, darolutamide, bicalutamide, flutamide, nilutamide, abiraterone, ketoconazole, degarelix, medroxyprogesterone acetate, megestrol acetate, mitotane, and combinations thereof.

The compositions and methods described herein may also be made available via a kit containing one or more key components. A non-limiting example of such a kit comprises a thiazolium salt catalyst and one or both of an ethyl vinyl ketone and a thiazole aldehyde in separate containers, where the containers may or may not be present in a combined configuration. Many other kits are possible, such as kits that further include one or more solvents. The kits typically further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

Advantageously, the ferroptosis inducer compounds described herein are particularly effective on mesenchymal cells, similar to cancer stem cells which are responsible for tumor metastasis. The ferroptosis inducer compounds may be useful in preventing tumor metastasis, either alone or in combination with other drugs. The ferroptosis inducer compounds kill cancer cells by a nonapoptotic mechanism, with the ability to overcome drug resistance, and are effective on cancer stem cells, providing the ability prevent cancer metastasis. Furthermore, the ferroptosis inducer compounds can be prepared by an efficient synthesis amenable to large-scale process chemistry.

### EXAMPLES

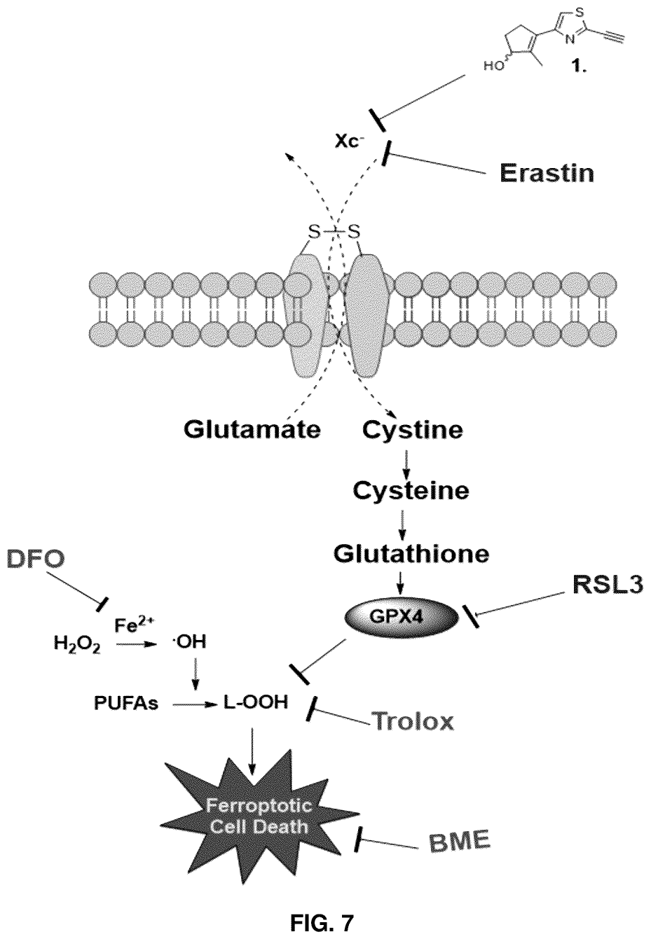

Ferroptosis cell death is controlled by a complex nonlinear network of genes and an in-depth scrutiny is necessary to understand this network. It has been shown that p53 enhances ferroptosis. Surprisingly, the p53 targets gene p21 and inhibits ferroptosis, indicating that p53 has other pro-ferroptotic target genes that predominate. In cell cycle regulation, p21 is an inhibitor of cyclin-dependent kinases (CDKs). CDKs generally drive progression through interphase by phosphorylating RB proteins, thereby releasing active E2F transcription factor which induces cell cycle genes. CDKs, RB, and E2F all regulate ferroptosis but not in a linear pathway as observed with cell cycle regulation. Therefore, these proteins likely have multiple, yet-to-be-discovered ferroptosis targets. This attests to the complexity of the ferroptosis mechanism. The ferroptosis inducer CETZOLE 1 (PRIOR ART FIG. 1) was used in these examples to uncover the complexity underlining the mechanism of action of ferroptosis. CETZOLE 1 consists of a 4-cyclopentenyl-2-ethynylthiazole scaffold (therefore, referred to as CETZOLEs). CETZOLE 1 induces ferroptosis more selectively in mesenchymal cancer cells such as HOP-62, NCI-H522, UACC-62, and A498, as opposed to epithelial cancer cells such as HCT-116, HeLa, and MCF7, which are less sensitive. Co-administration of iron chelators such as ciclopirox olamine and hydroxyurea diminished CETZOLE's potency, as summarized in FIG. 7 Similarly, free radical scavengers Trolox and butylated hydroxy anisole also negate CETZOLE cytotoxicity. β-mercapto-ethanol cotreatment blocks CETZOLE-induced cell death, similarly to erastin. In contrast, co-treatment with ferric citrate has the opposite effect. Furthermore, there is a significant reduction in glutathione levels after CETZOLE treatment. Conversely, BODIPY-C11 co-treated cells analyzed by flow cytometry show time-dependent cellular ROS accumulation. Similar to erastin, these are classic hallmarks of ferroptosis. Without wishing to be bound by theory, it is believed that CETZOLE 1 is a X_{c}⁻ inhibitor, with additional ferroptosis-related protein targets. This may account for the differences in the sensitivity of different cell lines to CETZOLE 1 and erastin. For example, HCT-116 colon cancer cell line is sensitive to erastin at low drug concentrations. CETZOLE 1, on the other hand, is less toxic to HCT-116 cells, as well as to normal cell lines.

### CETZOLE and CETZOLE Analogs as Highly Potent Ferroptotic Agents: Their Target Protein Identification Using Covalent/Affinity Probes

CETZOLEs represent a class of ferroptosis inducers. A structure-activity relationship study of these molecules led to the discovery of highly potent CETZOLE analogues. The analogues were confirmed to be ferroptosis agents that induce cell death through ROS accumulation by cell rescue and flow cytometry experiments. They are more cytotoxic to cancer cells over normal cells. Target protein identification studies using molecular probes led to the identification of cystathionine β-synthase, peroxiredoxins, ADP/ATP Carriers (SLC25A5), glucose dehydrogenase, VDAC, NEDD4L, GST01, and thioredoxin as possible targets of CETZOLEs. These proteins as well as GPX4 were validated through western blotting and are known to be associated with cellular antioxidant pathways.

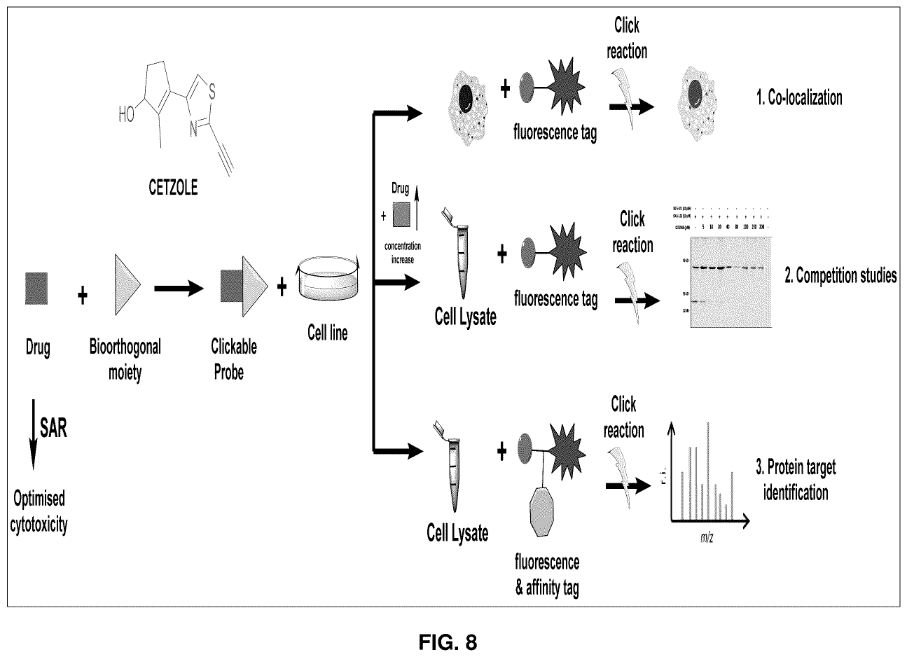

In these examples, the design and synthesis of CETZOLE analogs and covalent/affinity probes for protein target identification are described. As a prerequisite to developing chemical probes, a structure-activity relationship (SAR) study of CETZOLE was performed to evaluate the effect of structure modifications on cytotoxicity. Changes to the synthesis route and further functionalization of CETZOLE at the homo-allylic position through oxime formation permitted the synthesis of a small library of highly cytotoxic CETZOLE analogs, including prodrug variants. FIG. 8 depicts the methodology utilized in these examples.

FIG. 3A depicts the previous synthesis approach for CETZOLE (3-(2-ethynylthiazol-4-yl)-2-methylcyclopent-2-en-1-ol) 1, and FIG. 3B depicts the improved synthesis approach used in these examples.

Previously, CETZOLE 1 was synthesized as the (R)-isomer through the strategy illustrated in Scheme 1 (FIG. 3A) in which Sonogashira coupling, Stille coupling, and Corey-Bakshi-Shibata (CBS) reduction were the key steps. An alternative route to synthesize CETZOLE 1 in high yields was developed, using milder reaction conditions and avoiding the use of toxic tin reagents, as shown in Scheme 2 (FIG. 3B). Stetter condensation of ethyl vinyl ketone 9 with the thiazole aldehyde 8 in the presence of thiazolium salt catalyst 10 in anhydrous THF gave the 1,4-addition product 11. Without purification, intramolecular aldol-type cyclization of 11 in anhydrous ethanol in the presence of sodium hydroxide gave the bromo-ketone 12 in quantitative yields. Sodium borohydride reduction of 12 afforded the alcohol 13. Palladium-catalyzed Sonogashira coupling of bromo-ketone 12 with trimethylsilyl-acetylene afforded 5, which was desilylated using potassium carbonate in methanol to obtain 7. Cytotoxicity studies have shown that the racemic alcohol is equipotent with the R-enantiomer of CETZOLE-1 and, thus, the stereochemistry at the chiral center has no effect on cytotoxicity. Therefore, the ketone 7 was reduced with sodium borohydride in methanol to obtain the final product 1 as a racemic mixture. This approach was used in the synthesis of CETZOLE 1 and analogs.

FIG. 4 depicts Scheme 3, showing the approach to functionalize CETZOLE ketone 7 α-carbon. (i) is the unsuccessful alkylation via enolate generated with LDA. (ii) is the synthesis of oxime 14 with tert-butyl nitrite.

In order to obtain SAR information to optimize antiproliferative potency and for CETZOLE probe design, the synthesis of a library of analogues was undertaken. Position a to the alcohol function of CETZOLE is amenable to chemical modification. Ketone 7 was used as the precursor for functionalization at this position (Scheme 3, FIG. 4). Attempted mono benzylation at the homo-allylic position using benzyl bromide 15 via the enolate generated with LDA led mainly to di-alkylation product with some polymerization. The installation of an oxime at the same position gave oxime 14 in quantitative yields. This was performed by first dissolving the ketone 7 in acidified methanol at 0° C., and then adding tert-butyl nitrite, resulting in the product precipitating out.

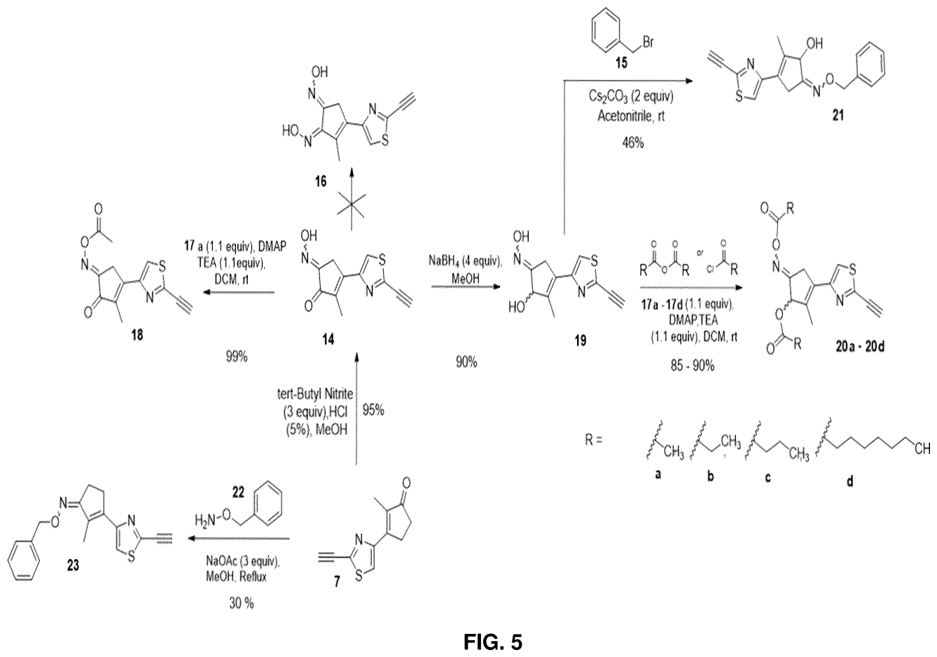

Acetylation of the keto-oxime 14 with acetic anhydride gave the oxime acetate 18 (Scheme 4, FIG. 5). Reduction of the keto-oxime 14 with sodium borohydride in methanol afforded 19, which was acylated with the appropriate acyl chloride or anhydride 17a-17d in the presence of 4-dimethylaminopyridine to obtain the diacyl derivatives 20a-20c, 20e. Attempts to prepare the di-oxime intermediate 16 by reacting 14 with hydroxylamine hydrochloride were not successful. To study the effects of structural modification of CETZOLE with bulky groups, a second set of analogs (21 and 23) was synthesized. These compounds were to be used as templates for making probes for target protein identification. The oxime alcohol 19 was alkylated by dropwise addition of benzyl bromide 15 in the presence of cesium carbonate in acetonitrile to obtain the oxime benzyl ether 21. Heating the ketone 7 under reflux with a mixture of O-benzylhydroxylamine 22 and sodium acetate gave the O-benzyl oxime analog 23. (Scheme 4, FIG. 5).

### Cytotoxic Activity of CETZOLE Analogs

Having synthesized the CETZOLE analogs, their cytotoxic activity was evaluated. The mesenchymal human non-small cell lung tumor cell line NCI-H522 is very sensitive to the ferroptotic agent CETZOLE 1. In these examples, the compounds were screened against the NCI-H522 cell line, as well as the Ras-mutant fibrosarcoma cell line HT-1080, late-stage breast cancer cell line MDA-MB 231, the cancer cell clone NCI-H522 GFP-SLC7A11 #8 in which GFP-tagged X_{c}⁻ antiporter protein SLC7A11 has been overexpressed by viral transfection, and the GFP-tagged retroviral clone NCI-H522 RV-GFP, for antiproliferative activity. As the GFP-tagged X_{c}⁻ antiporter protein SLC7A11 is overexpressed in the NCI-H522 GFP-SCL7A11 #8 clone by viral transfection, this cell line is generally less sensitive to ferroptosis than NCI-H522 cells. NCI-H522 RV-GFP is the corresponding control with RV-GFP tag, but without SLC7A11 overexpression. The NARF2 cells derived from human osteosarcoma U2OS cell line are susceptible to ferroptosis; however, higher concentrations of CETZOLE 1 are required to observe this form of cell death as compared to highly sensitive cell lines NCI-H522 and MDA MB 231. The cytotoxicity screening was conducted as described previously. The ferroptotic agent RSL3 had IC₅₀ values between 0.14-2.84 μM against the tested cell lines. The keto oxime 14 and its corresponding acetate 18 were the least active of all the analogues and had IC₅₀ values>20 μM. This is consistent with previous findings that the ketone analogue of CETZOLE 1 is less reactive than CETZOLE 1. The corresponding alcohol 19 had modest IC₅₀ values against NCI-H522, NCI-H522 RV-GFP, MDA-MB 231, and HT-1080 cell lines, but it was not cytotoxic (IC₅₀>20 μM) to the more resistant cell lines NCI-H522 GFP-SLC7A11 and NARF2. Interestingly, the most active analogues were the diacetate 20a, the dipropionate 20b, and the di-butanoate 20c of the oxime alcohol 19, with IC₅₀ values ranging from high nanomolar to low micromolar. The higher activity of these esters may be attributed to them acting as prodrugs of higher lipophilicity and cell membrane permeability. They can undergo hydrolysis within the cell to release the active drug 19. Overexpression of GFP-SLC7A11 reduced sensitivity to most of the compounds tested. The corresponding octanoate 20e had IC₅₀ values>20 μM against all the cell lines, demonstrating that esters with larger alkyl chains are not well tolerated. Whether this is due to an increase in lipophilicity, steric bulk, or any other factor is not known.

TABLE 1
Half maximal inhibitory concentration (IC₅₀) of CETZOLE and CETZOLE analogs

| | | Cancer Cell Lines (μM) | | | | | |
|---|---|---|---|---|---|---|---|
| | | | NCI- | | | | |
| | | | H522 | | | | |
| | | | GFP- | NCI- | | | MDA- |
| | | NCI- | SCL7A | H522 | HT- | | MB- |
| Analogs | Structure | H522 | 11 #8 | Rv-GFP | 1080 | NARF2 | 231 |
| RSL3 | | 1.1 ± 0.89 | 2.84 ± 0.9 | 0.31 ± 0.04 | 0.14 ± 0.08 | 2.46 ± 0.76 | 0.22 ± 0.02 |
| CET- | | 2.56 ± 0.93 | 10.31 ± 3.6 | 2.71 ± 0.41 | 3.07 ± 1.3 | 14.9 ± 1.3 | 6.28 ± 0.73 |
| ZOLE 1 | | | | | | | |
| 14 | 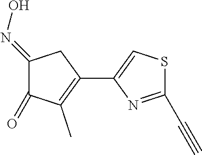 [SMILES: C#Cc1nc(C2=C(C)C(=O)/C(=N/O)C2)cs1] | >20 | >20 | >20 | 5.29 ± 2.6 | >20 | 8.05 ± 0.63 |
| 19 | 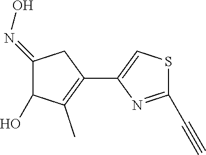 [SMILES: C#Cc1nc(C2=C(C)C(O)/C(=N/O)C2)cs1] | 4.68 ± 1.03 | >20 | 5.55 ± 0.01 | 5.77 ± 0.67 | >20 | 6.57 ± 0.50 |
| 18 | 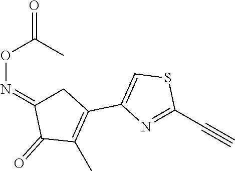 [SMILES: C#Cc1nc(C2=C(C)C(=O)/C(=N/OC(C)=O)C2)cs1] | 10.97 ± 1.53 | >20 | >20 | >20 | >20 | 18.54 ± 1.7 |
| 20a | 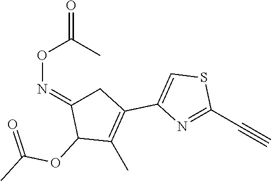 [SMILES: C#Cc1nc(C2=C(C)C(OC(C)=O)/C(=N/OC(C)=O)C2)cs1] | 0.17 ± 0.08 | 1.59 ± 0.49 | 0.26 ± 0.11 | 0.14 ± 0.06 | 7.26 ± 2.01 | 0.23 ± 0.02 |
| 20b | 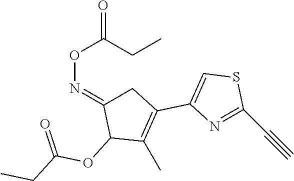 [SMILES: C#Cc1nc(C2=C(C)C(OC(=O)CC)/C(=N/OC(=O)CC)C2)cs1] | 0.12 ± 0.06 | 2.06 ± 0.54 | 0.59 ± 0.22 | 0.15 ± 0.02 | 13.38 ± 3.3 | 0.18 ± 0.03 |
| 20e | 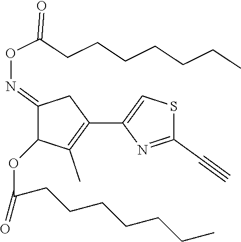 [SMILES: C#Cc1nc(C2=C(C)C(OC(=O)CCCCCCC)/C(=N/OC(=O)CCCCCCC)C2)cs1] | >20 | >20 | >20 | >20 | >20 | 19.1 ± 2.8 |
| 20c | 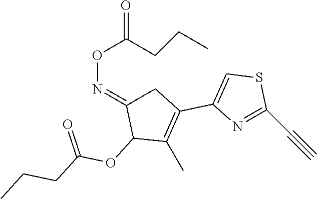 [SMILES: C#Cc1nc(C2=C(C)C(OC(=O)CCC)/C(=N/OC(=O)CCC)C2)cs1] | 0.13 ± 0.09 | 1.93 ± 0.8 | 0.28 ± 0.13 | 0.19 ± 0.09 | 5.95 ± 1.80 | 0.83 ± 0.15 |
| 21 | 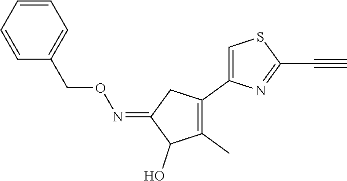 [SMILES: C#Cc1nc(C2=C(C)C(O)/C(=N/OCc3ccccc3)C2)cs1] | 1.18 ± 0.8 | 2.17 ± 1.5 | 3.02 ± 1.7 | 0.93 ± 0.12 | >20 | 13.4 ± 5.3 |
| 23 | 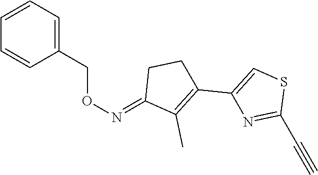 [SMILES: C#Cc1nc(C2=C(C)/C(=N/OCc3ccccc3)CC2)cs1] | 1.36 ± 0.11 | 2.41 ± 1.16 | 2.54 ± 0.44 | 0.81 ± 0.14 | 19.28 ± 5.9 | 4.17 ± 0.39 |

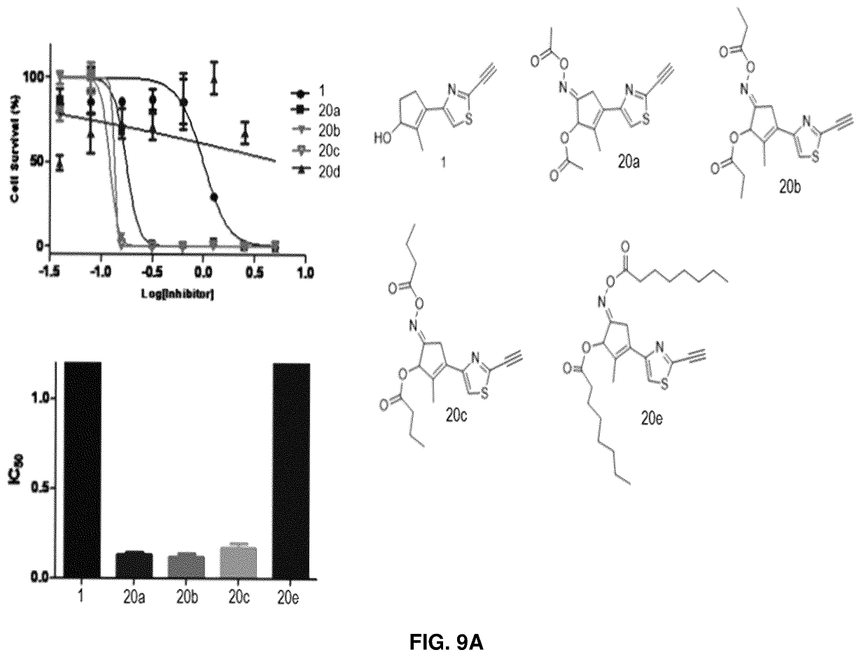

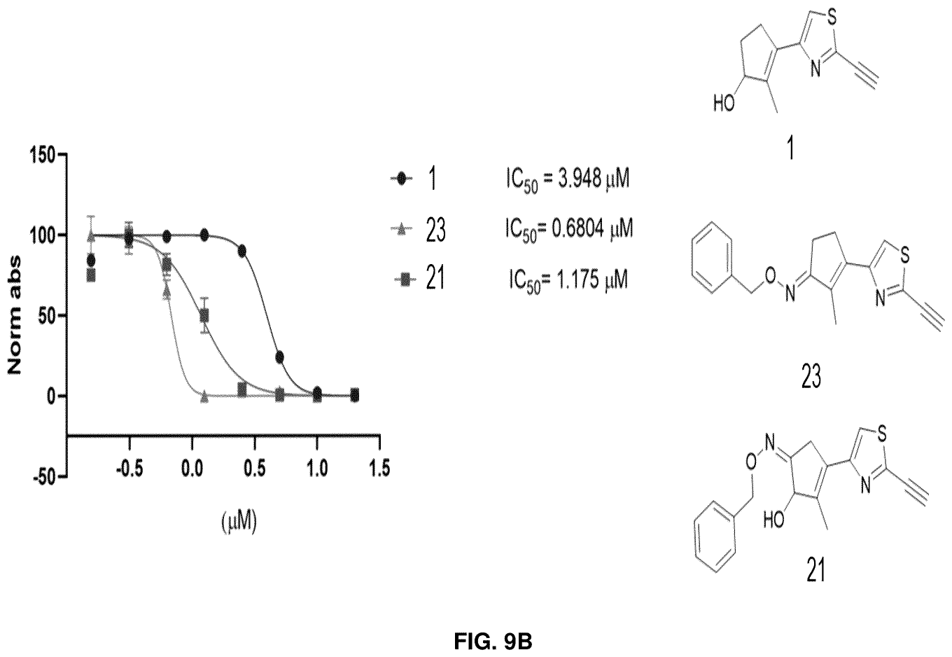

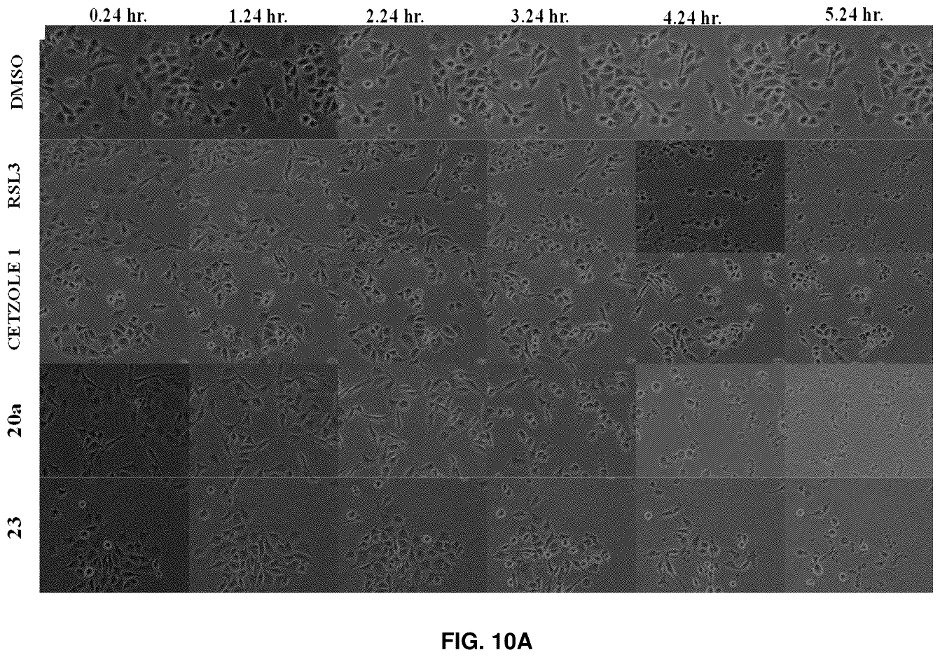

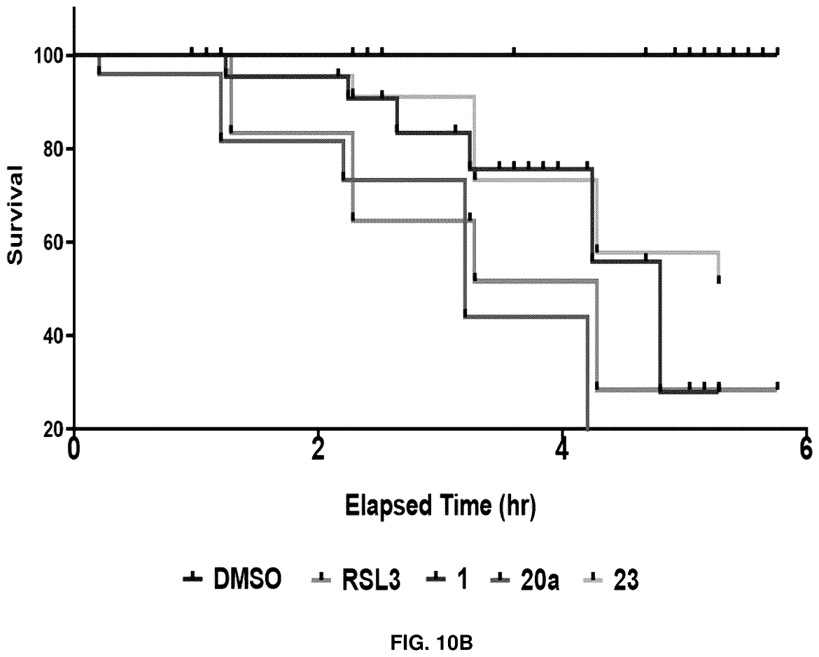

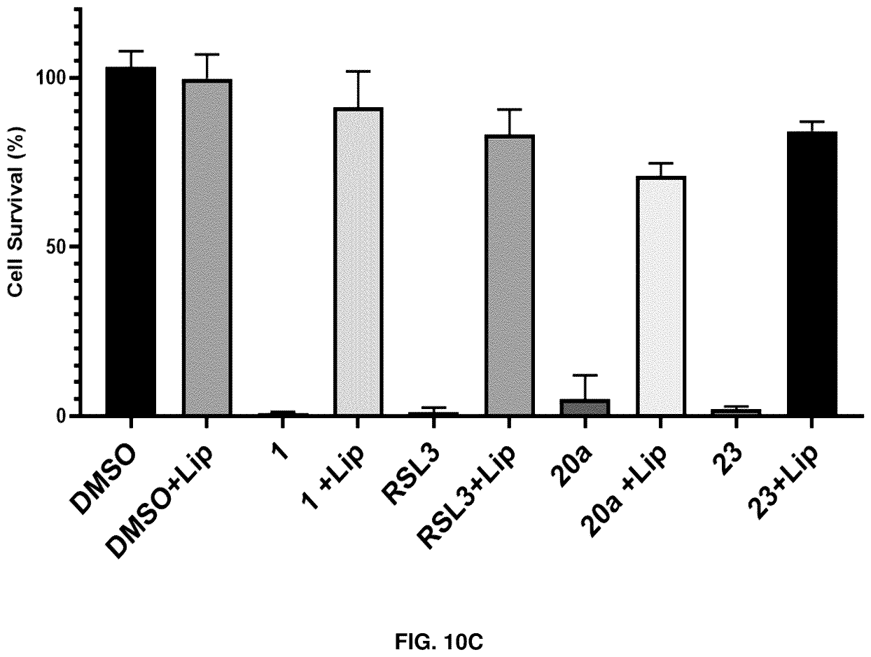

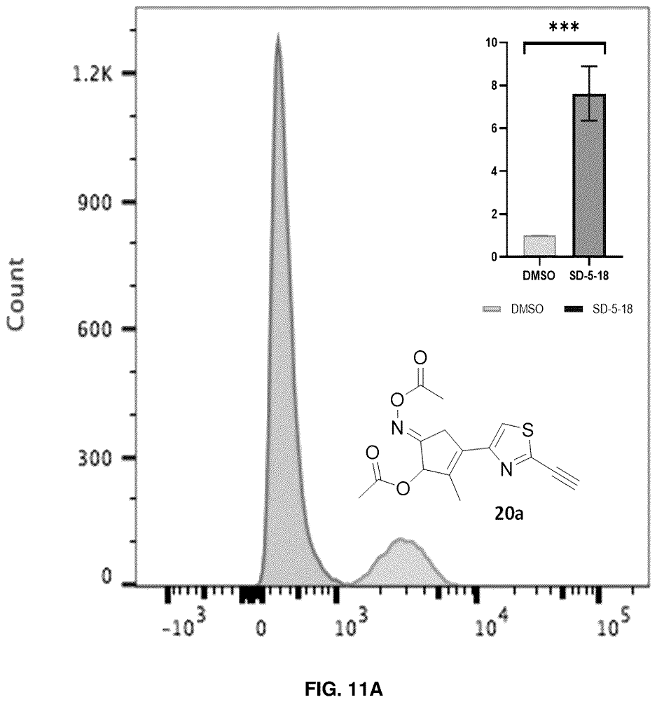

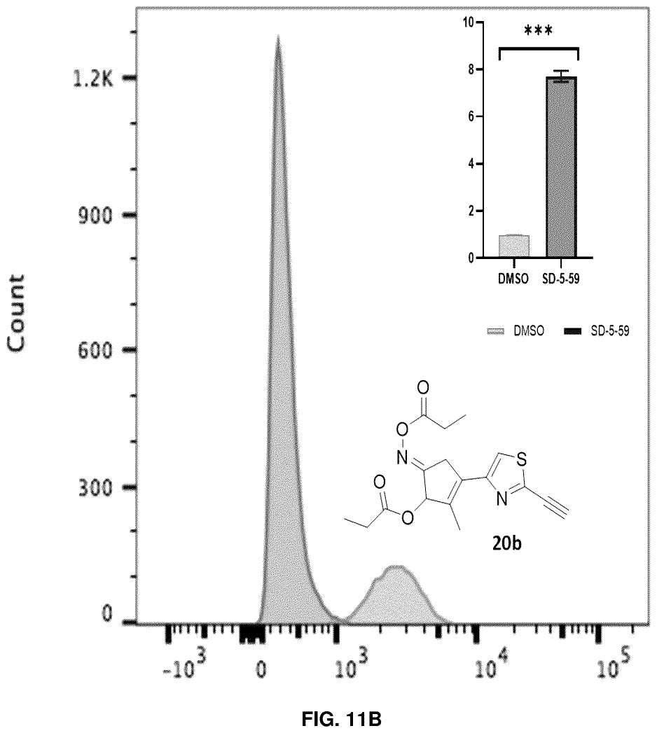

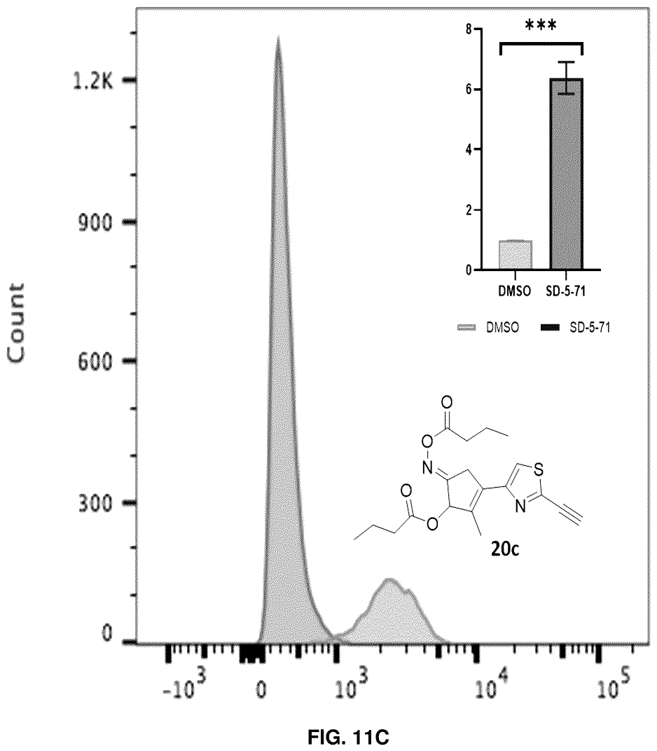

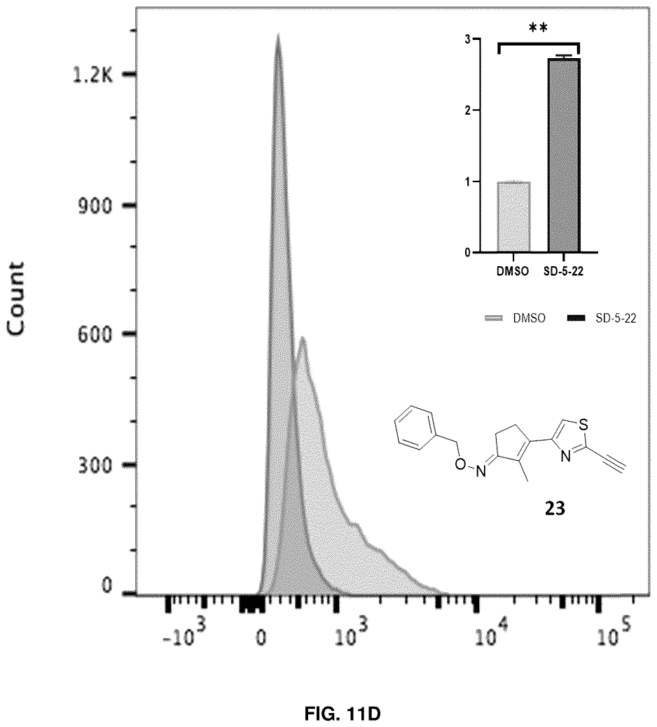

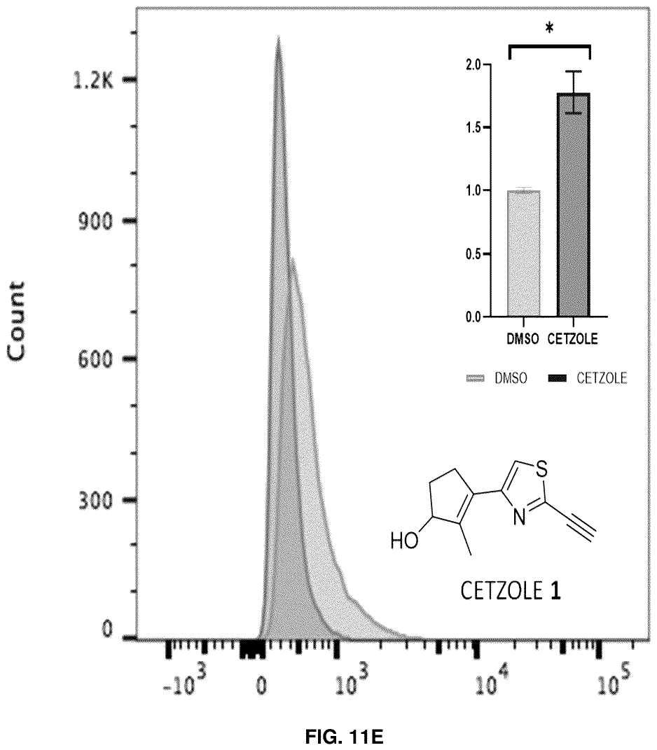

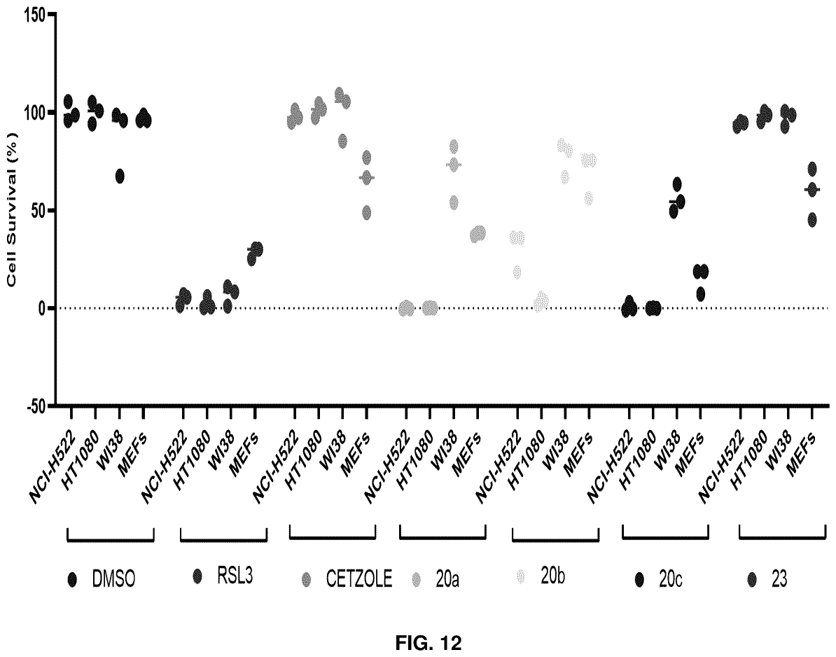

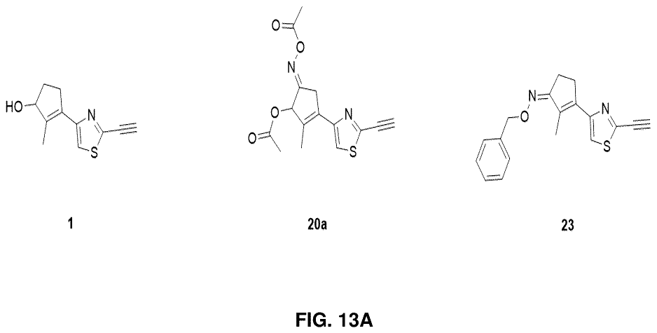

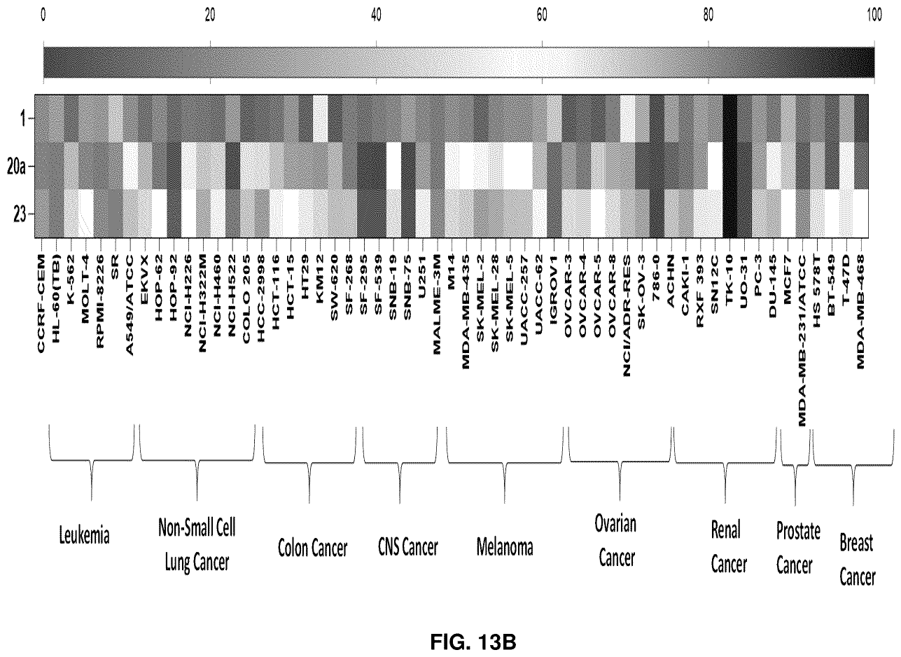

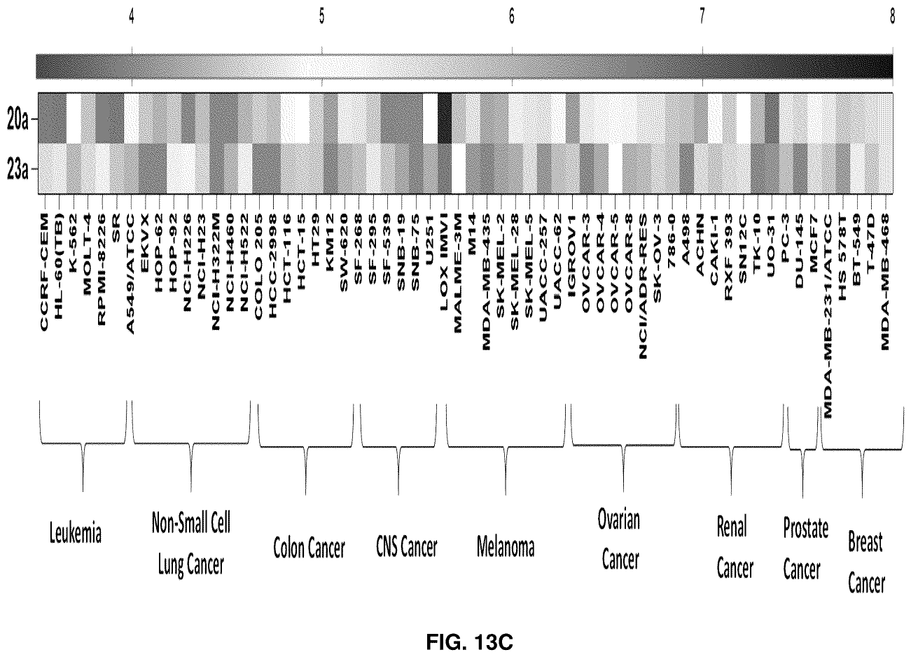

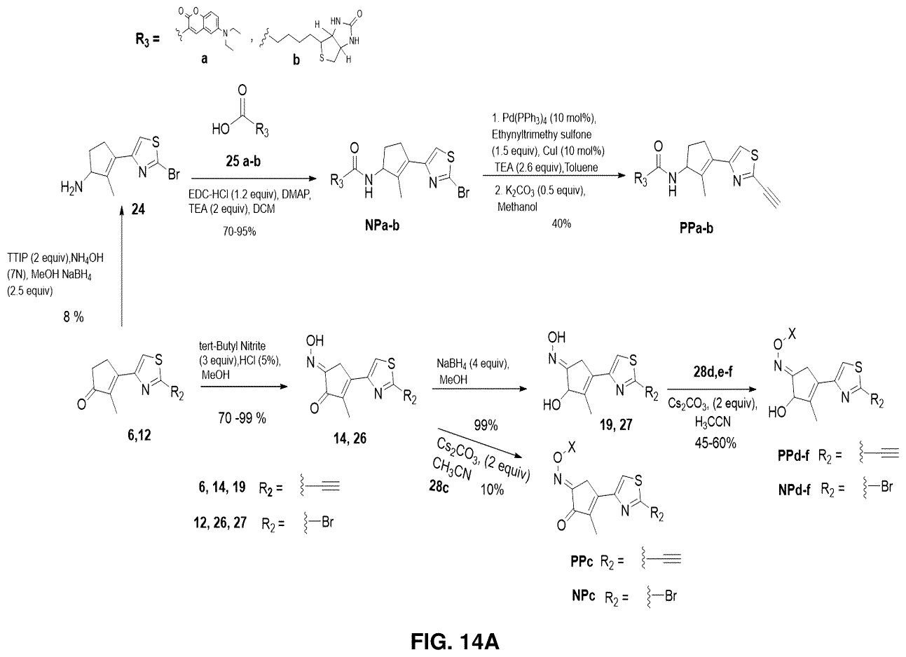

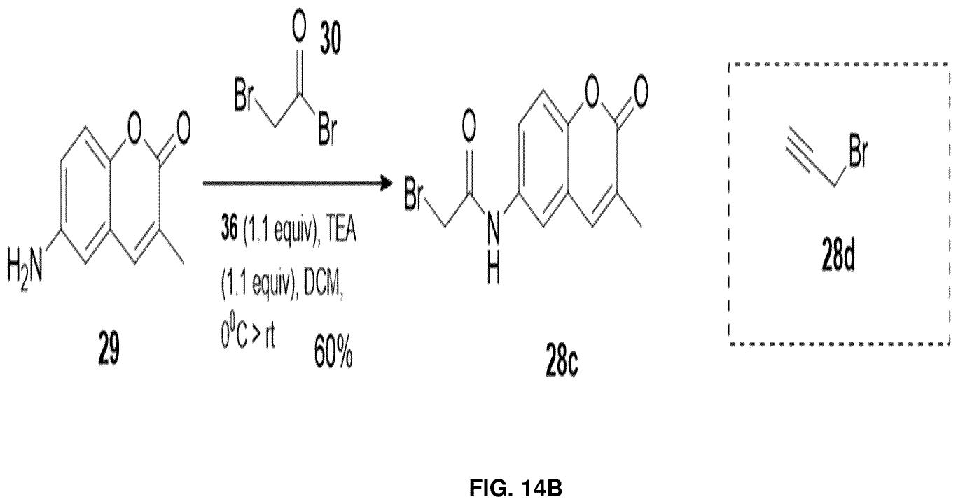

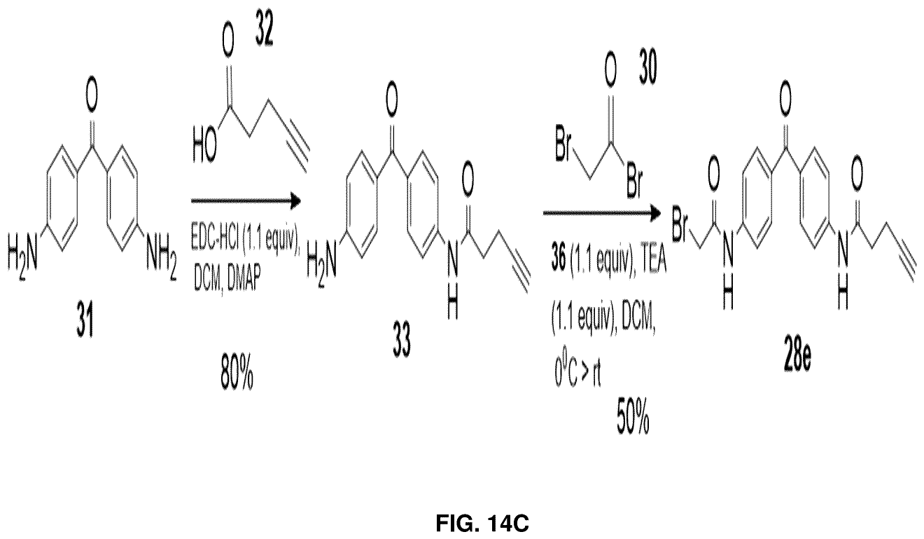

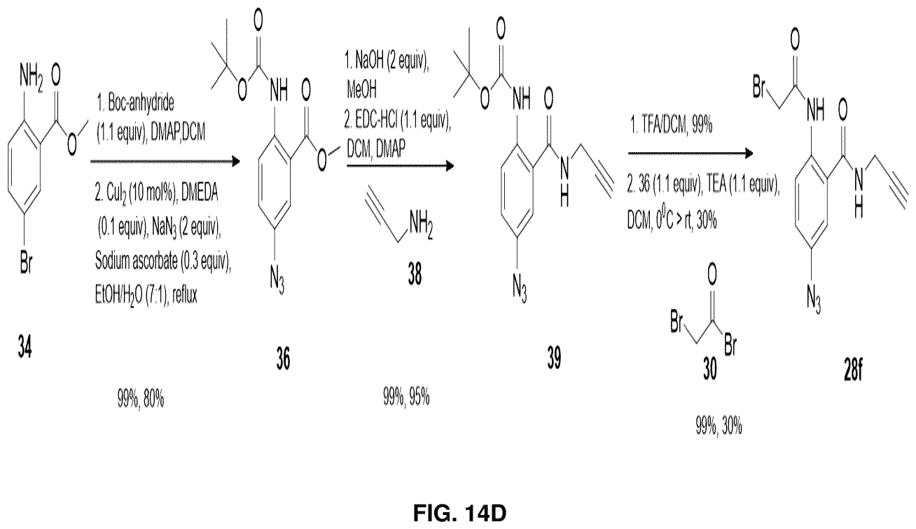

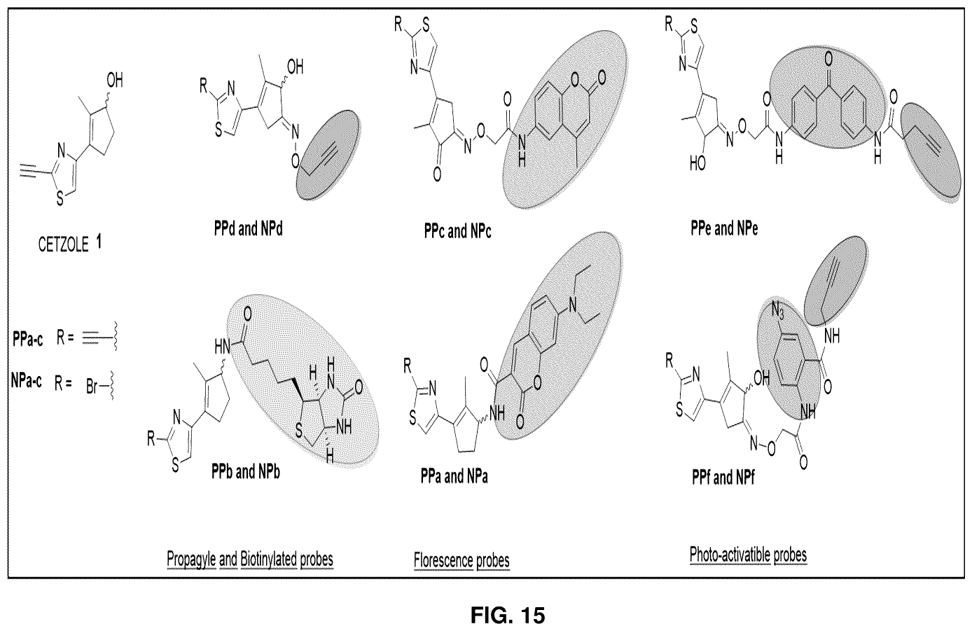

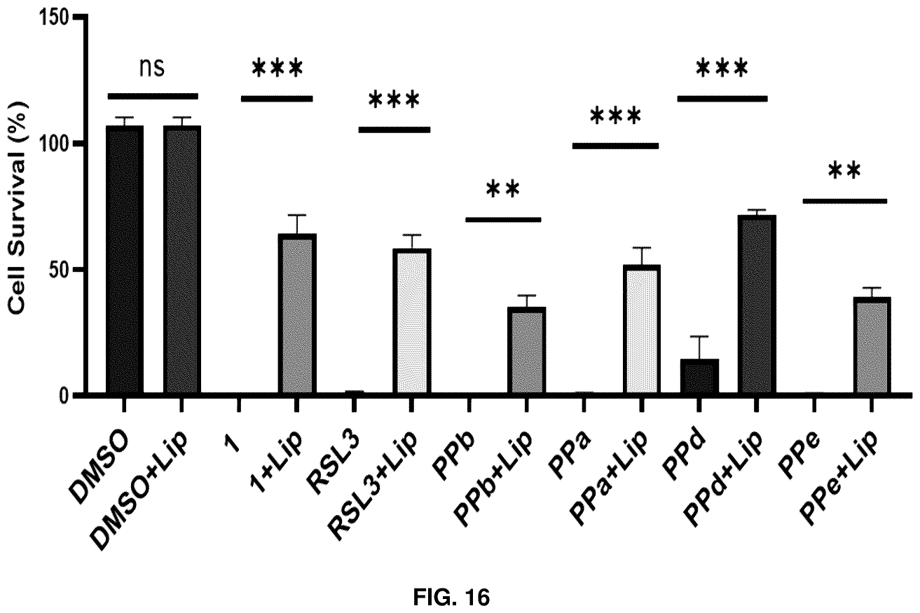

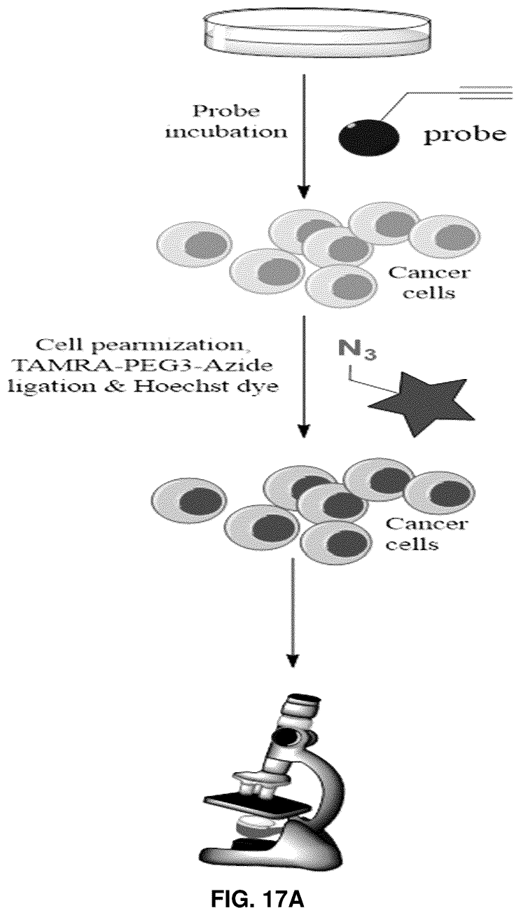

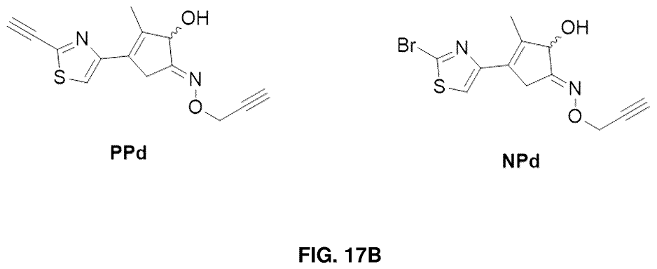

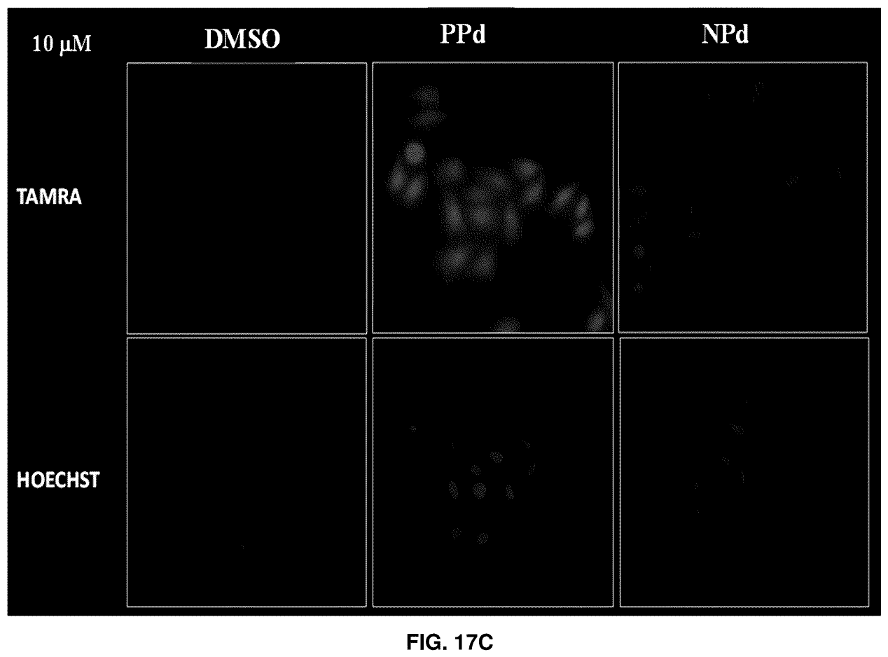

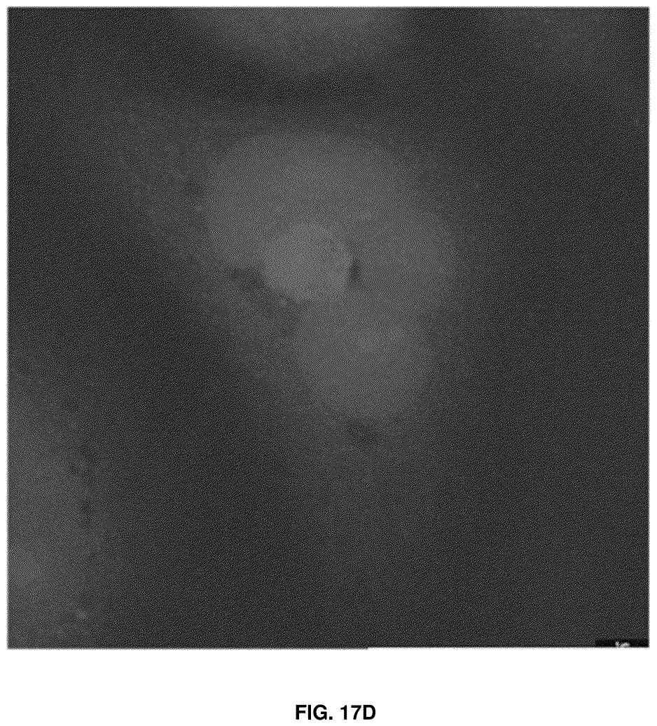

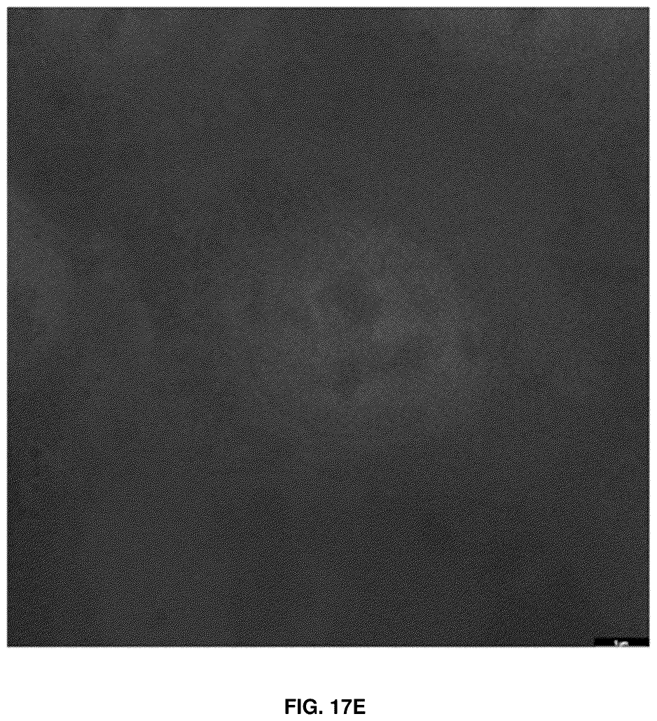

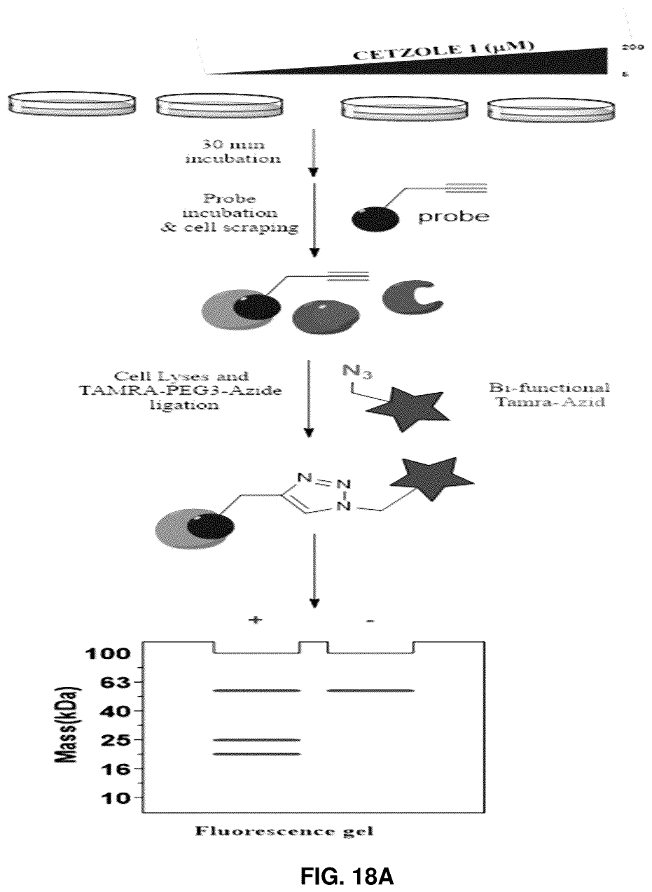

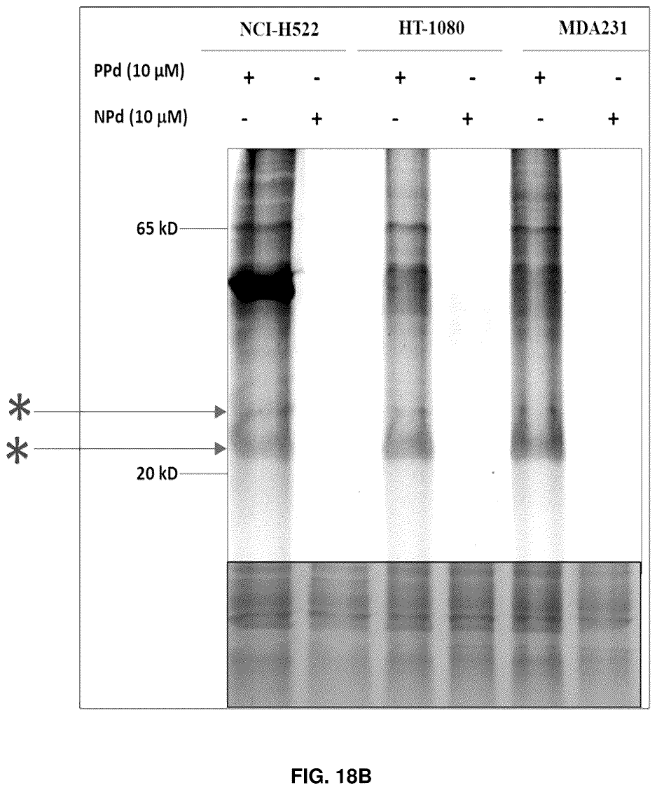

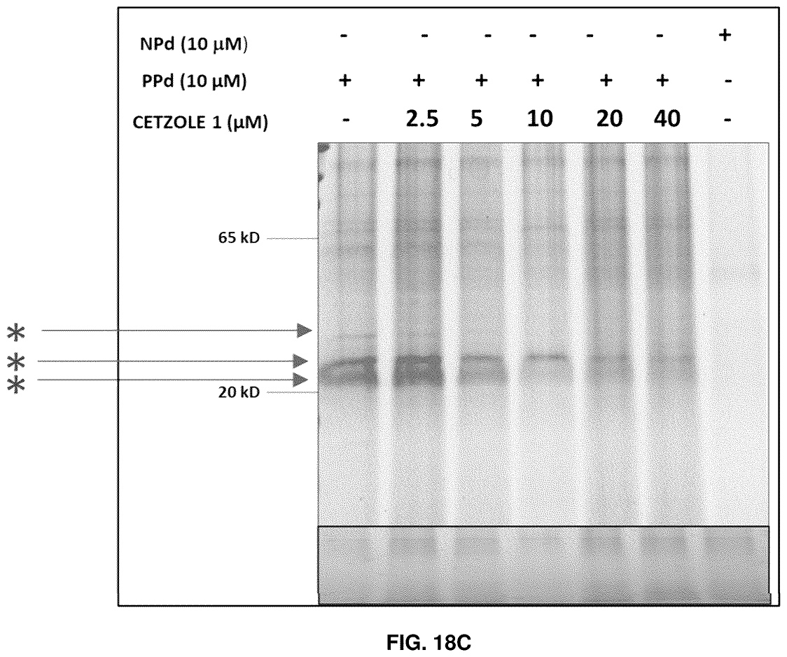

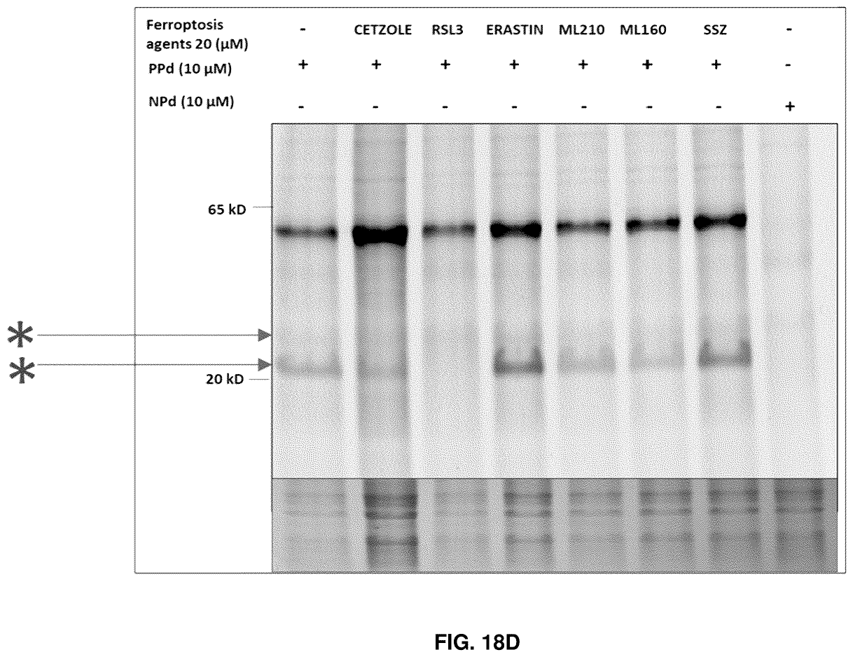

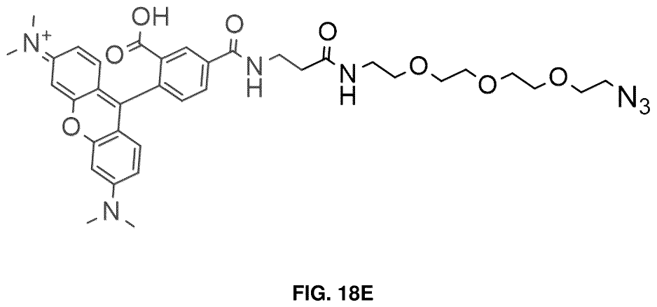

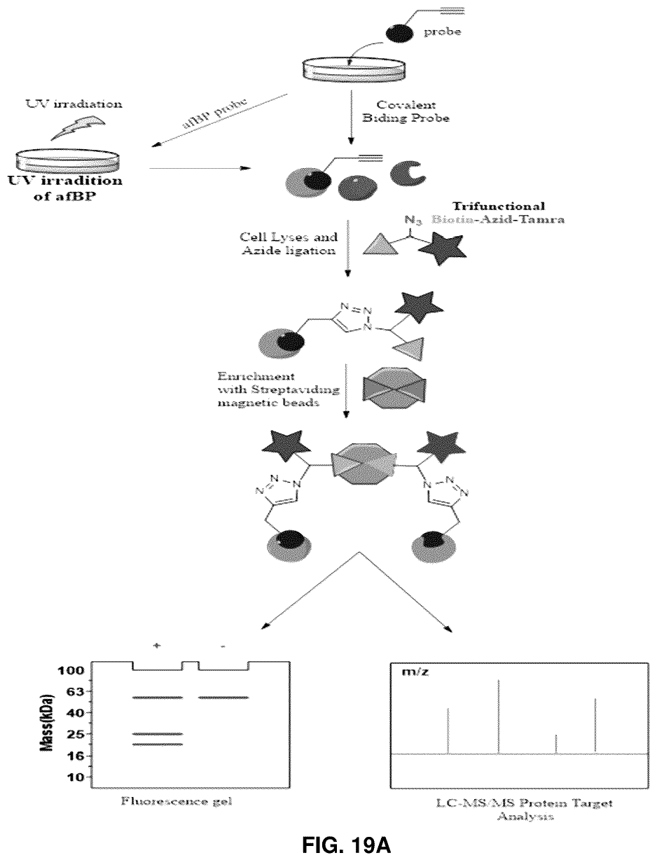

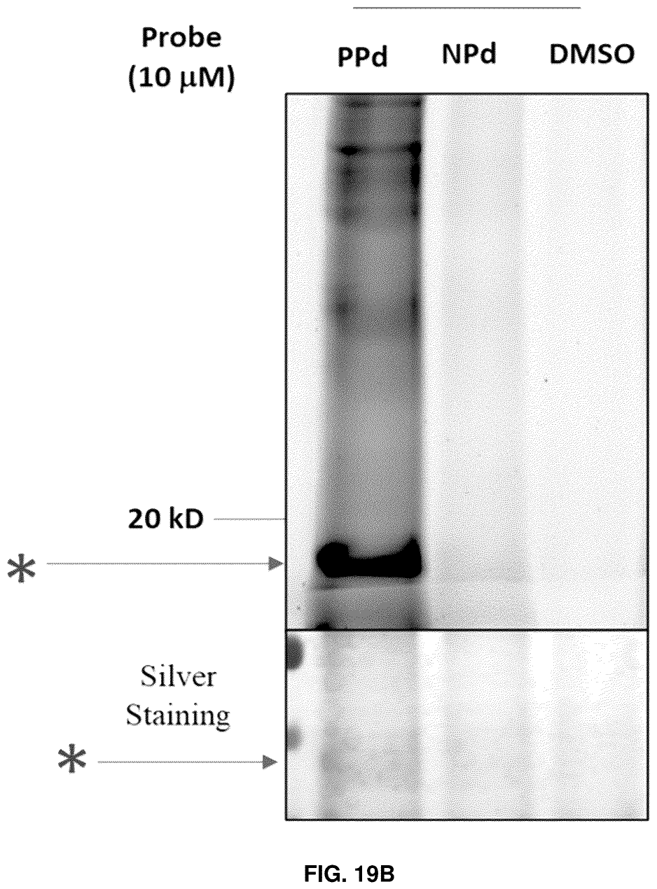

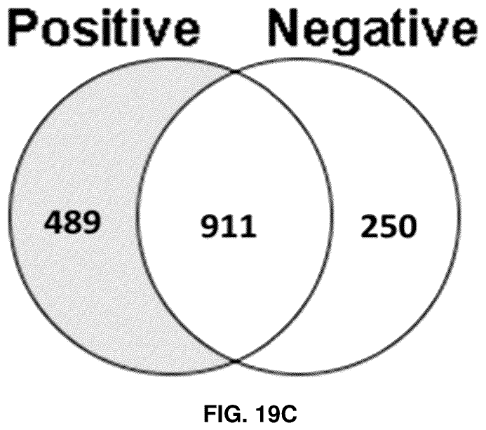

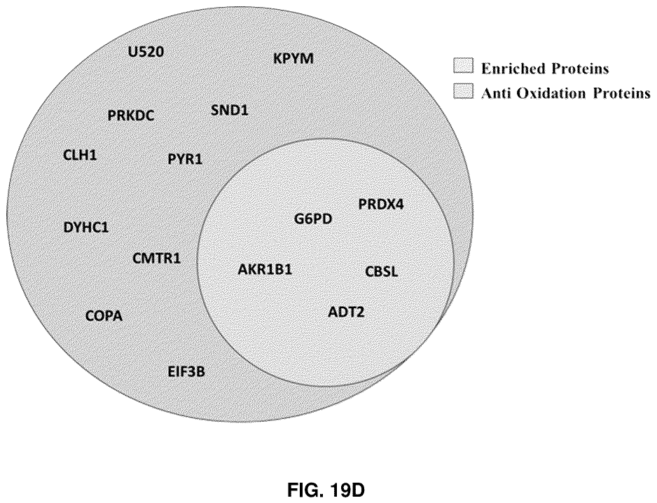

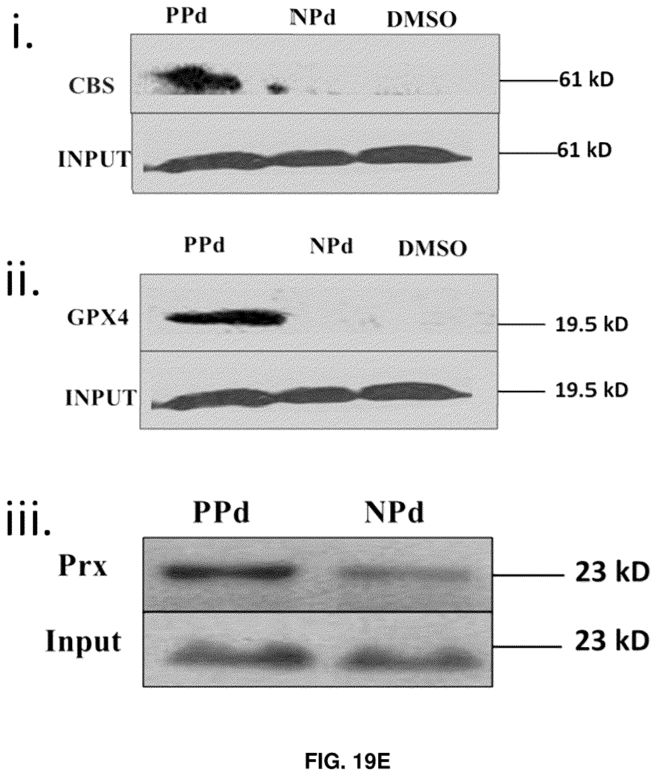

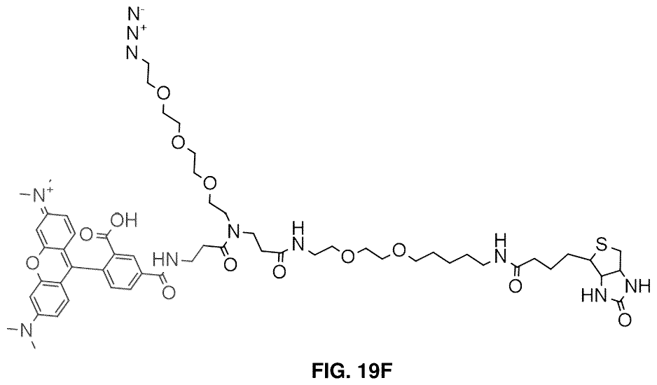

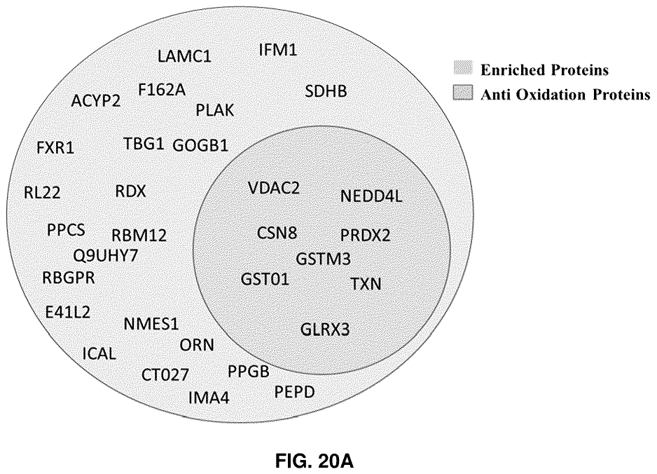

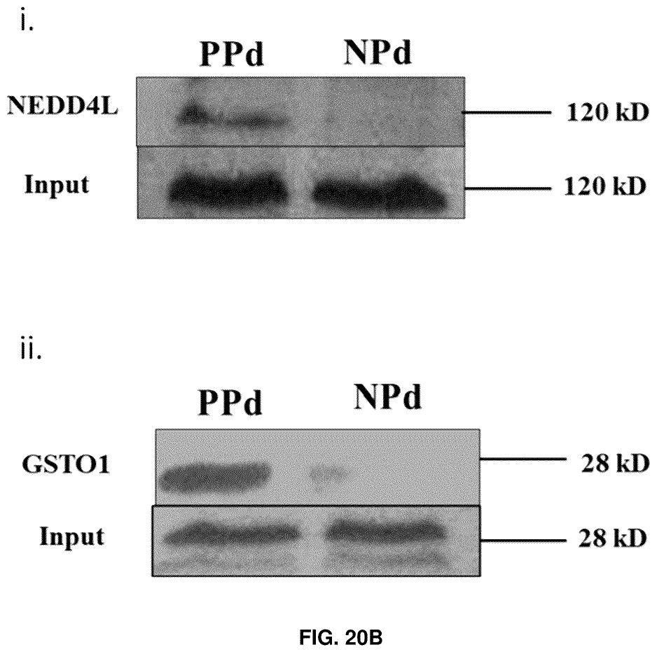

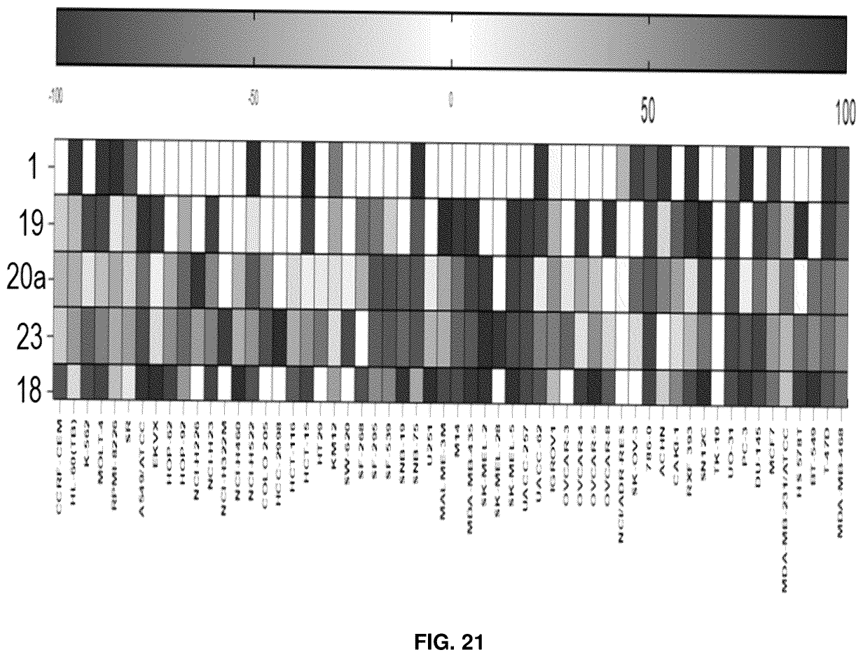

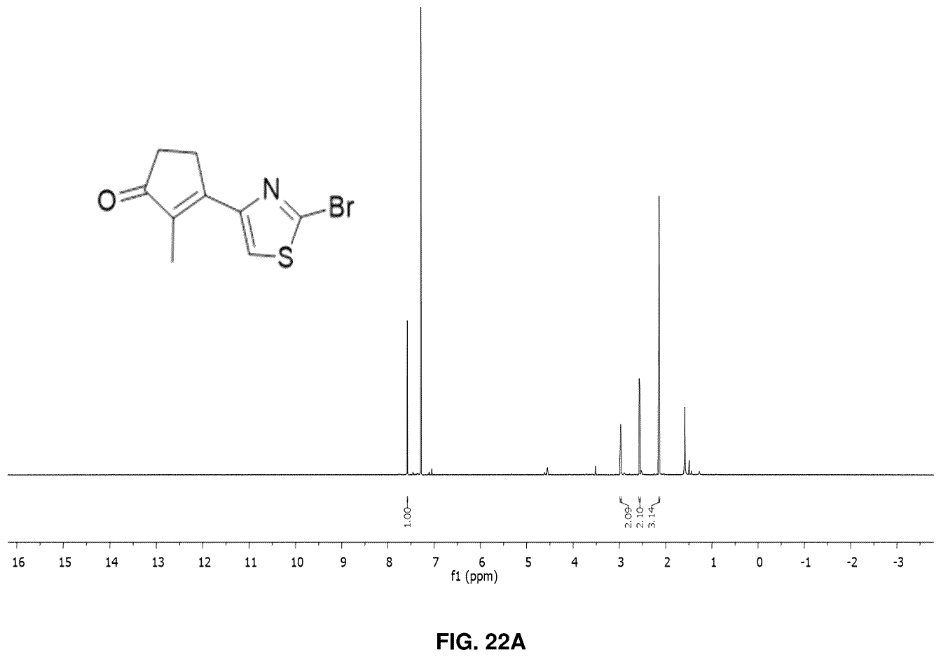

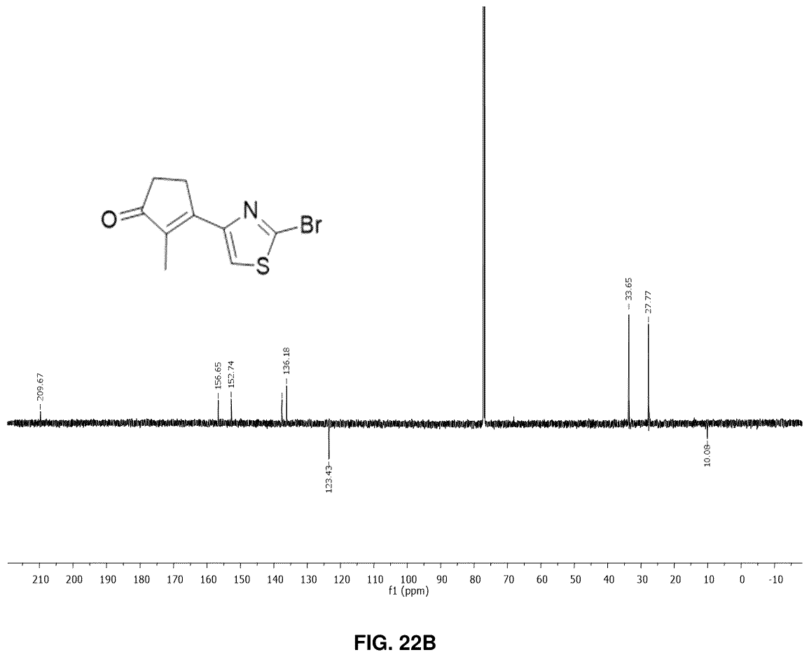

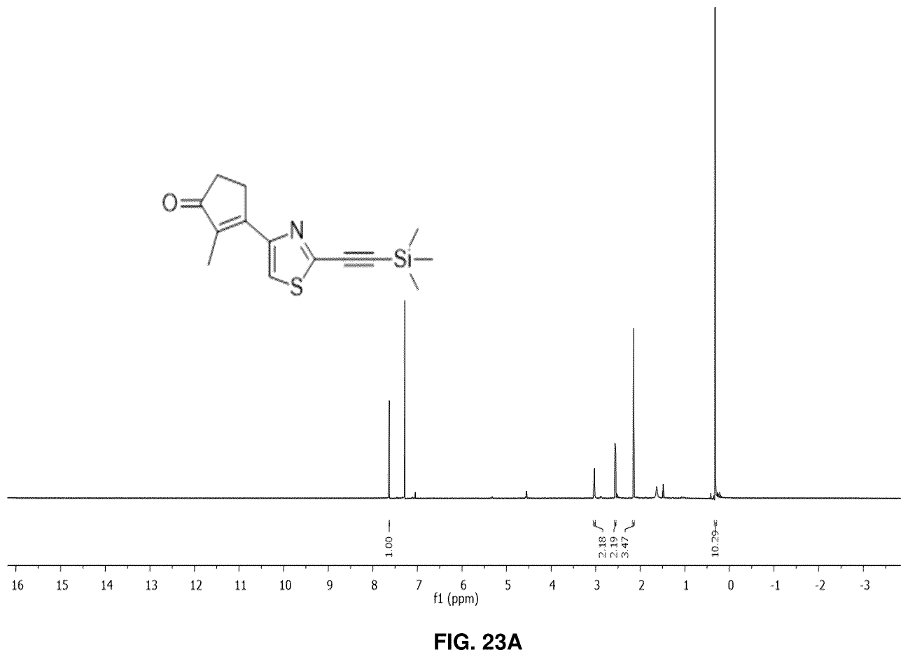

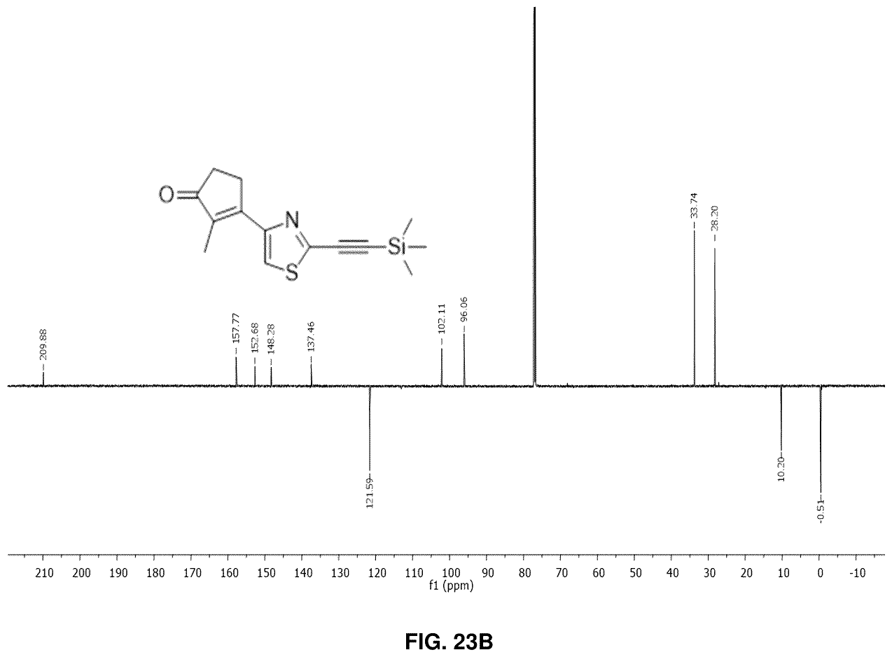

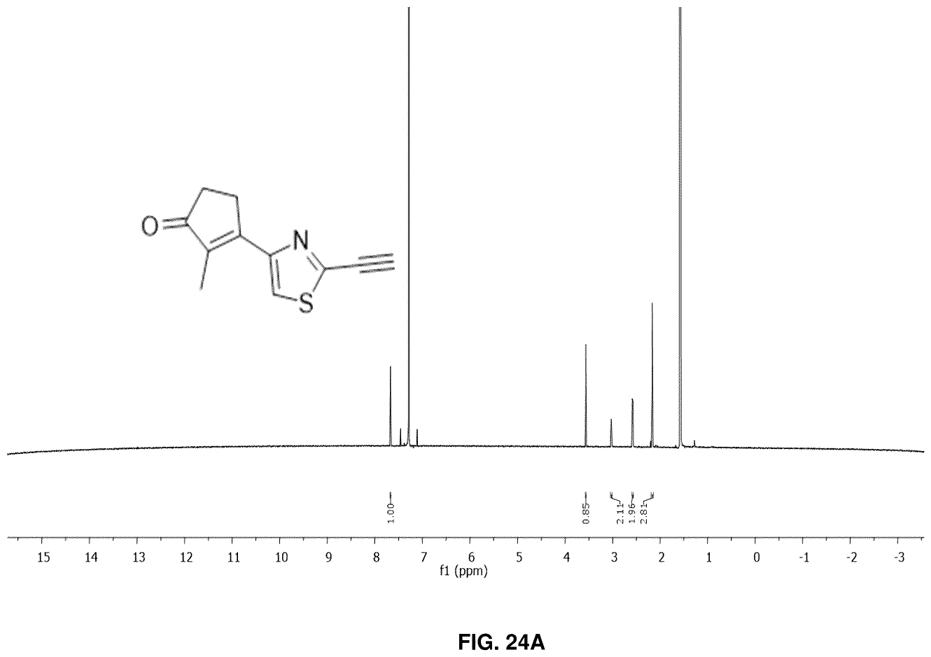

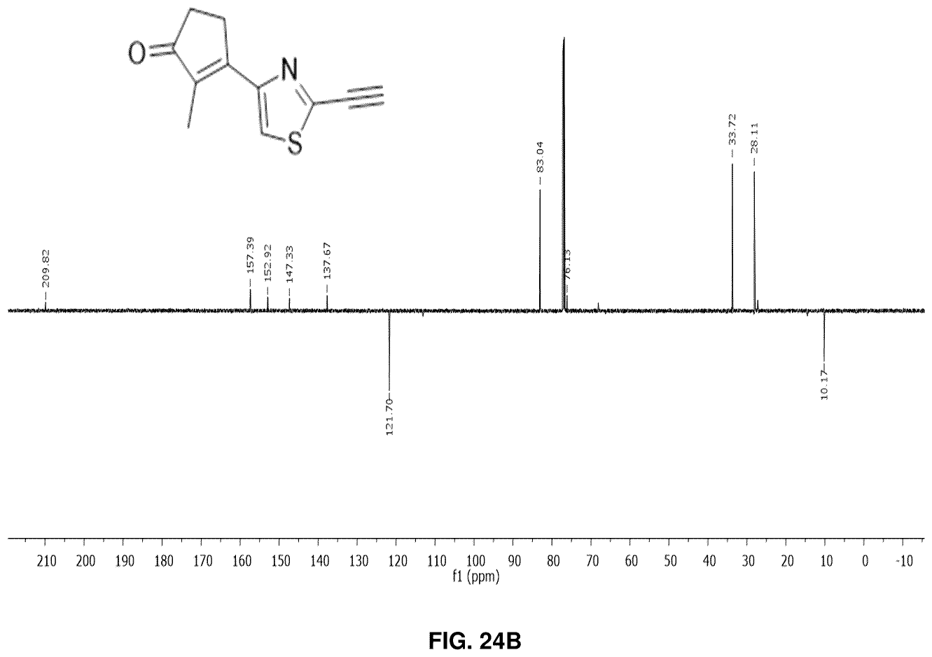

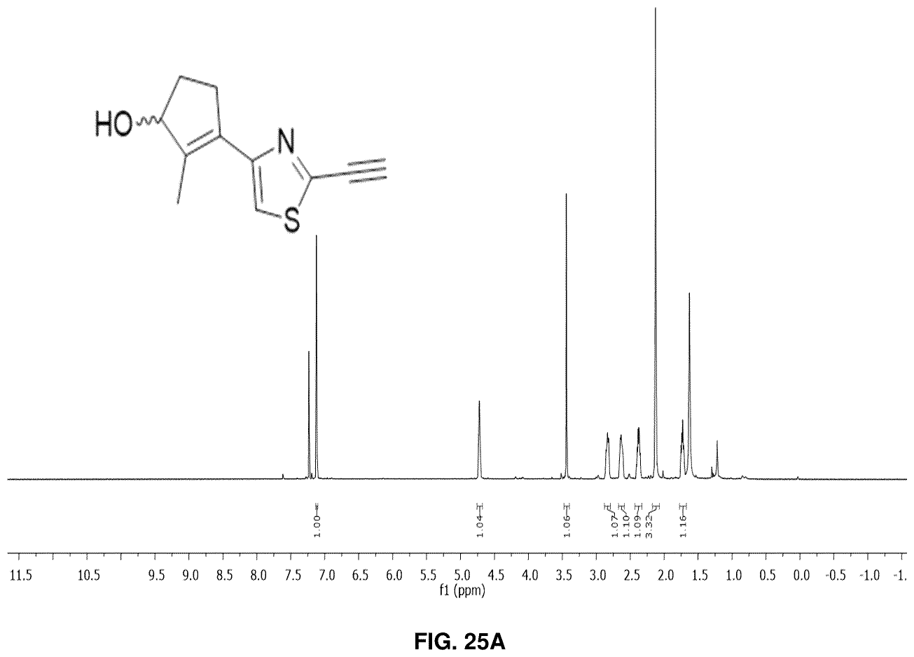

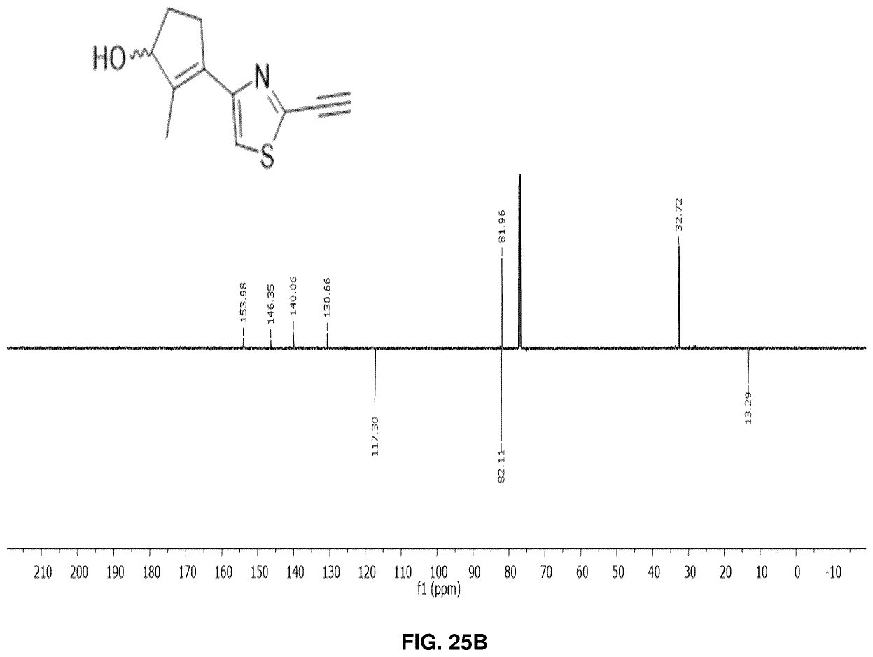

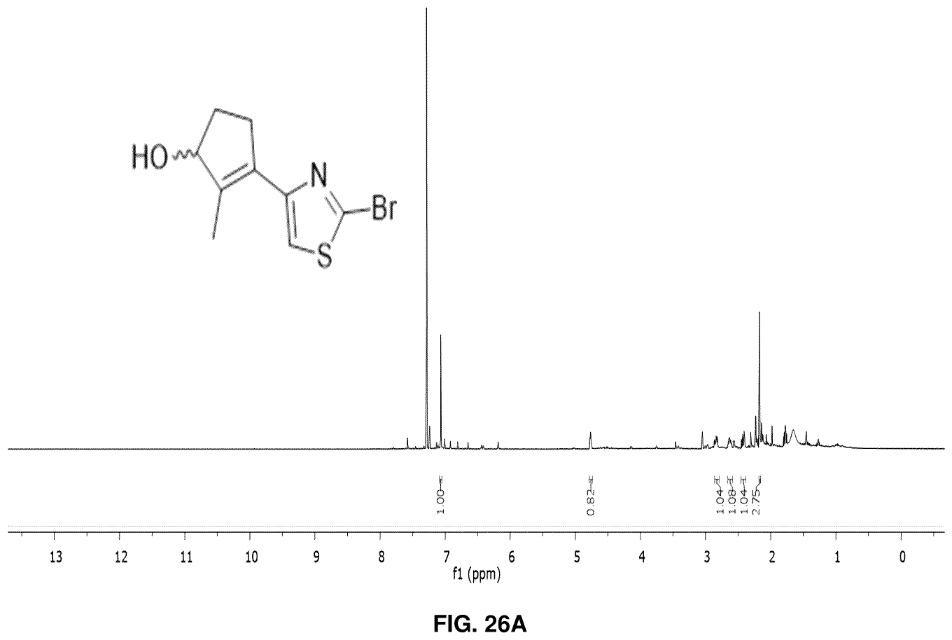

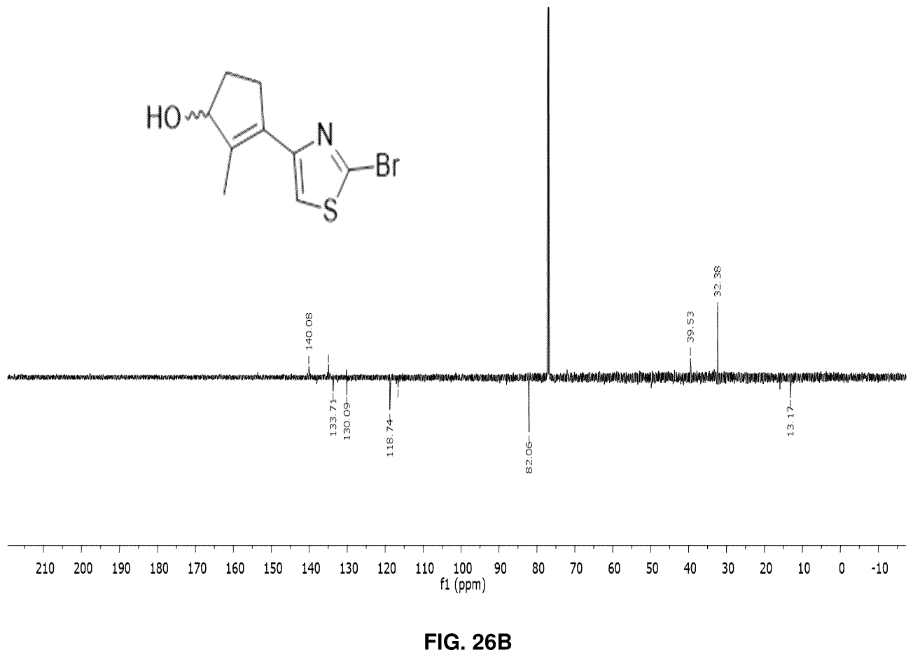

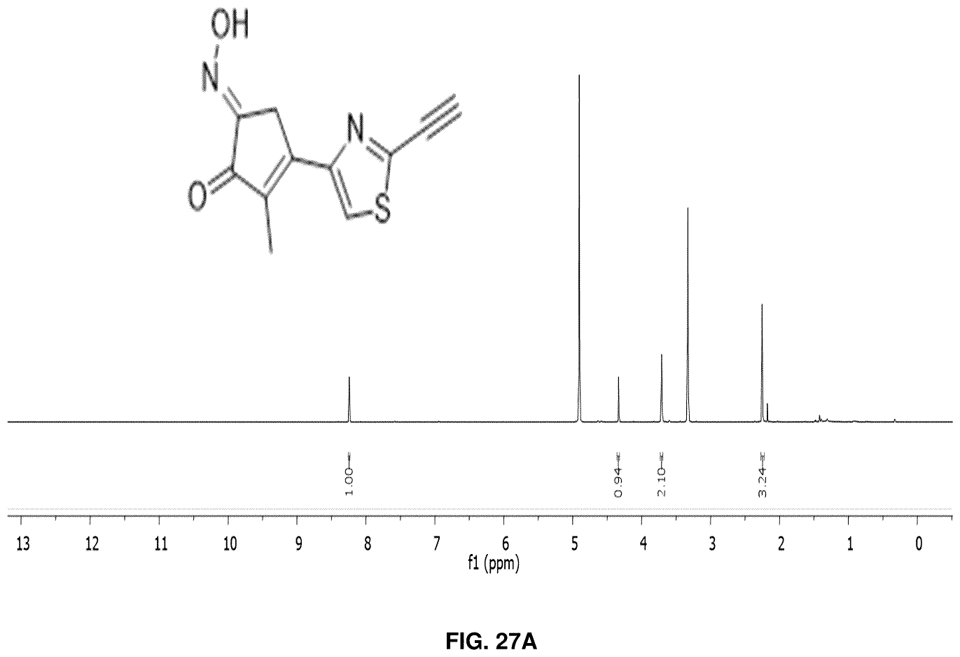

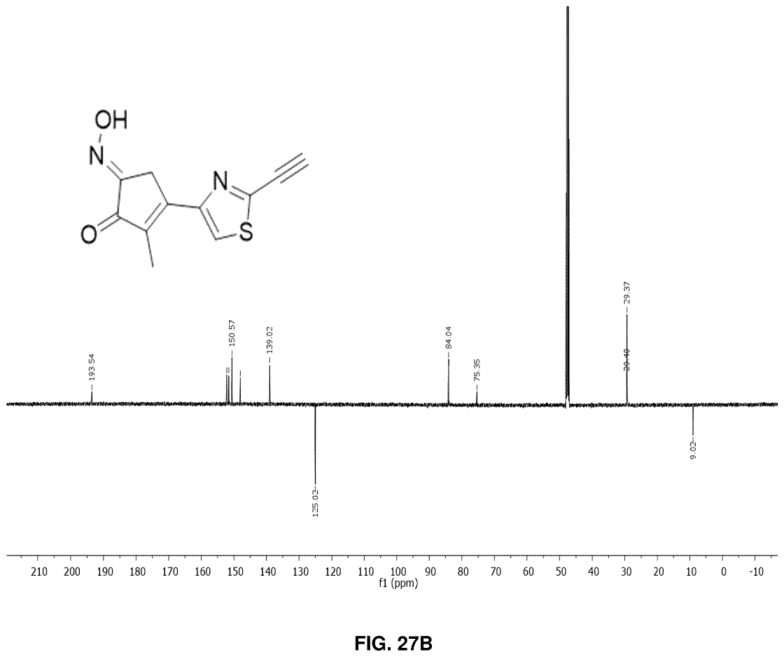

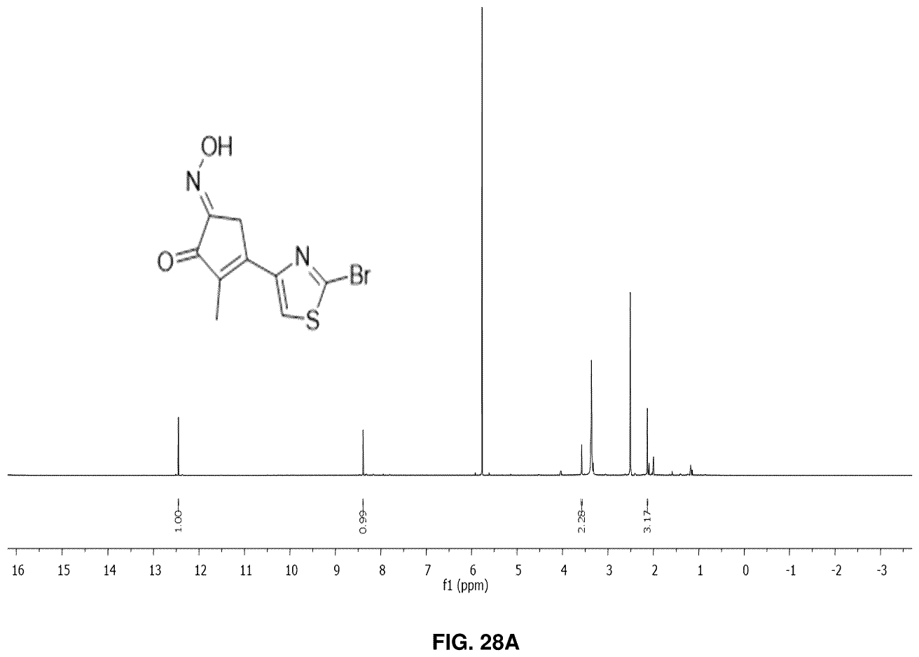

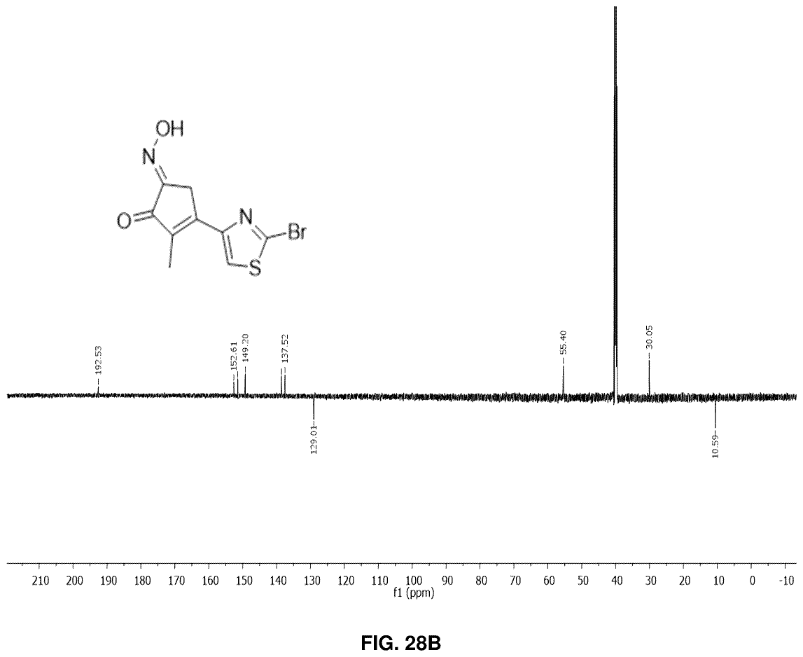

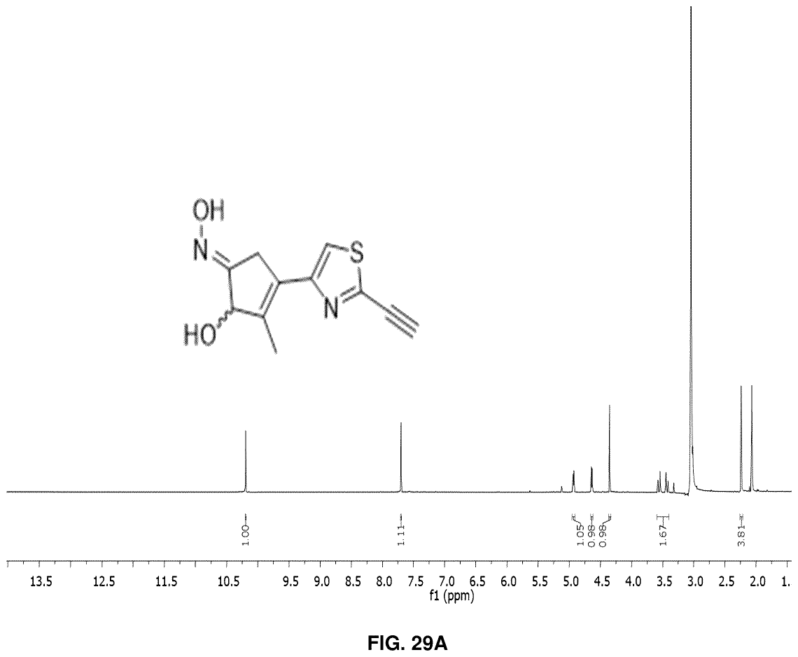

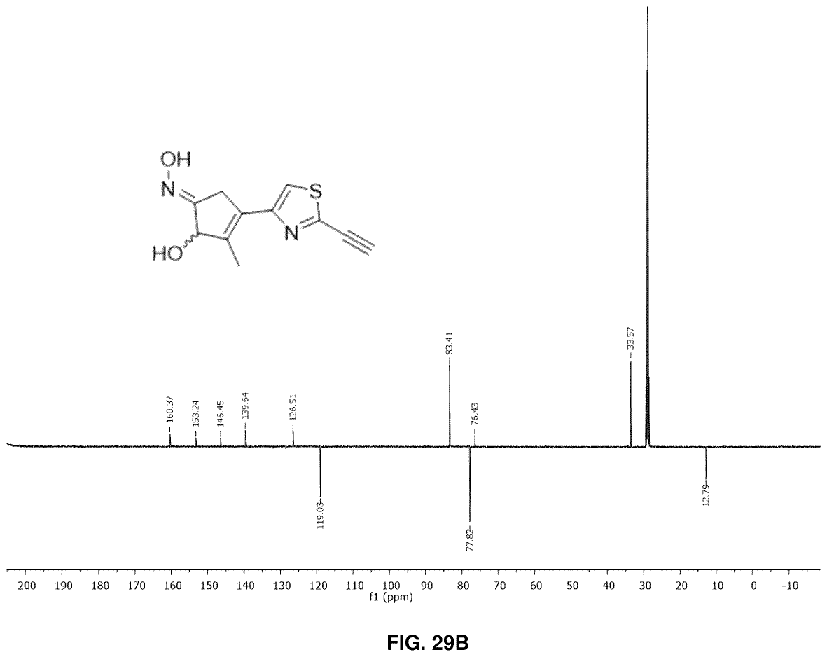

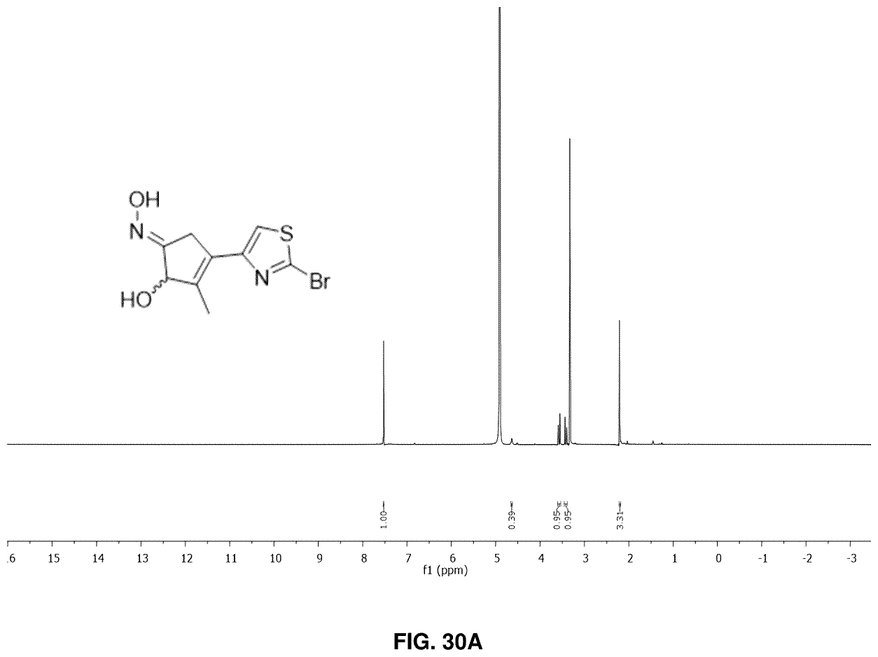

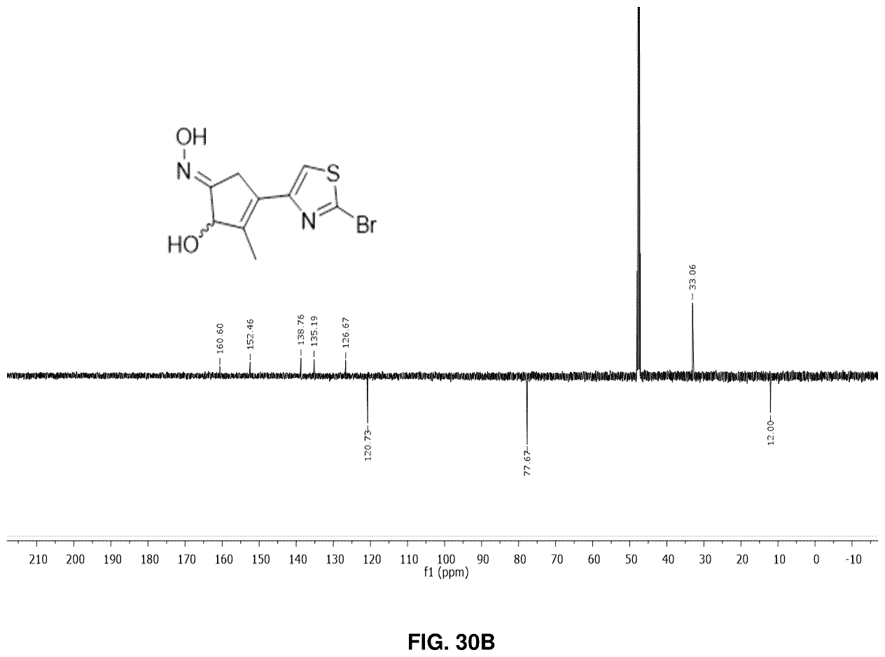

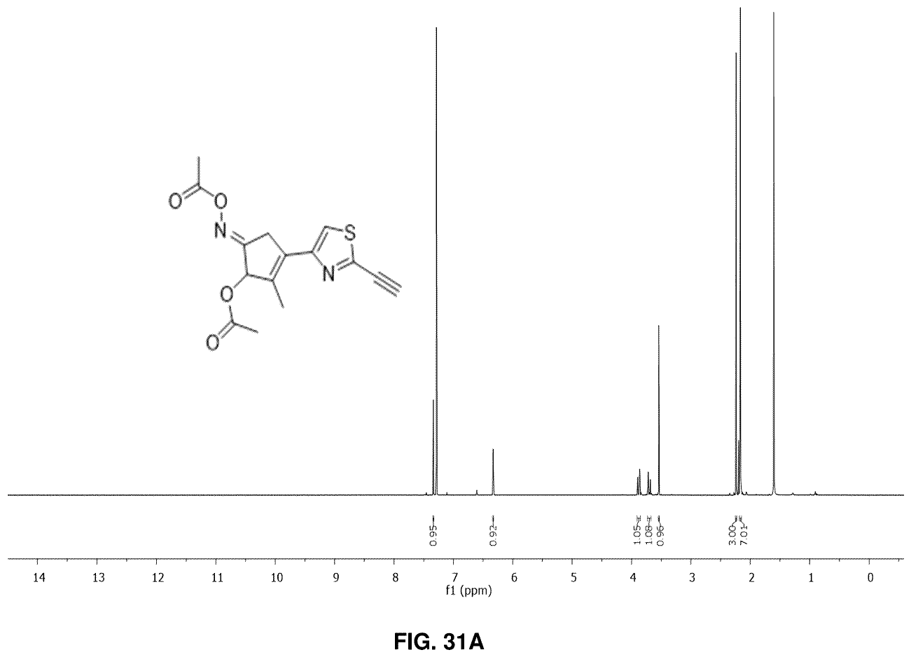

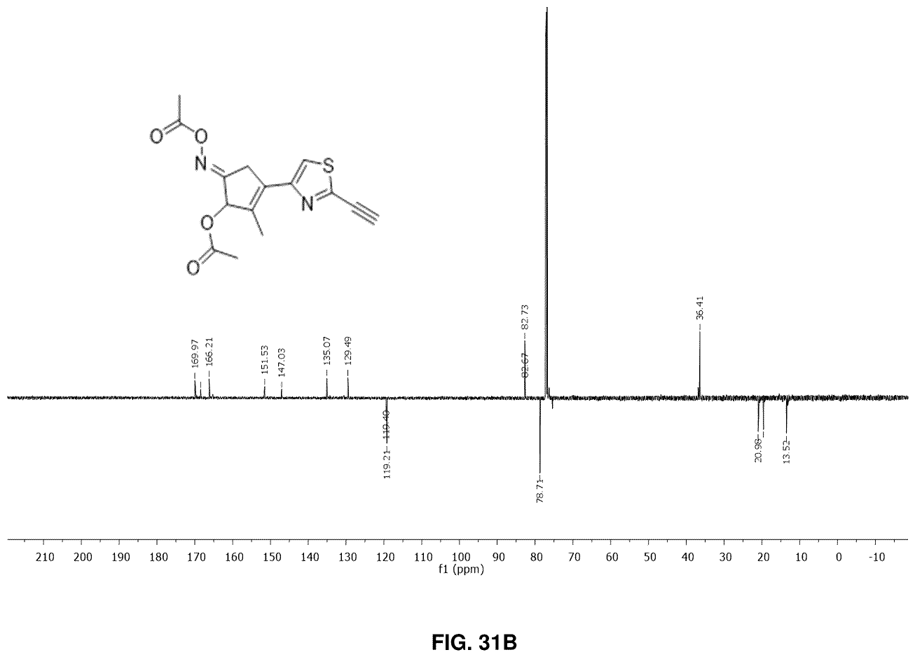

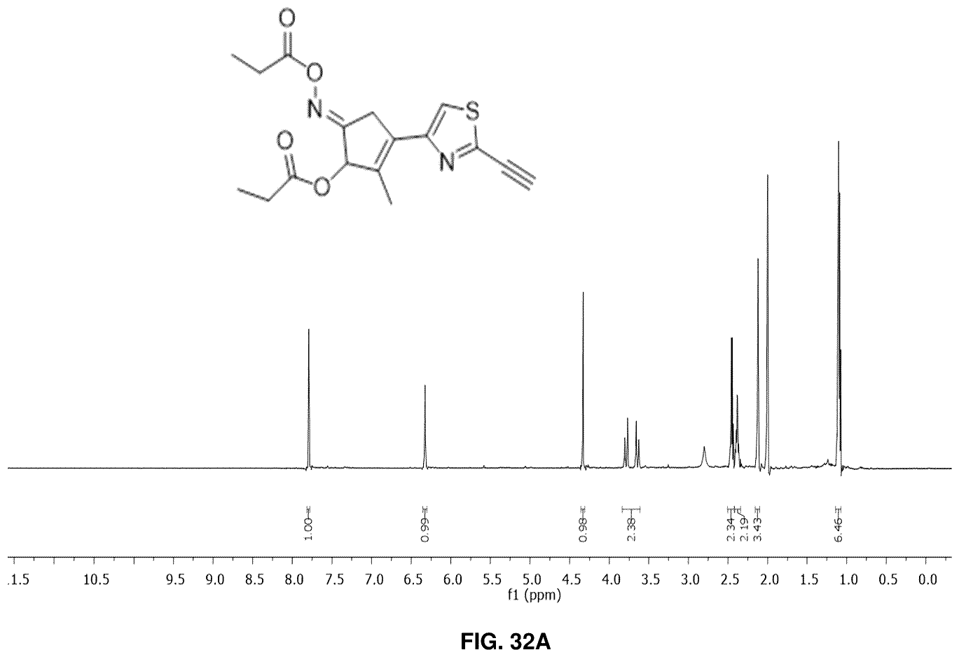

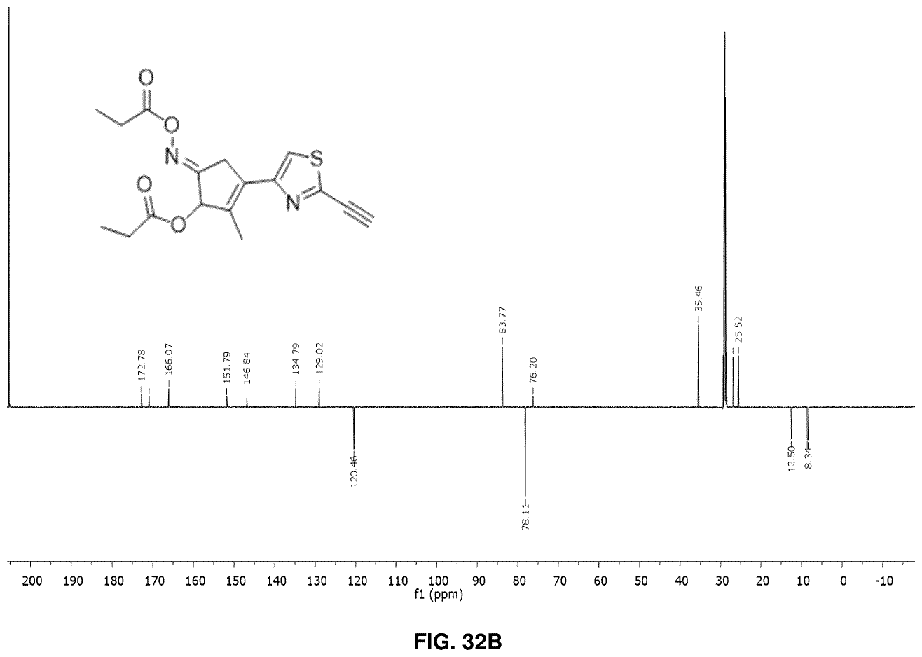

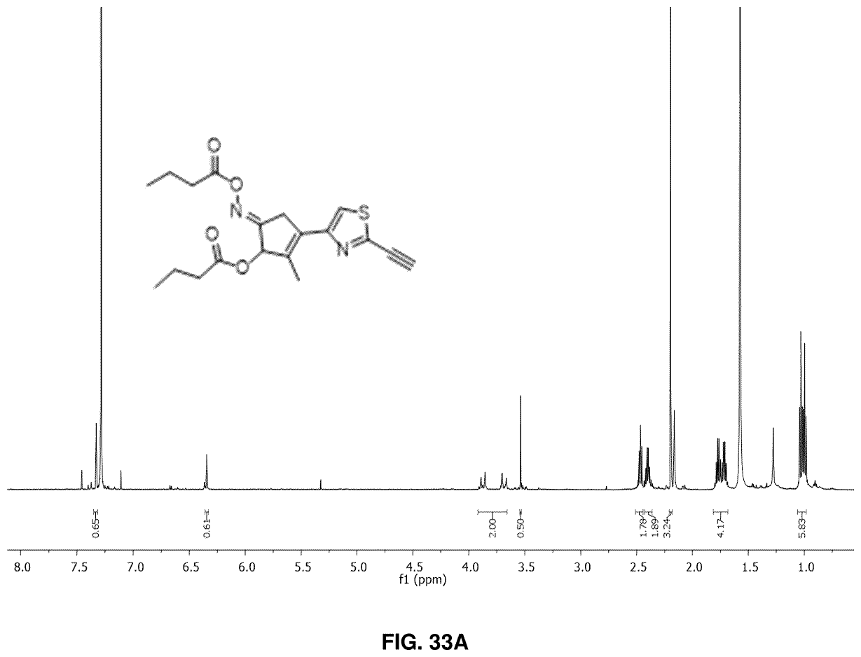

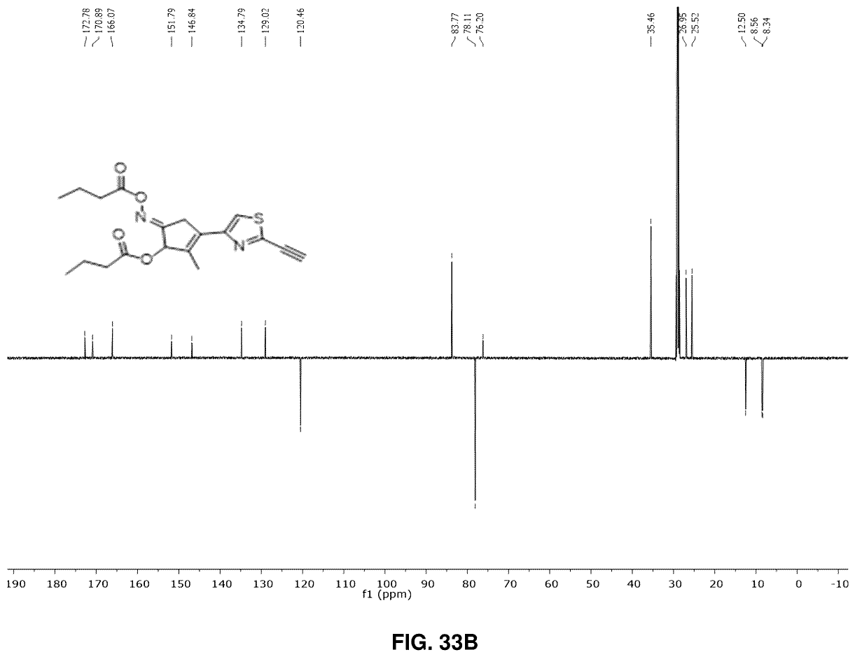

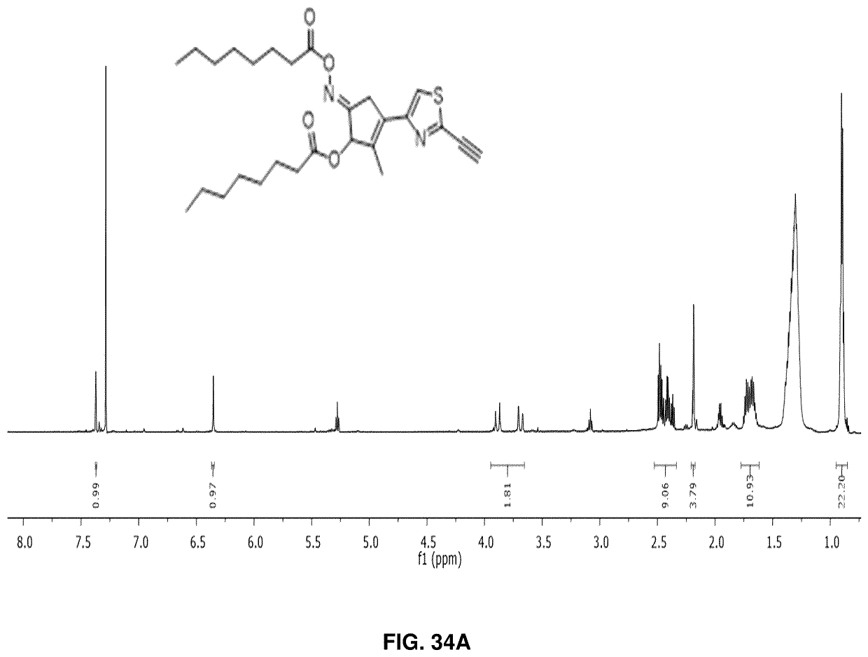

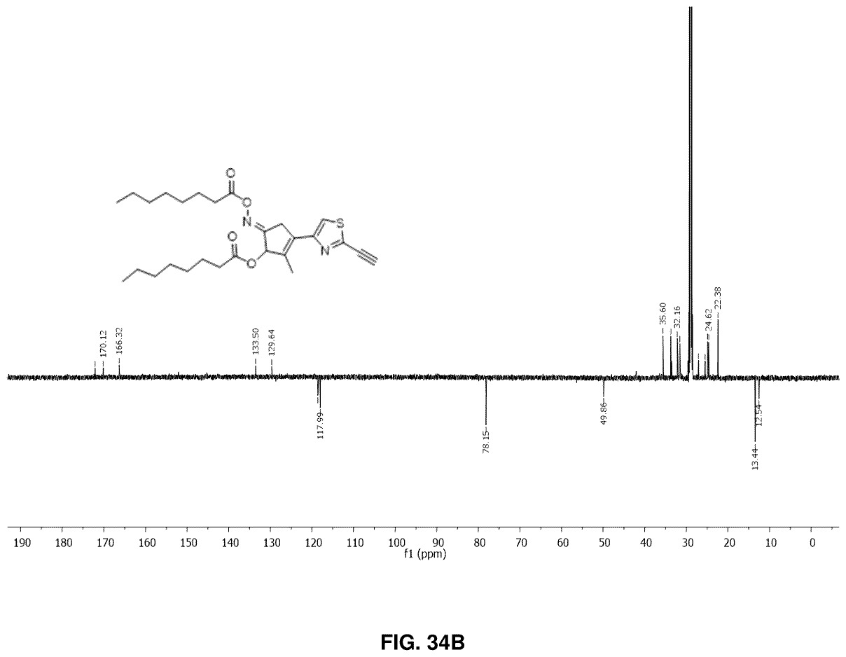

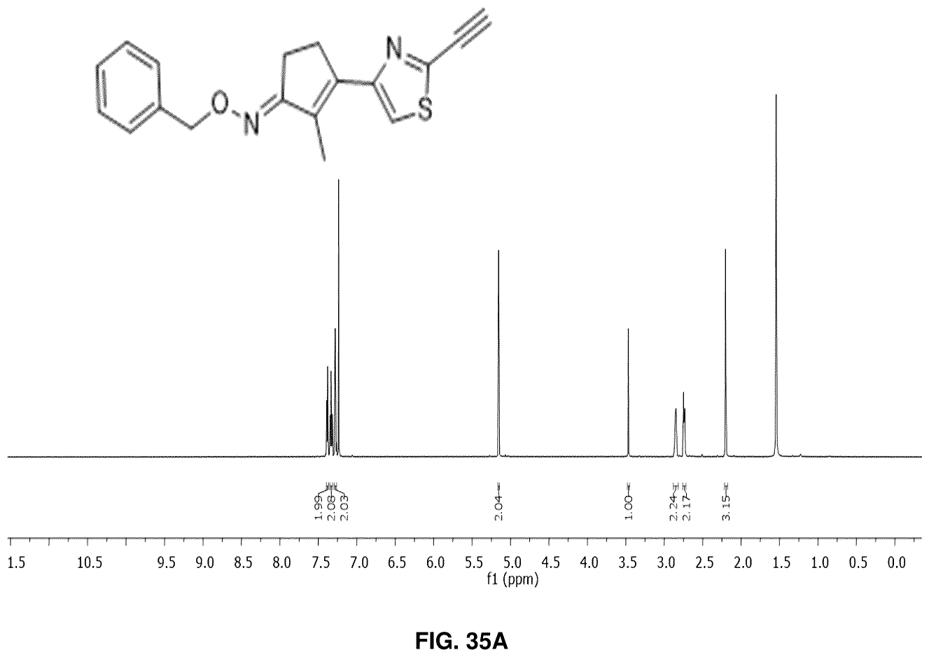

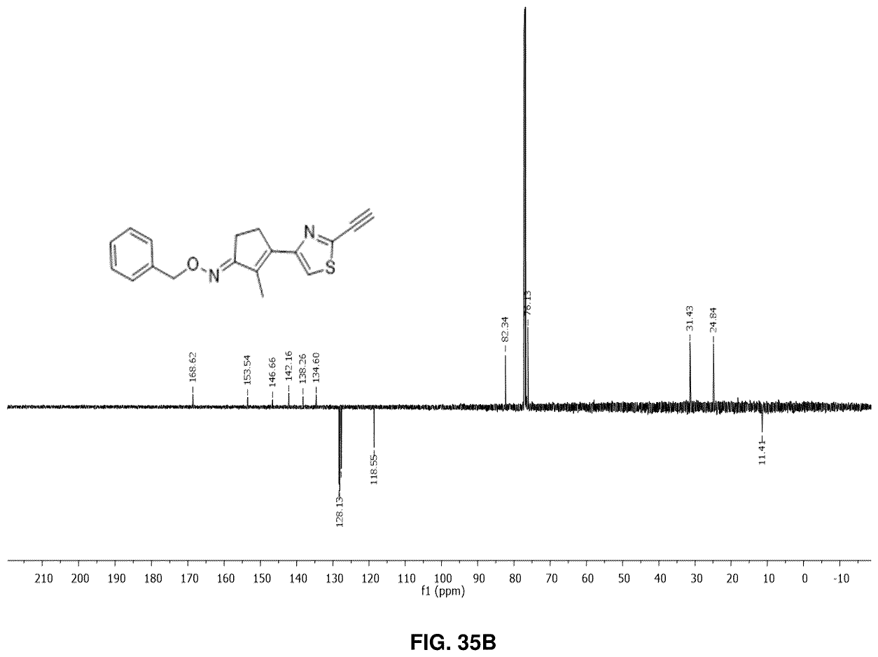

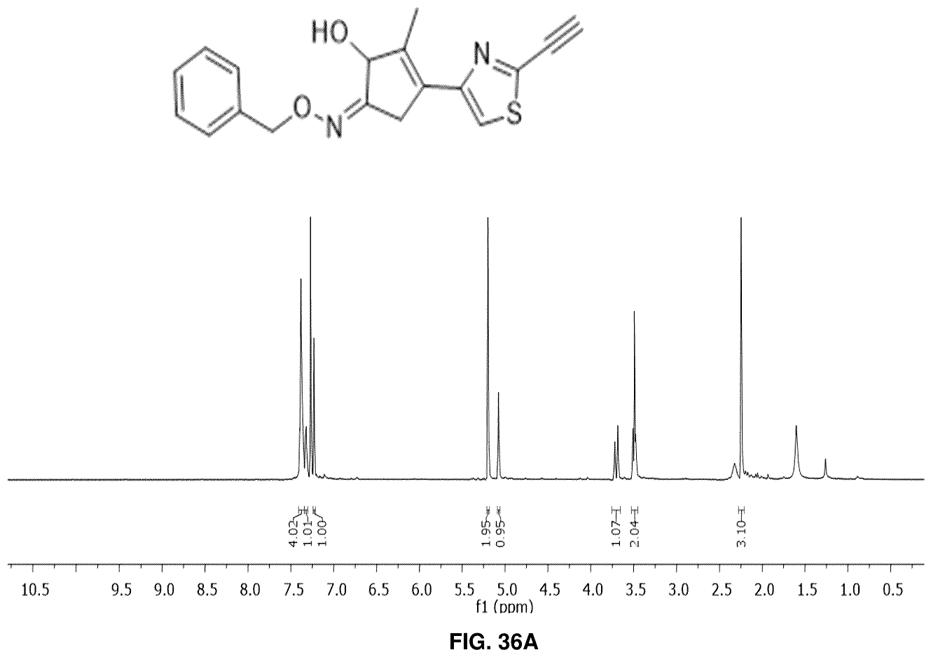

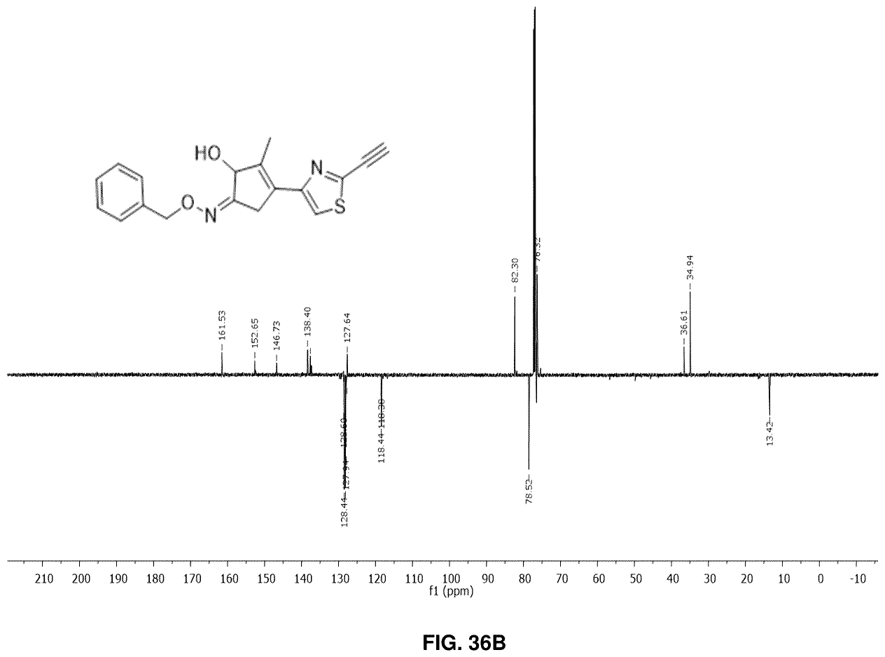

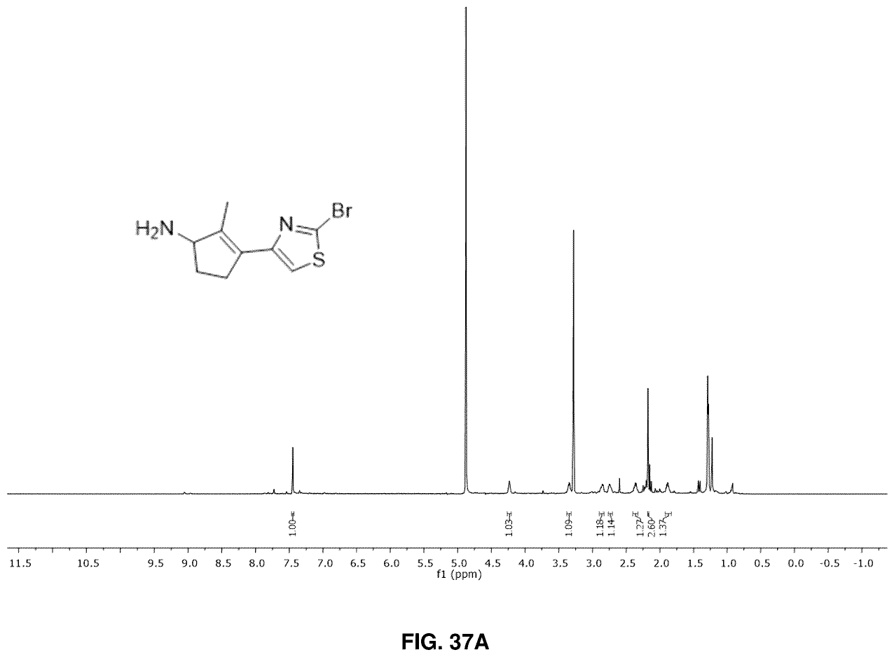

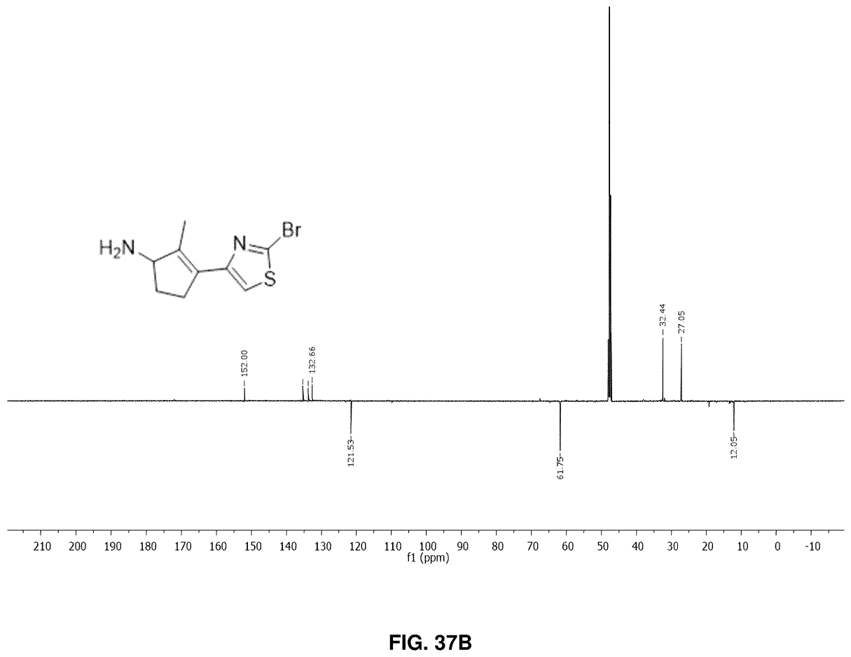

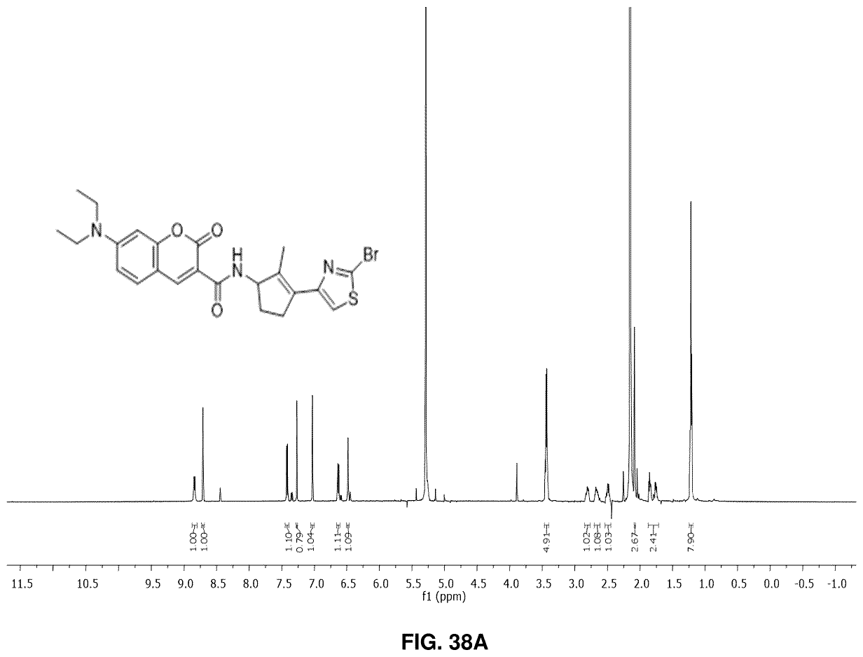

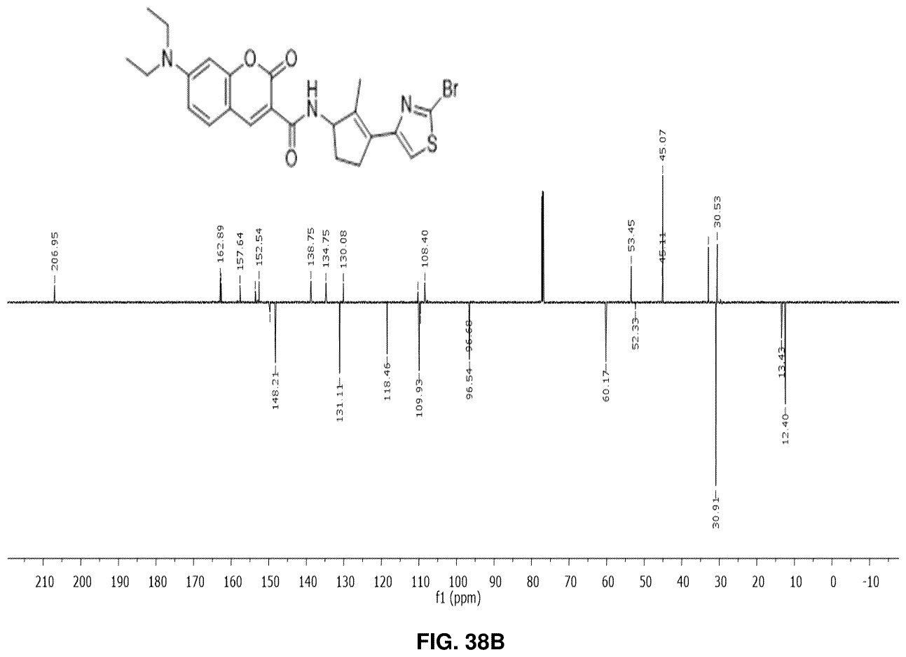

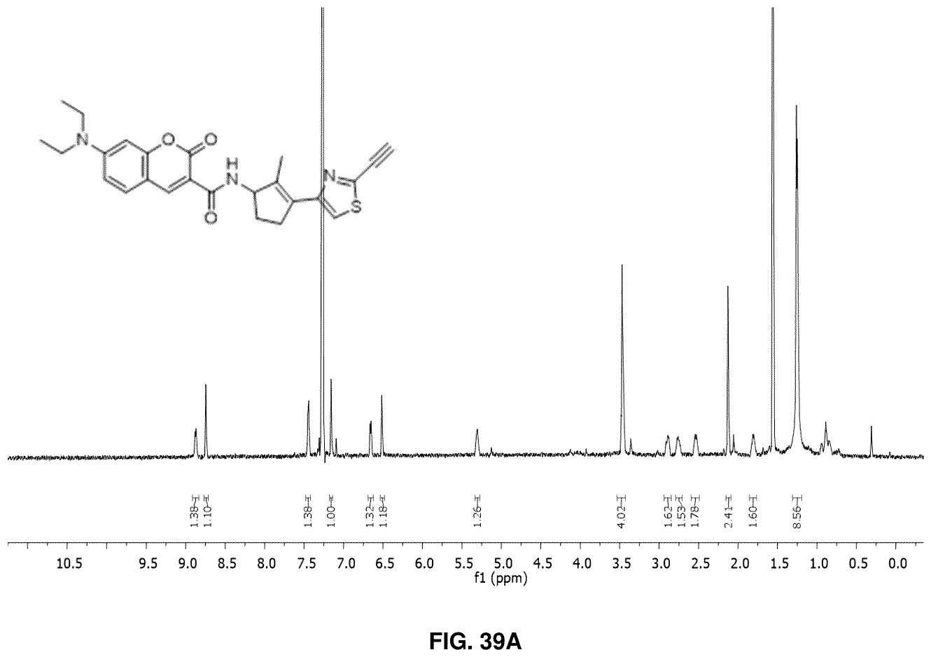

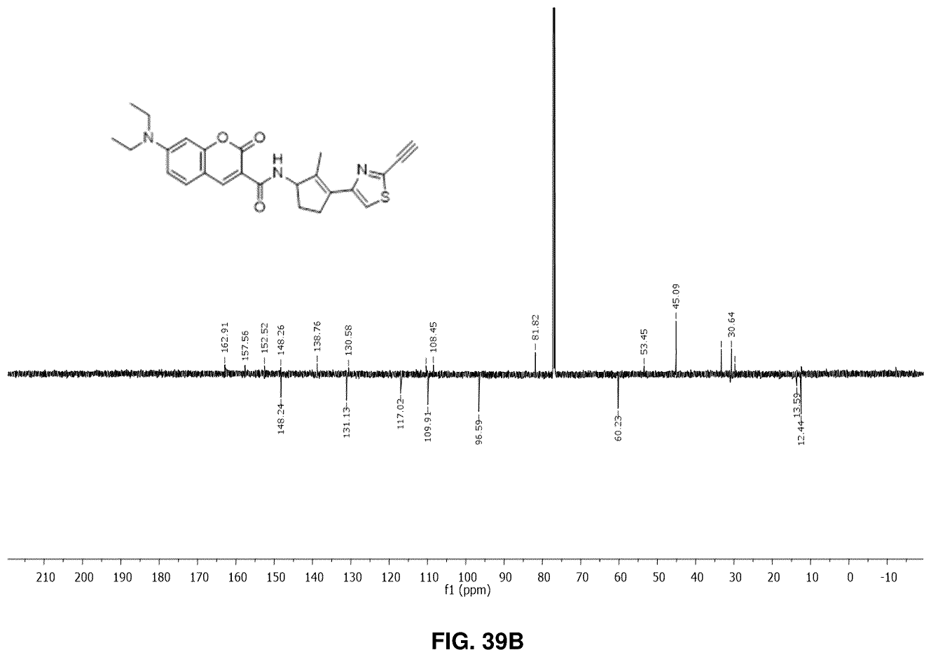

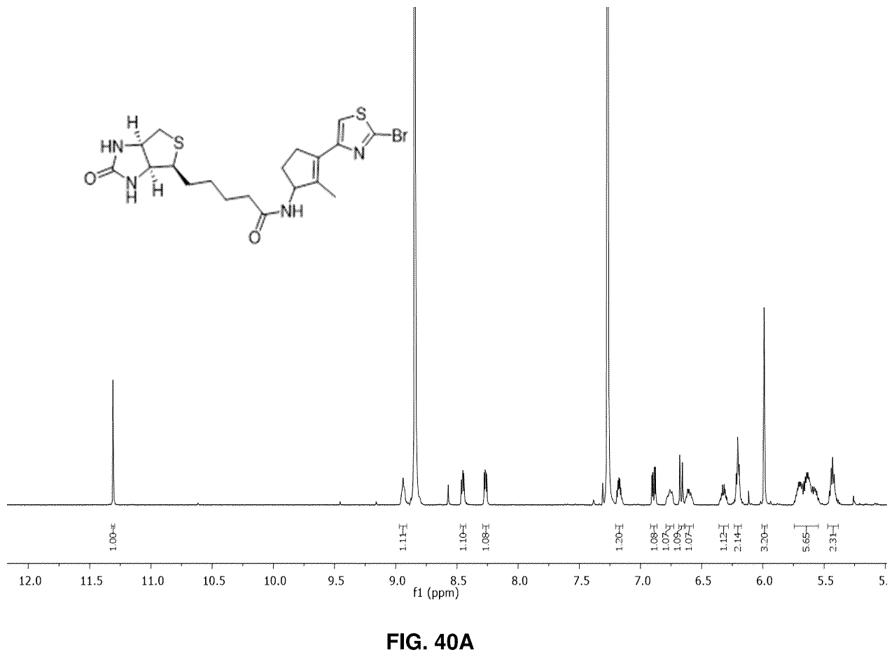

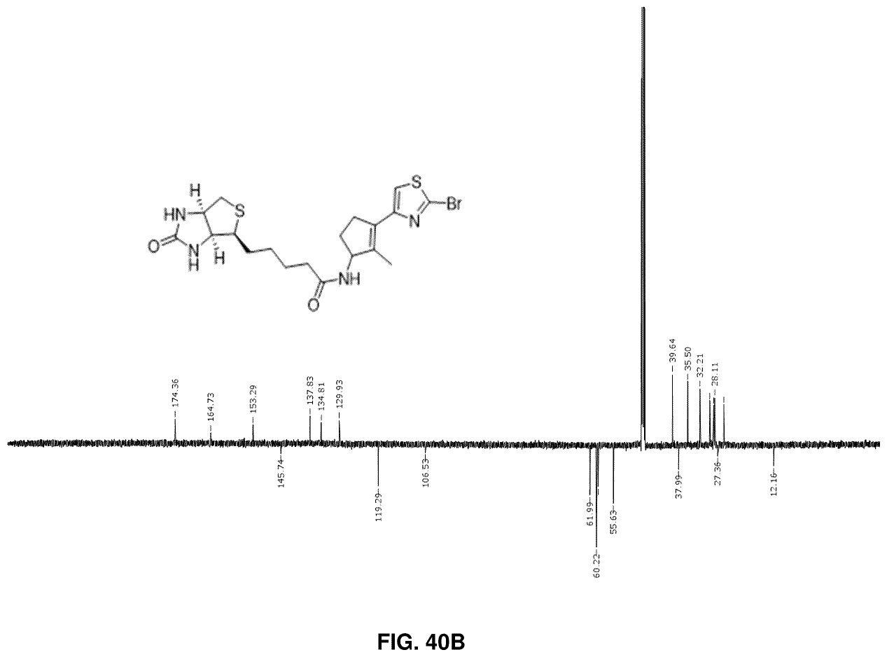

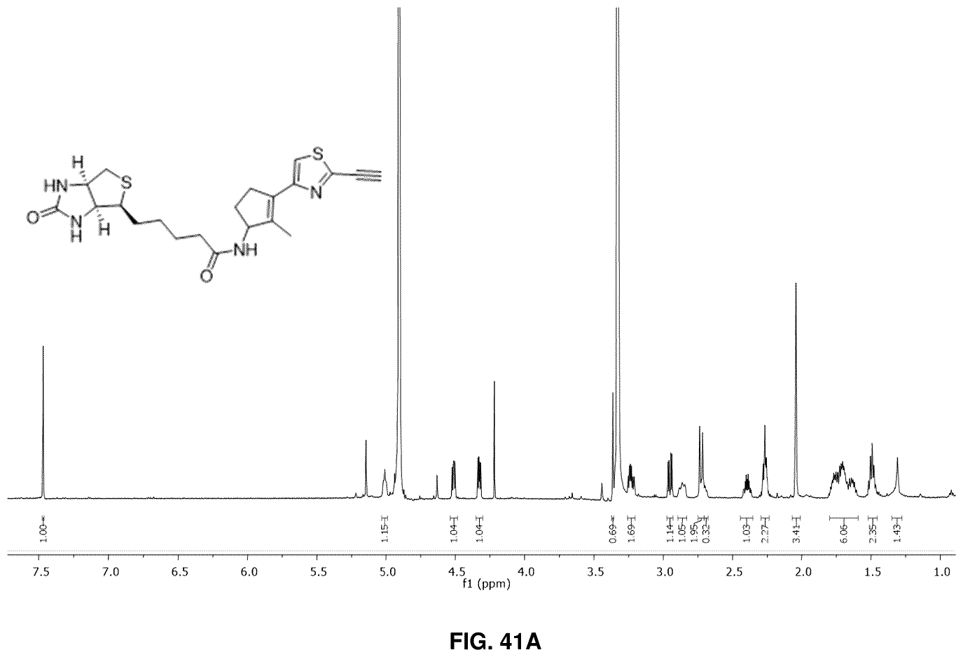

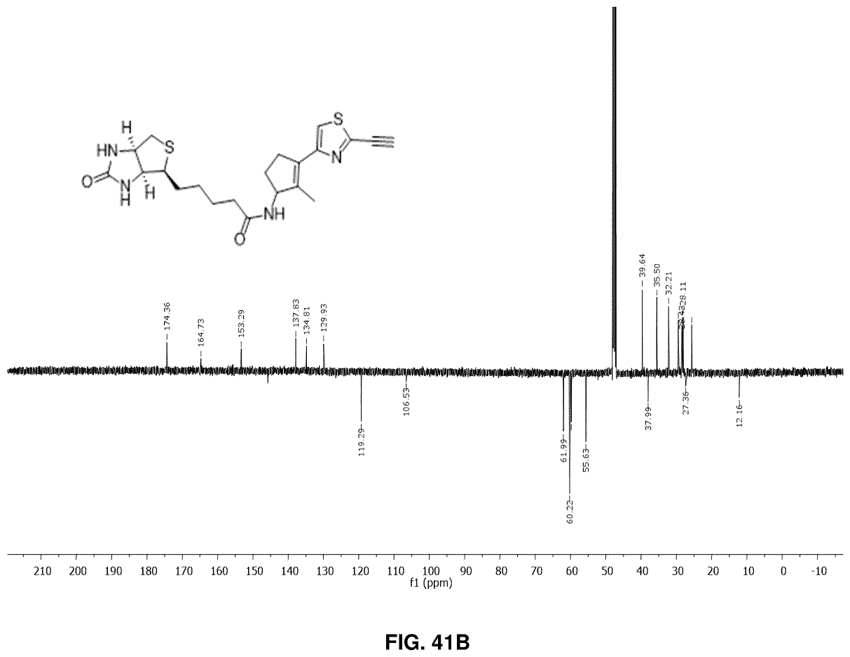

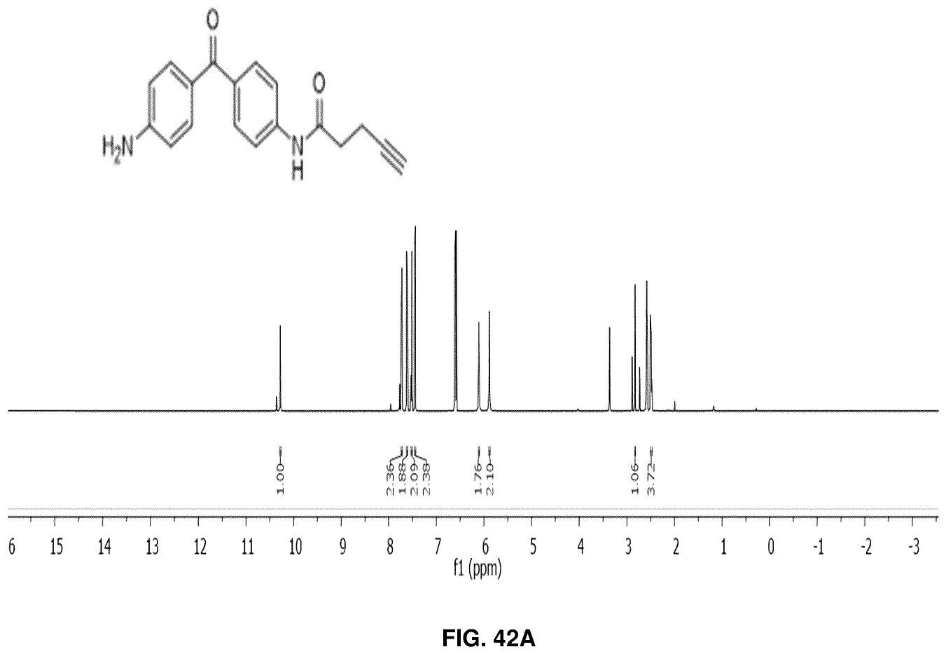

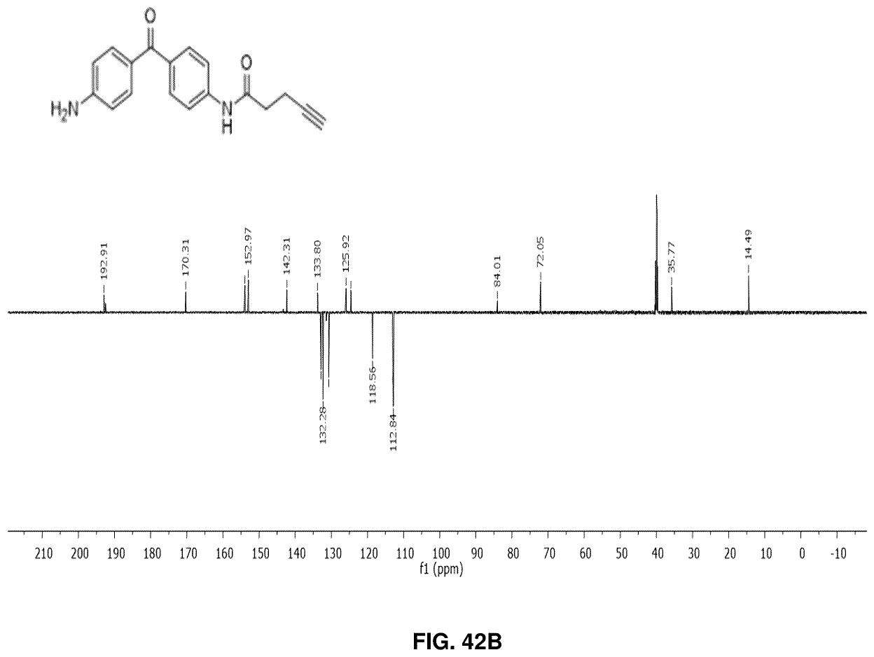

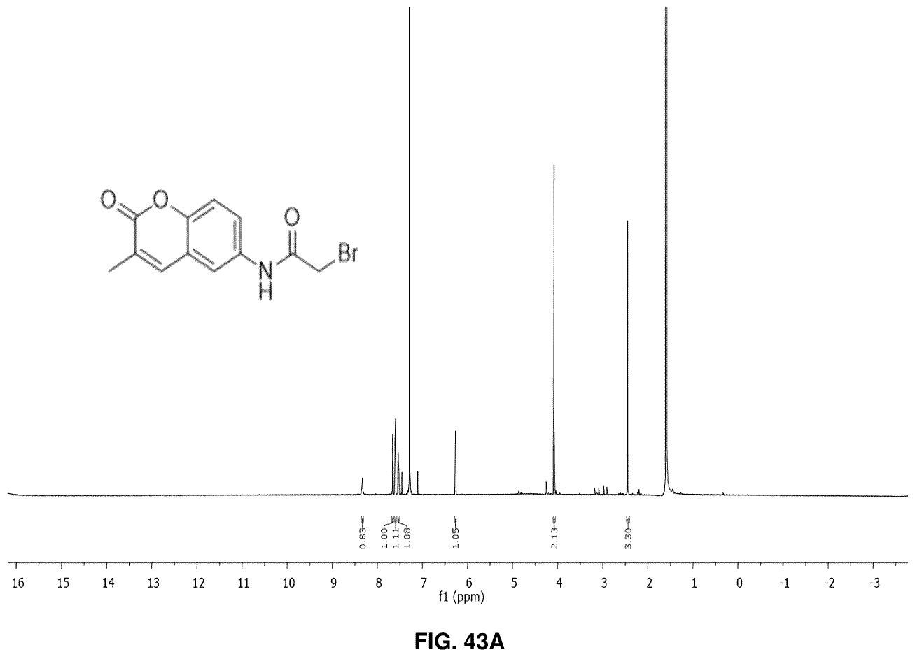

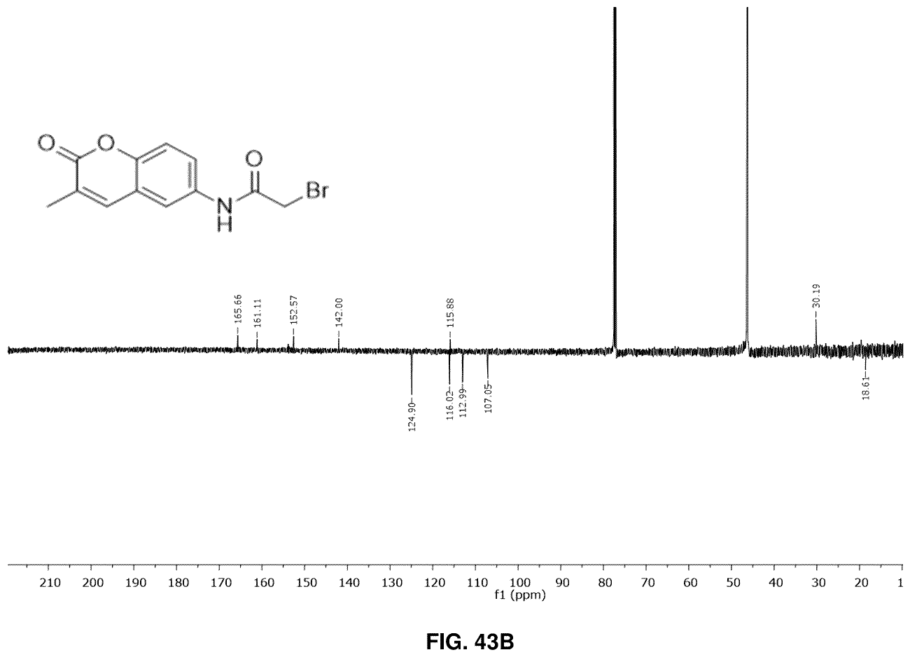

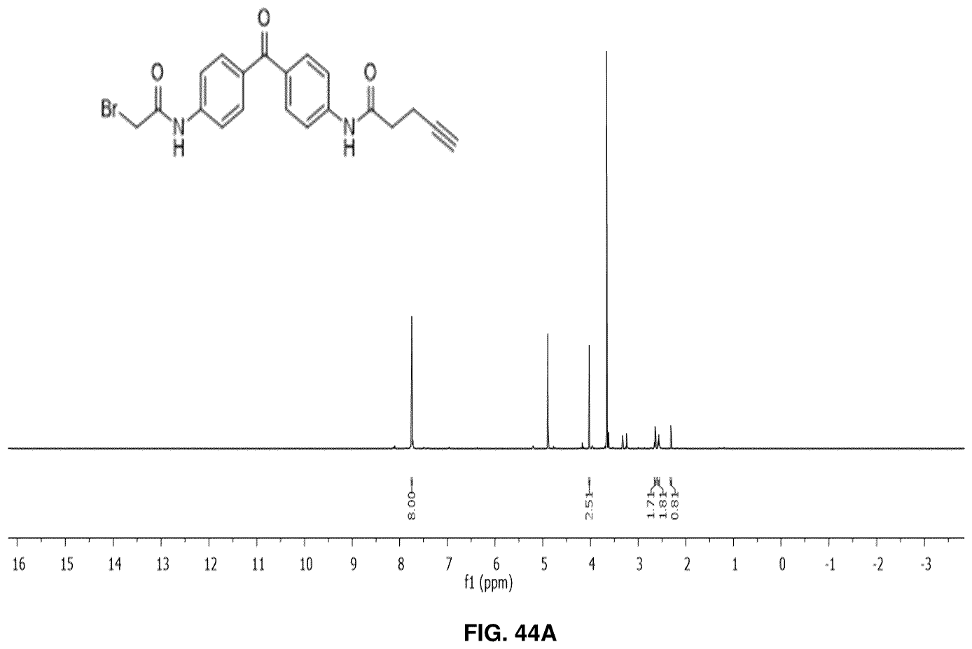

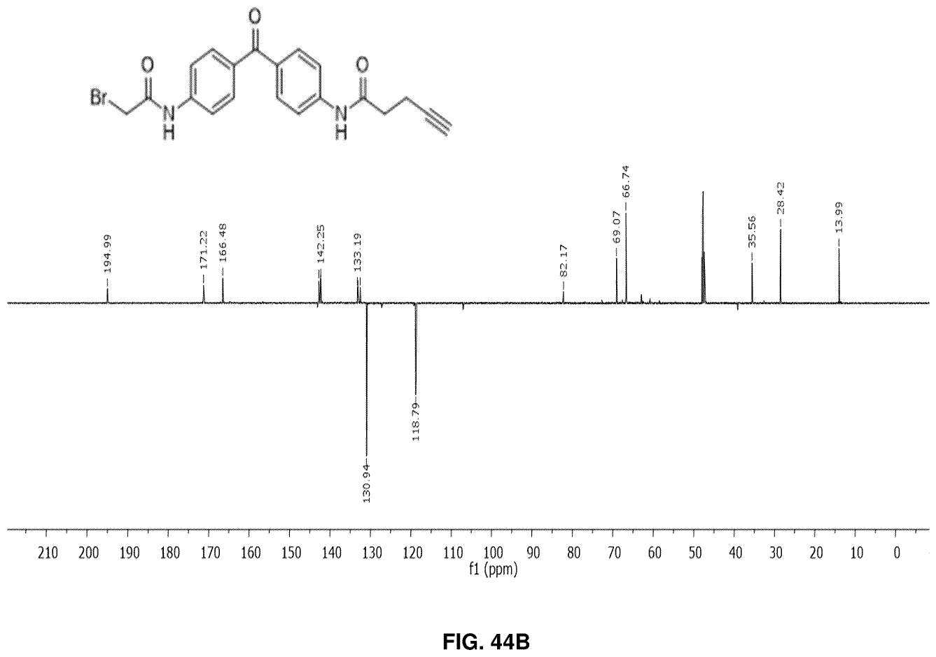

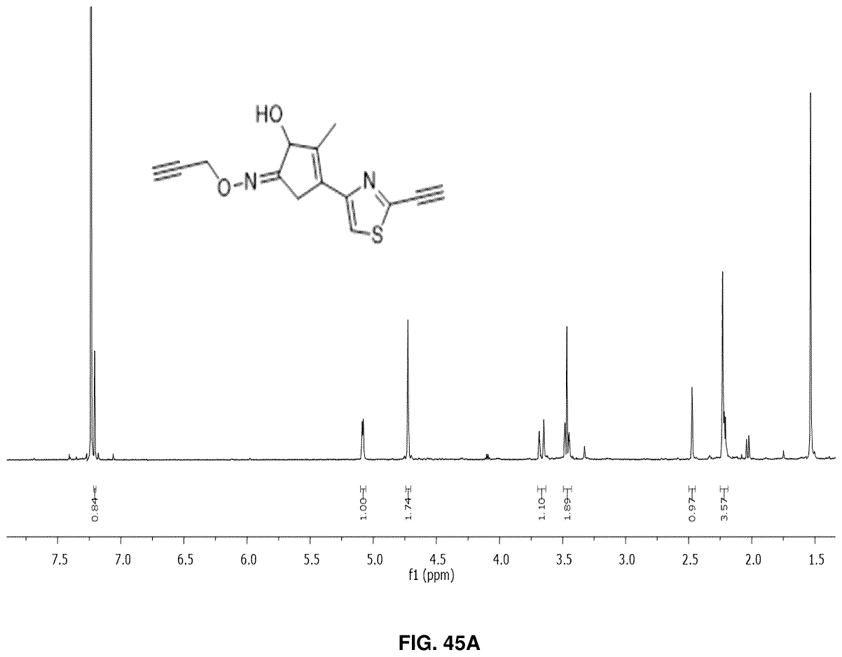

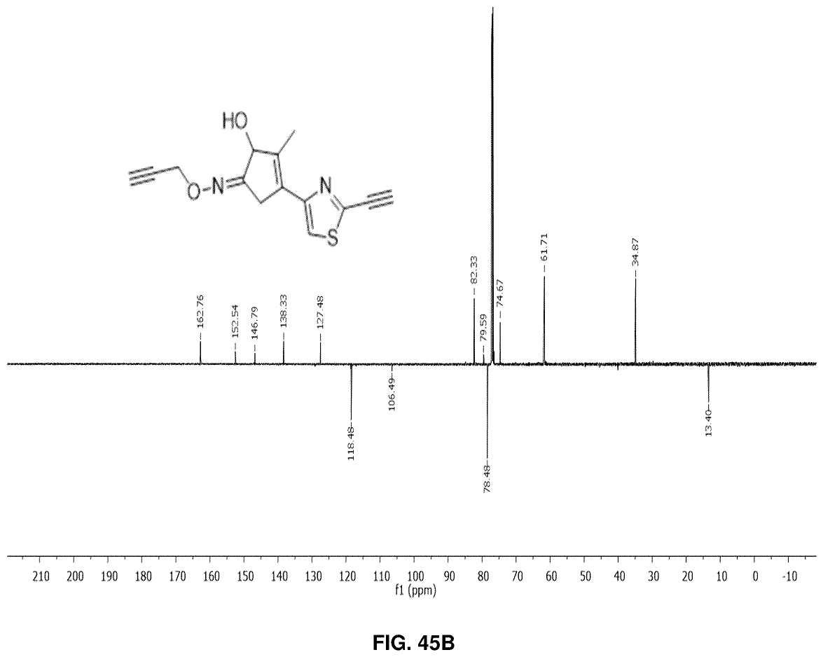

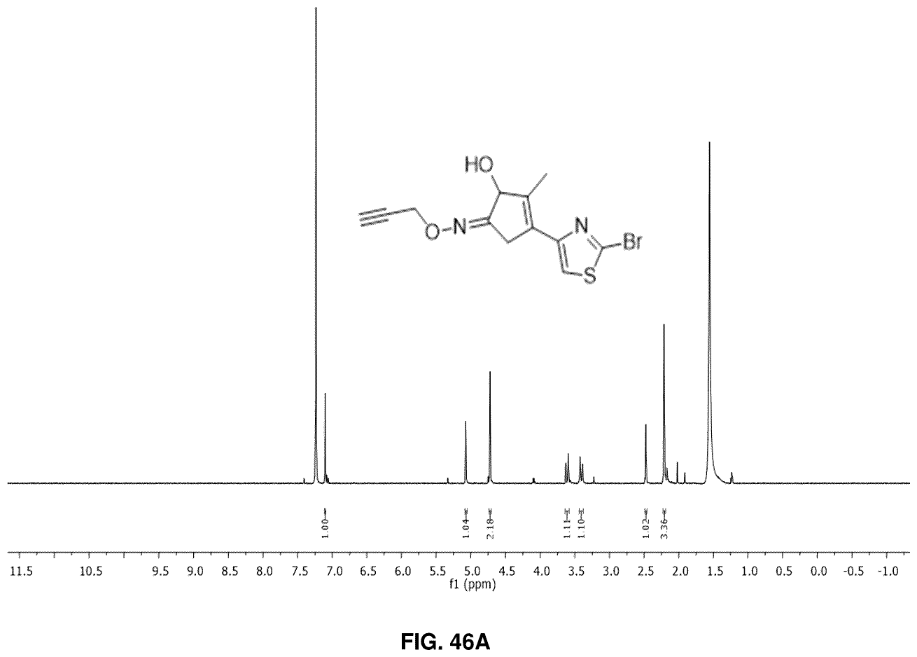

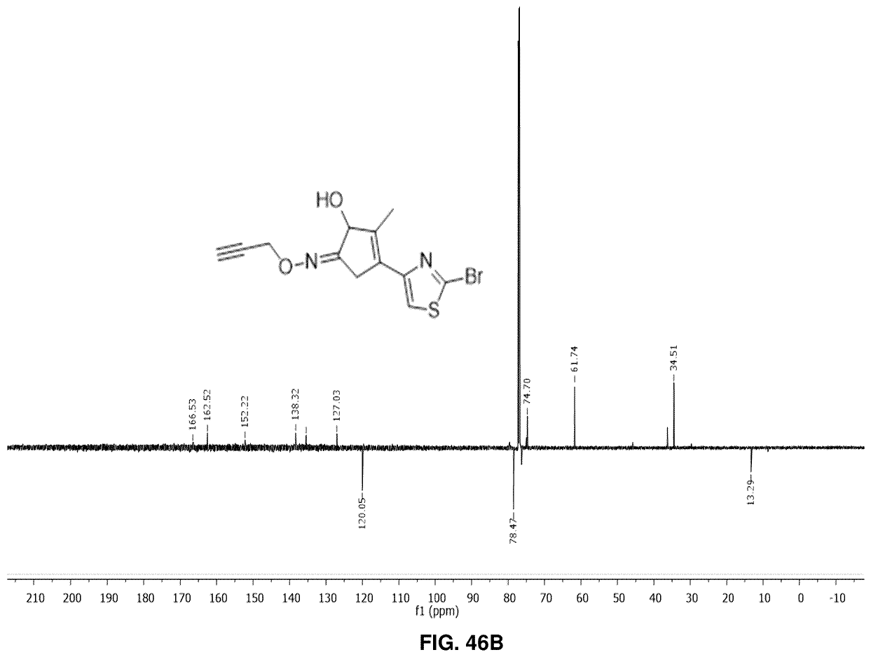

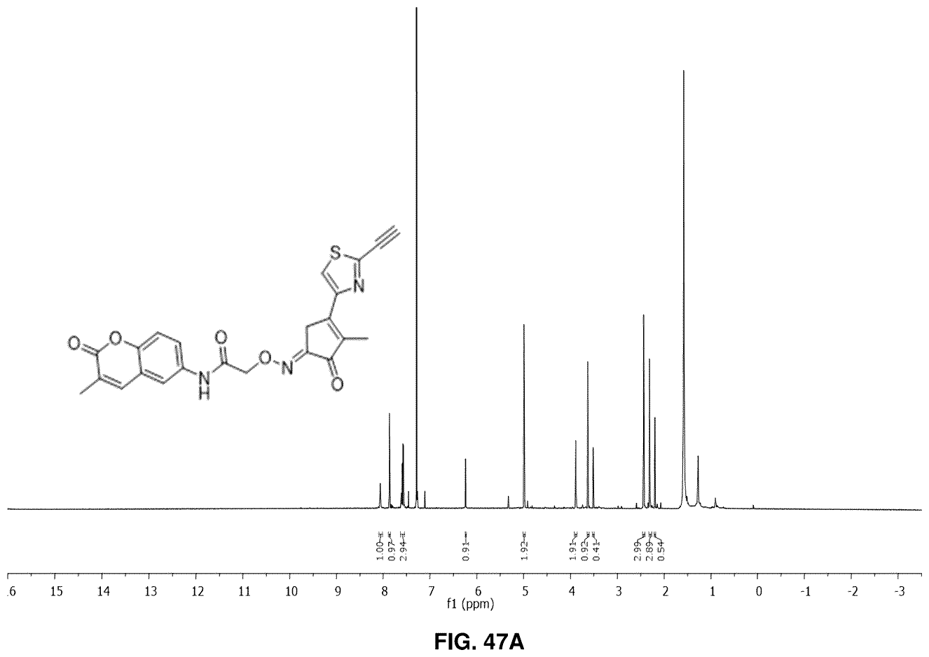

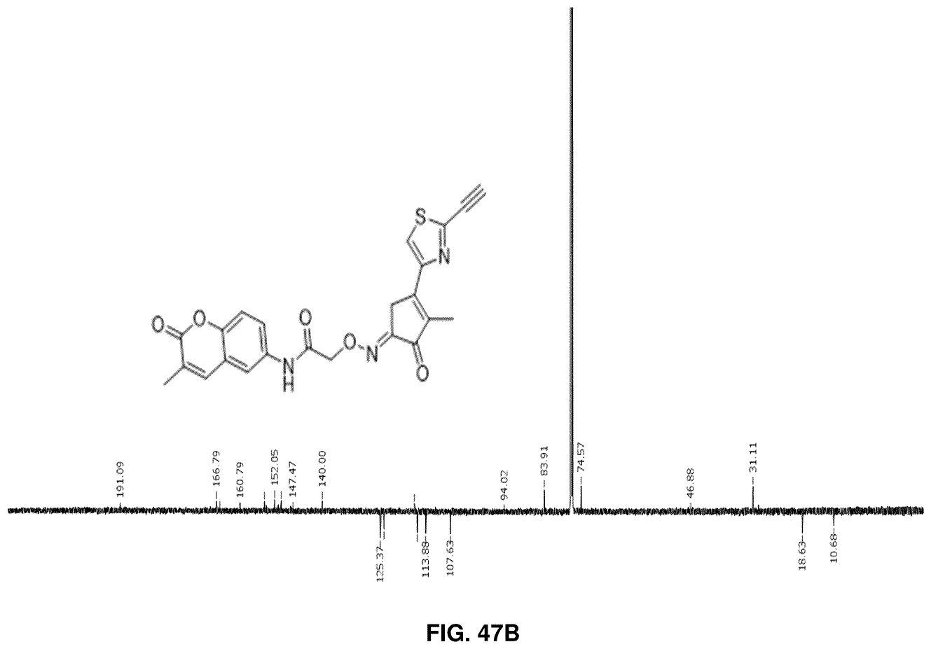

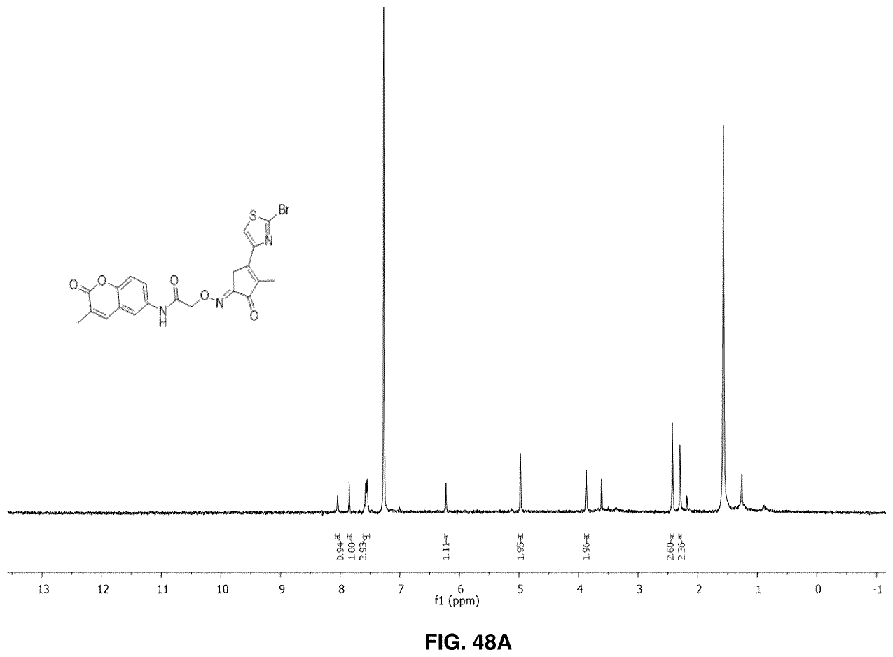

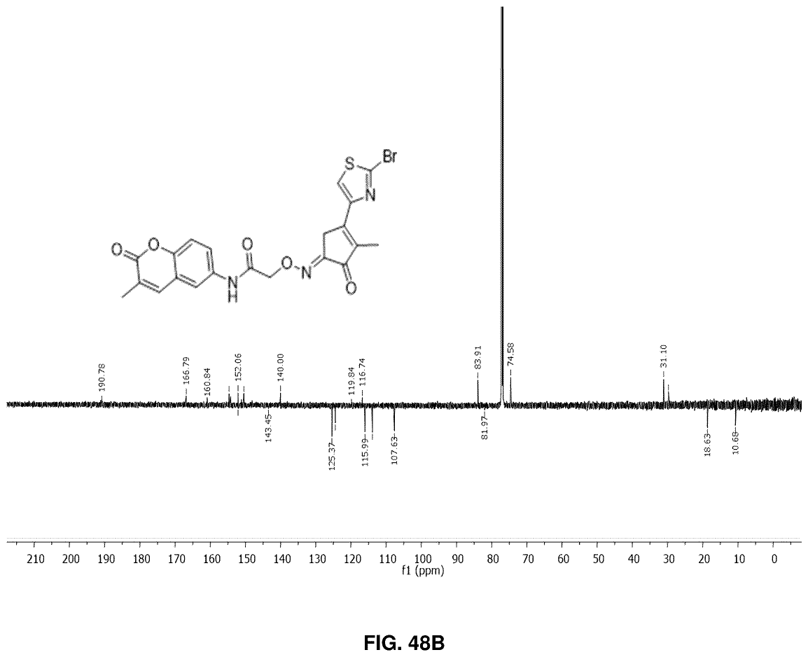

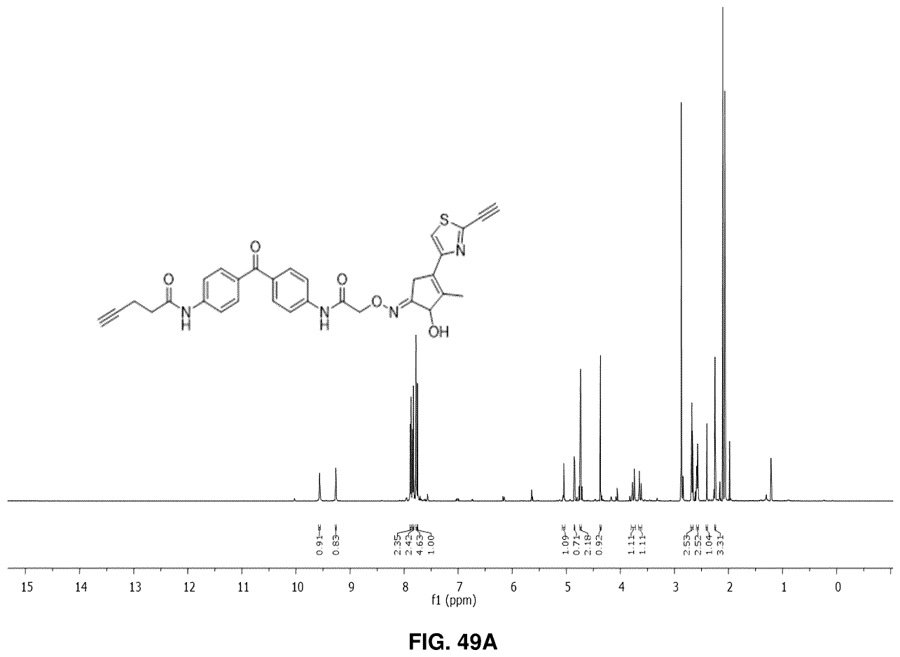

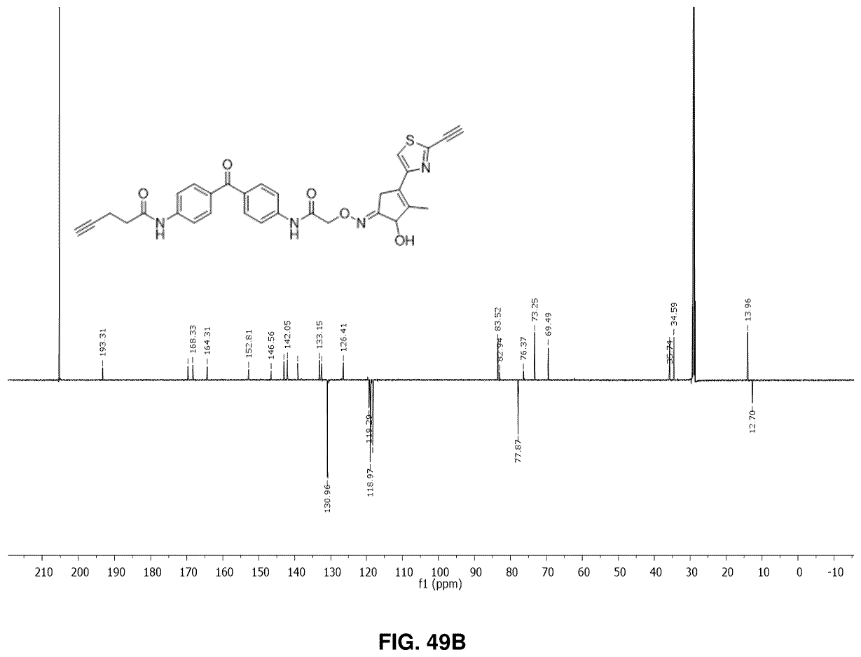

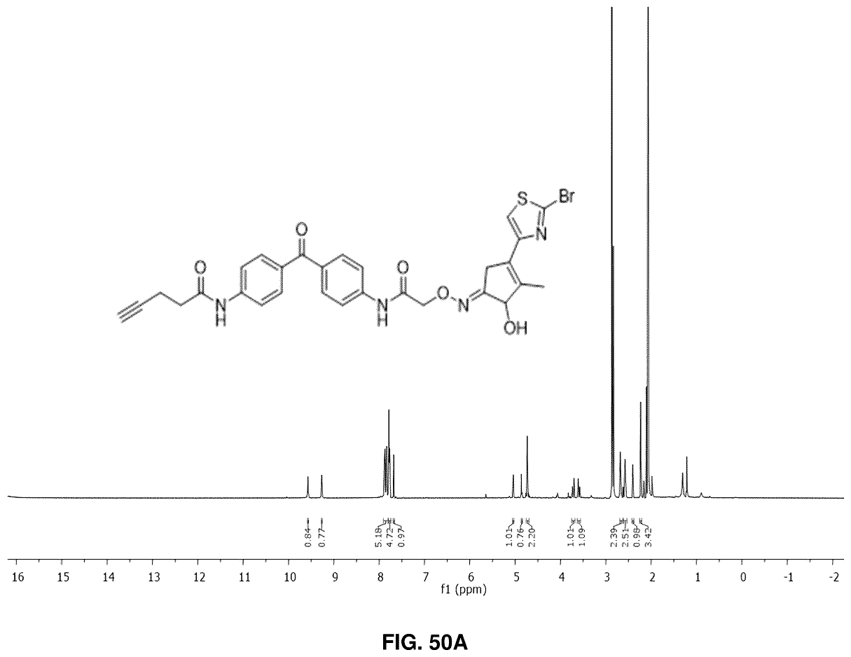

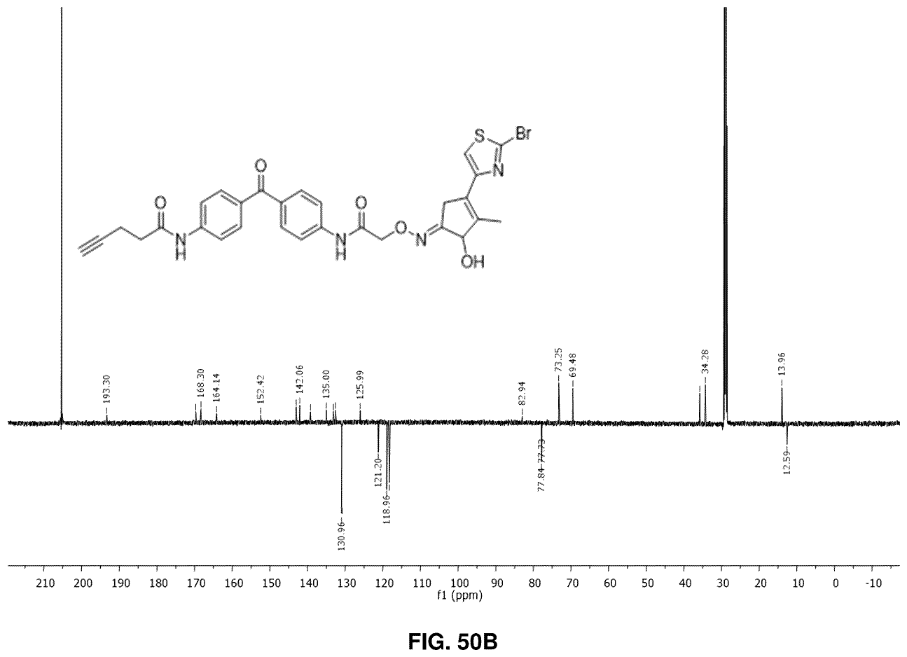

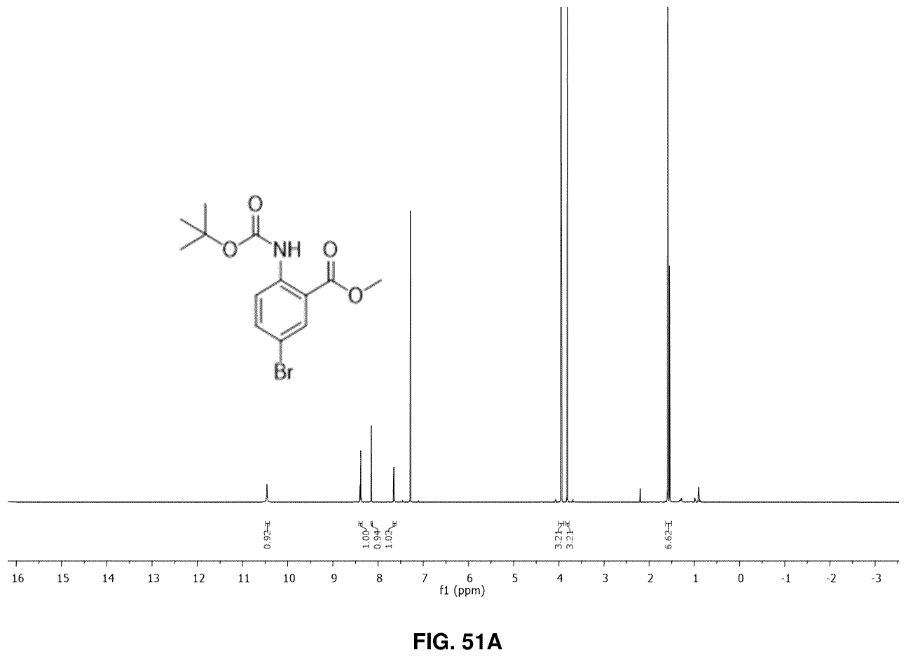

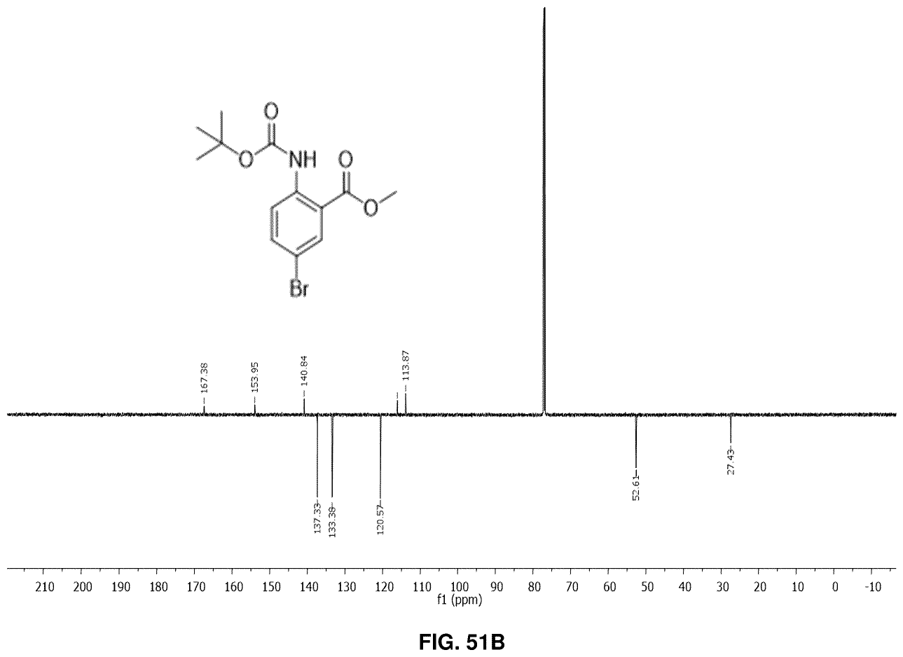

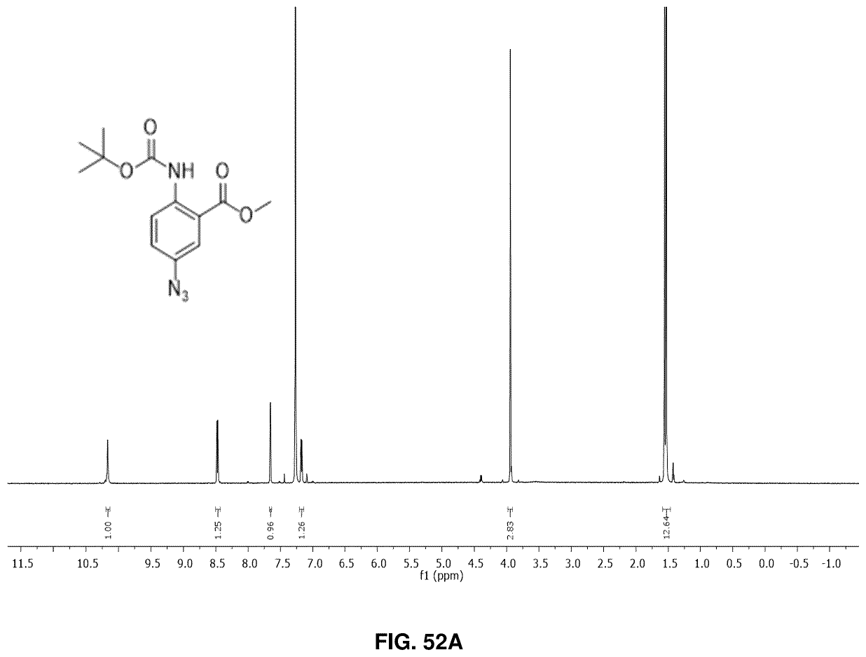

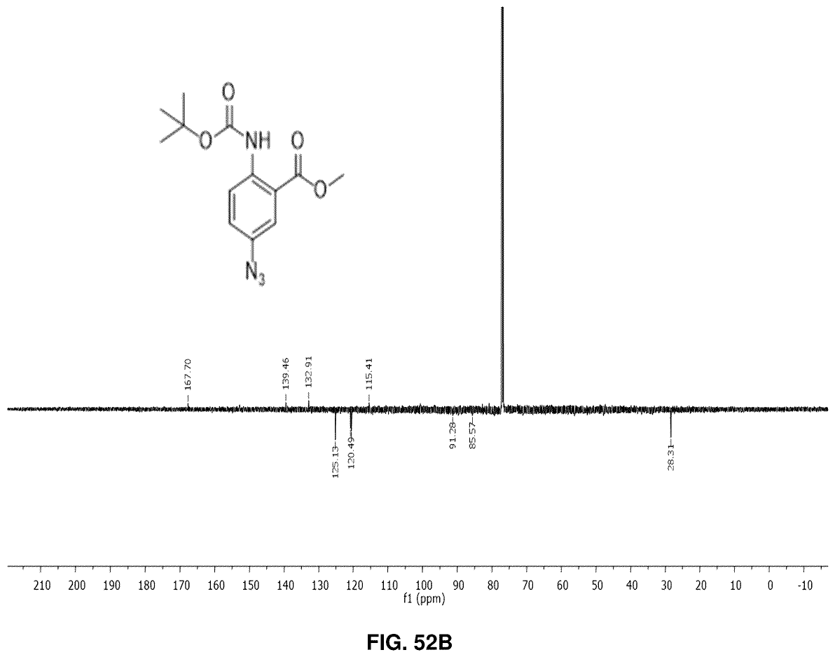

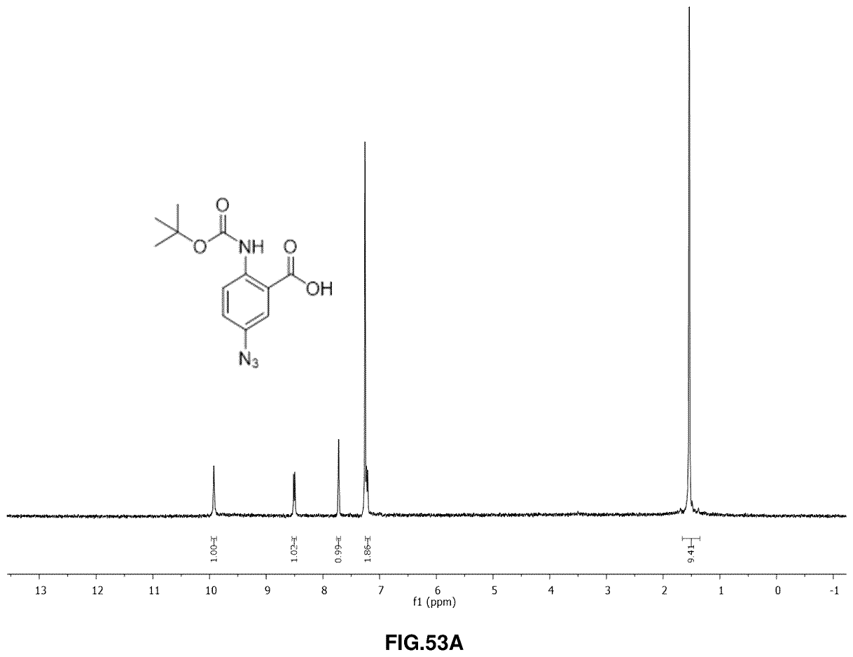

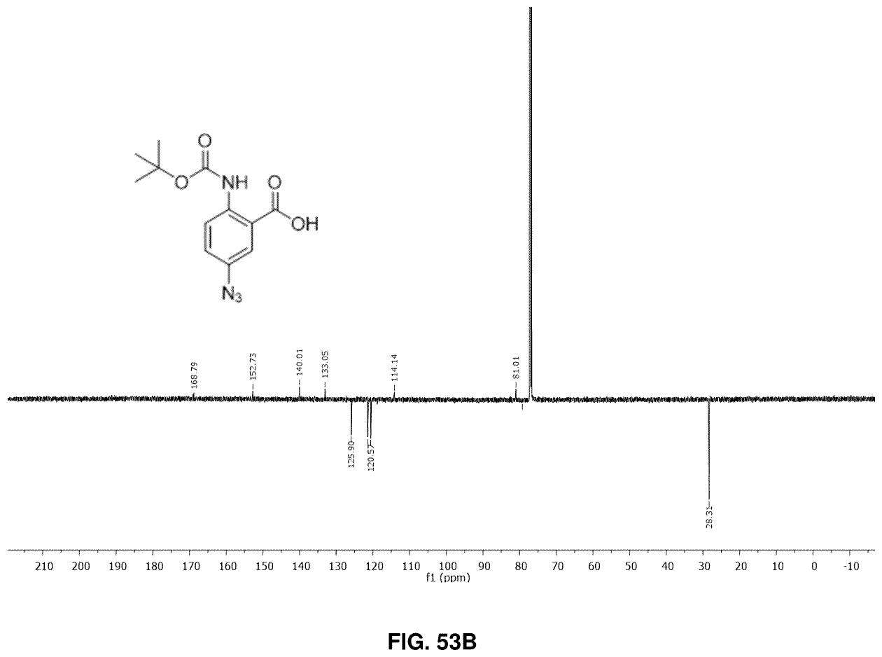

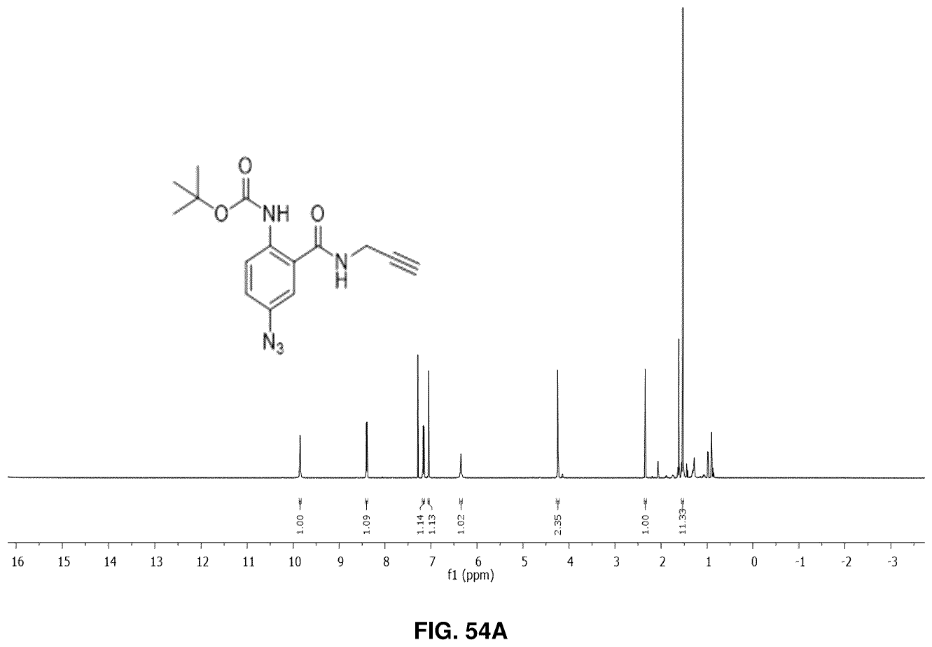

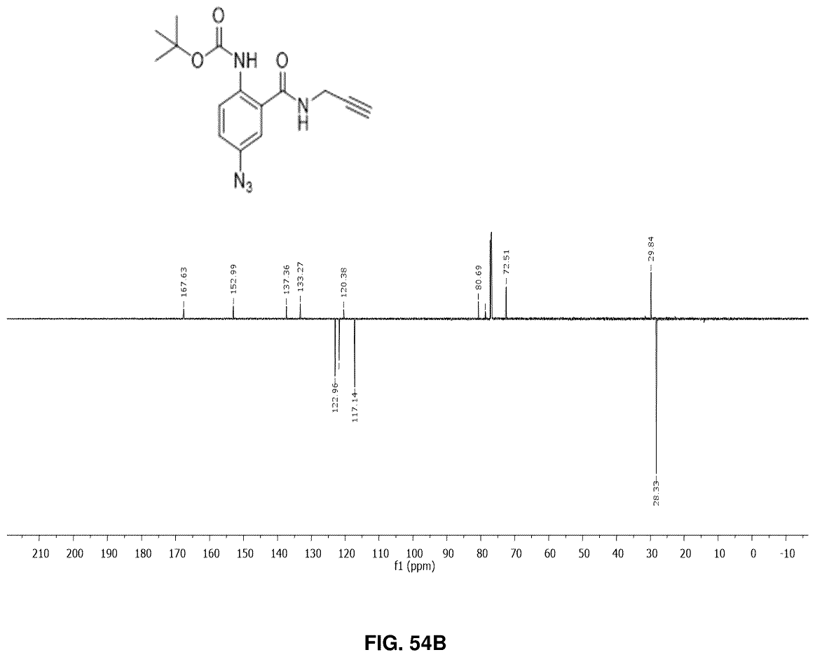

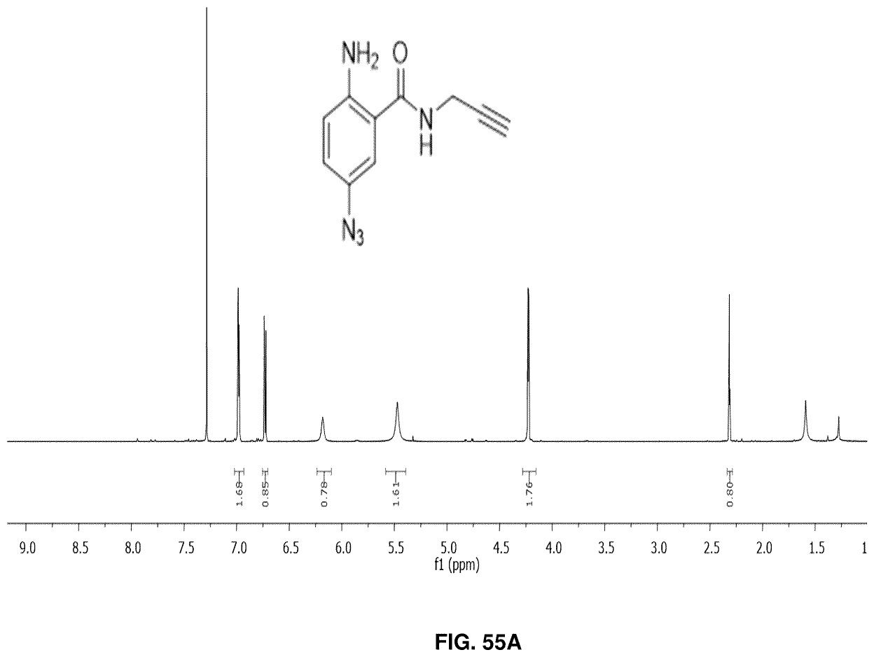

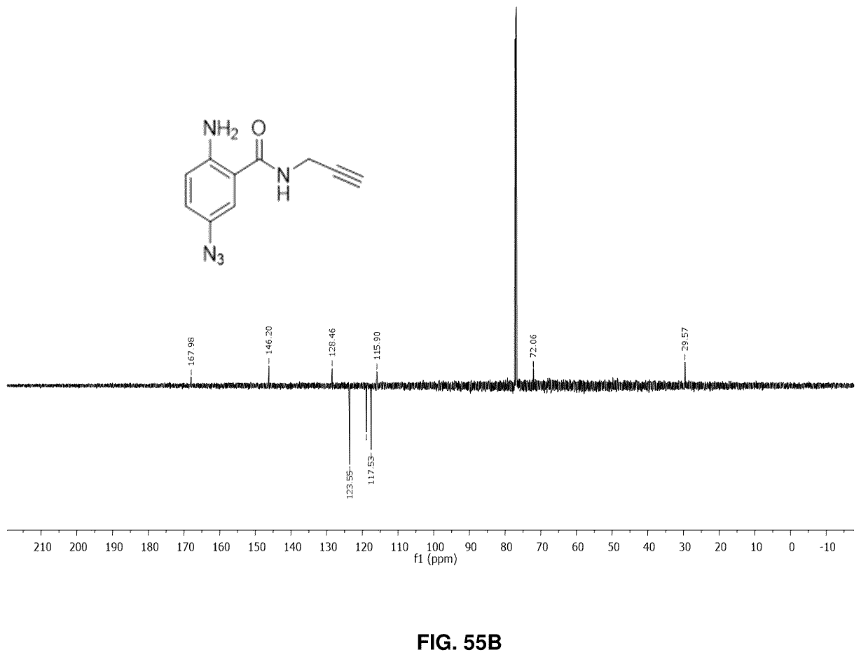

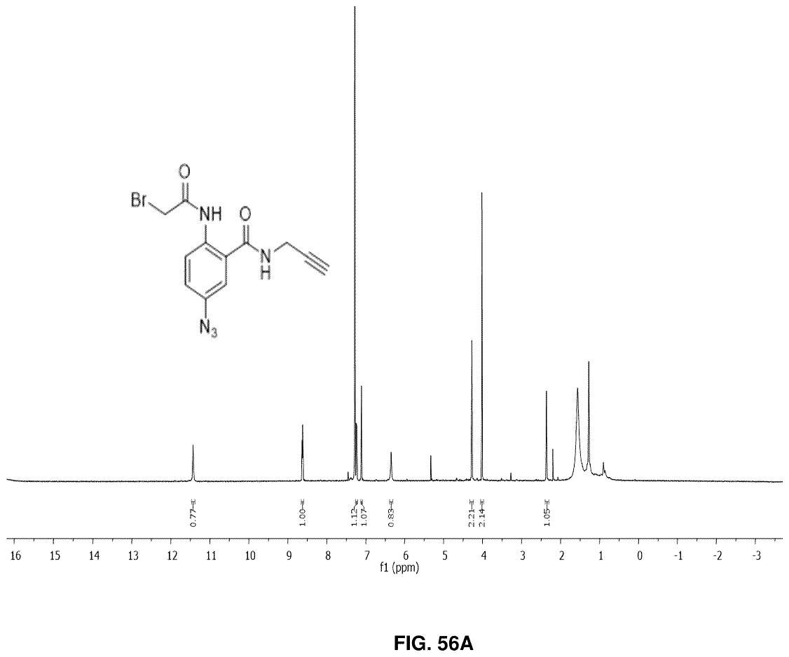

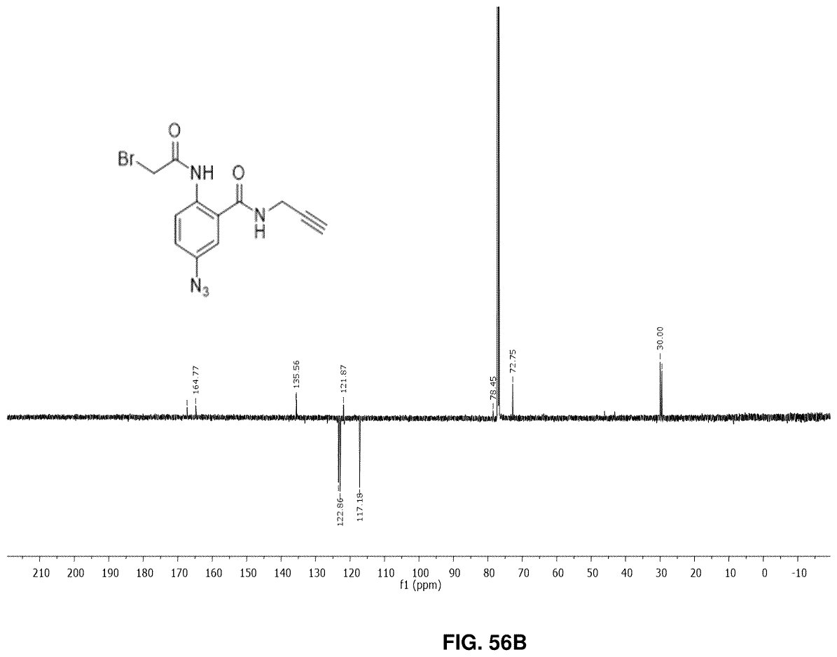

The IC₅₀ values of compounds 20a, 20b, 20c, and 20e compared to CETZOLE-1 against NCI-H522 are shown graphically in FIG. 9A.

The oxime O-benzyl ethers 21 and 23 were active against most of the tested cell lines, but less cytotoxic on NARF2. The IC₅₀ values of these two analogs in other cell lines are in the single digit micromolar range. Therefore, these analogs are suitable templates for designing the probe molecules. Control CETZOLE 1 proved to be less cytotoxic compared to the other control RSL3 and also the prodrug analogs 20a-20c across tested cell lines.

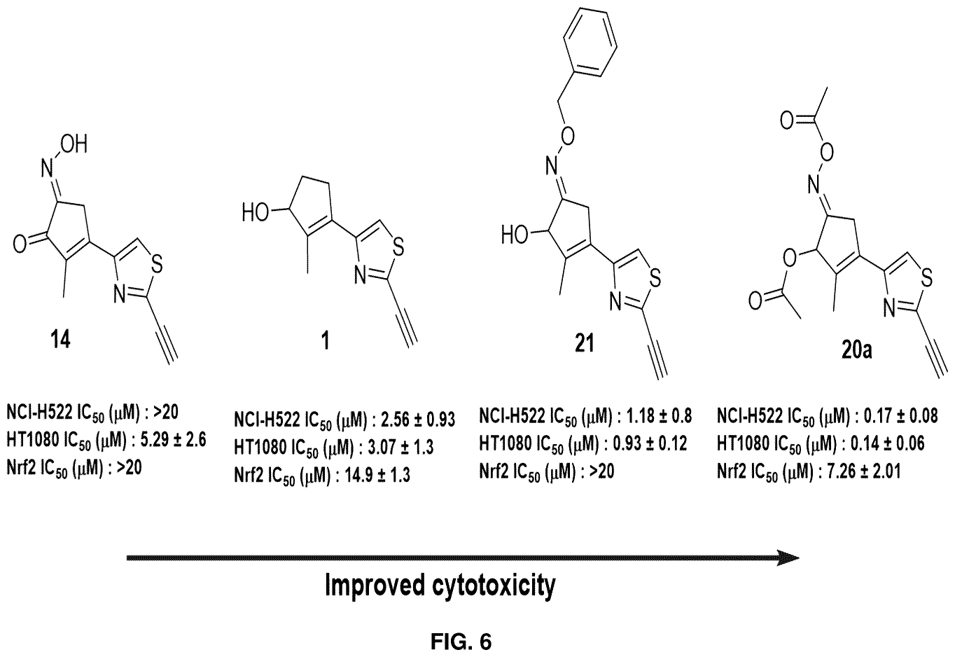

Taken together, the results from the cytotoxicity screening (summarized in FIG. 6) show that converting the ketone at the allylic position to a hydroxy group enhances activity. Installing an O-benzyl oxime group, either at the allylic (23) or the homo-allylic (21) position, contributes to cytotoxicity. Converting oxime alcohol to a prodrug gives the most cytotoxic compounds. There is an overall drop in activity against NCI-1-H522 cells with overexpressed SLC7A11. This indicates that SLC7A11 is a possible protein target since its overexpression enhances cell survival. However, a similar drop in activity was also observed with RSL3, which is a GPX4 inhibitor and is not known to inhibit SLC7A11. Without wishing to be bound by theory, it is believed that increased function of the X_{c}⁻ antiporter may elevate glutathione to protect against ferroptosis induced in multiple ways. Consistent with ROS-dependent ferroptotic cell death, oxidation-resistant NARF2 cells were found to be highly tolerant to all CETZOLE analogues.

Next, live cell imaging was conducted to study the death pattern of NCI-H522 cells treated with 10 μM concentration of controls and analogs. The results are shown in FIGS. 10A-10C. A montage of cell images was analyzed with Kaplan-Meier plot, which showed analog 20a to have a cytotoxic duration and killing pattern similar to that of RSL3 with 50% cell death taking place in 3-4 hours after treatment (FIG. 10A). CETZOLE 1 and analog 23 induce cytotoxicity slower, killing 50% of cells in 4-5 hours after treatment.

The live cell montage images in FIG. 10A show notable ferroptosis-related morphological changes, such as membrane breakage and floating cell debris in the medium. The NCI-H522 cell viability improved overall when cells were co-treated with the radical scavenger liproxstatin-1 (FIG. 10C). Analog 23 gave the highest rescue of 85% viability, whilst 20a showed the lowest gain in cell viability at 60%, yet significant. This shows that cell death is ROS-related. To confirm this, flow-cytometry was performed to quantify lipid peroxidation using redox-active BODIPY-C11 (FIGS. 11A-11E).

Similar to CETZOLE 1, analogs 20a-20c and 23 induced lipid peroxidation in NCI-H522 cell population compared to DMSO, as shown by flow cytometry (FIGS. 1A-11E). The total cell counts are represented by the area under the graph. The smaller area under the graph for analogs 20a-20c is due to death of approximately 60% of cells prior to flow-cytometry analysis, showing that these analogs induced faster cell death. The oxime benzyl ether 23 and the reference CETZOLE 1 induced slower cell death as shown by the larger area under graph.

TABLE 2
Half maximal inhibitory concentration (IC₅₀) values
of CETZOLE analogs 20a-20c, 23, and controls CETZOLE
1 and RLS3 against cancer cell lines NCI-H522 and
HT-1080 and the normal cell lines WI38 and Mefs.

| | Cancer Cell Line (μM) | | Normal Cell Line (μM) | |
|---|---|---|---|---|
| Analogs | NCI-H522 | HT1080 | WI38 | Mefs |
| RSL3 | 1.1 ± 0.89 | 0.14 ± 0.1 | 0.05 ± 0.01 | 0.04 ± 0.02 |
| CETZOLE (1) | 2.5 ± 0.32 | 3.07 ± 1.3 | 2.51 ± 0.04 | 2.41 ± 0.98 |
| 20 a | 0.17 ± 0.08 | 0.14 ± 0.06 | 1.53 ± 0.36 | 0.12 ± 0.06 |
| 20 b | 0.12 ± 0.06 | 0.15 ± 0.02 | 2.52 ± 0.92 | 0.28 ± 0.04 |
| 20 c | 0.13 ± 0.09 | 0.19 ± 0.09 | 1.19 ± 0.30 | 0.79 ± 0.04 |
| 23 | 1.36 ± 0.11 | 0.81 ± 0.14 | 2.51 ± 0.81 | 1.09 ± 0.46 |

To determine the selectivity of CETZOLE analogs for cancer over normal cells, cancer cell lines NCI-H522 and HT-1080 and the normal cell lines W138 (lung tissue fibroblast) and MEFs (Mouse Embryonic Fibroblasts) were treated with CETZOLE analogues using CETZOLE 1 and RSL3 as the reference compounds. The IC₅₀ values are shown in Table 2. Overall, WI38 cells were the most tolerant to the treatment, whereas MEF cells were partially tolerant and relatively less sensitive than both NCI-H522 and HT-1080 cells. To determine cytotoxicity at a therapeutically more relevant concentration, cells were treated at a single concentration of 1 M (FIG. 12). CETZOLE 1 and analogue 23 did not induce cell death at this concentration since they have a higher IC₅₀ value. RSL3 killed all cell lines indiscriminately. The analogs 20a, 20b, and 20c were highly selective and killed only the cancer cell lines NCI-H522 and HT-1080 at 1 M concentration, which indicates that these analogs are more selective for cancer cells over normal cells.

Analogues 20a and 23, and CETZOLE 1 were evaluated for antiproliferative activity in 60 human cancer cell line assay at the National Cancer Institute Developmental Therapeutics Program. The cell lines used in this assay belonged to leukemia, non-small-cell lung cancer, colon cancer, CNS cancer, melanoma, ovarian cancer, renal cancer, prostate cancer, and breast cancer. The compounds were first tested at a single dose of 10 μM. Analogue 20a was the most cytotoxic with a mean percentage growth of 1.24 (FIG. 13A). The renal cancer cell lines UO-31 and 786-0 and the ovarian cancer cell line IGROV1 showed a −95% percentage growth on 20a treatment, indicating its strong cytotoxic effect on these cell lines. For 23, the mean percentage growth was relatively higher at 27.24, but better than CETZOLE 1, which was the least cytotoxic. Analogs 20a and 23 were further tested in the NCI dose response assay (FIG. 1311). The leukemia, ovarian cancer, and renal cancer cell lines were found to be particularly sensitive to 20a. These results confirm that the CETZOLE analogs are more selective for cancer cells over normal cells yet cytotoxic to a wide range of cancer cell types.

### CETZOLE Probes and Protein Target Identification

Having established the SAR requirements of CETZOLE 1, a set of molecular probes was designed and synthesized to identify the CETZOLE protein target(s) (Scheme 5, FIGS. 14A-14D). They include the covalently binding propargyl probes, covalent binding fluorescence probes, photo-activatable affinity probes, and biotinylated affinity probe. The corresponding negative controls were synthesized in parallel. The pharmacophore layout of these probes was derived from the SAR of the CETZOLE templates analogues described above. Based on SAR information, the allylic and homo-allylic positions were chosen exclusively as points of modification to design the probes. Two approaches were adopted: allylic amidation and homo-allylic oxime O-alkylation (FIG. 14A). Reductive amination of the bromo-ketone 12 using methanolic ammonia and sodium borohydride in the presence of a titanium (iv) isopropoxide catalyst afforded the allyl bromo-amine 24. It was subjected to amide coupling with either coumarin carboxylic acid (25a), or biotin carboxylic acid (25b) using EDC as the coupling agent to obtain the amides NPa and NPb, respectively. Compounds NPa and NPb served as negative controls in biological assays. Sonogashira coupling with trimethylsilylacetylene, followed by desilylation with potassium carbonate in methanol, afforded the two probes: fluorescent coumarin probe PPa and the biotinylated probe PPb. The homoallylic CETZOLE oxime ether probes were synthesized in parallel with their corresponding negative controls following the procedure in FIG. 14A. The ketones 6 and 12 were converted to keto oximes 14 and 26, respectively, as described earlier. Both 14 and 26 were subjected to O-alkylation with the coumarin 28c (FIG. 14B) to obtain PPc and the corresponding negative probe NPc, respectively. Sodium borohydride reduction of oximes 14 and 26 afforded the oxime alcohols 19 and 27, respectively. Both were alkylated with bromo-alkanes 28d-28f in the presence of cesium carbonate to obtain probes PPd and PPe, respectively, and their corresponding negative control probes NPd and NPe, respectively. PPf and NPf purifications were not successful. Except for the commercially available propargyl bromide 28d, all other alkylating agents were synthesized as shown in FIGS. 141B-14D. The coumarin intermediate 28c was obtained by dropwise addition of bromoacetyl bromide 30 to a solution of coumarin amine 29 and TEA in DCM. The reaction yields were modest with good starting material recovery.

The benzophenone bromo-alkane 28e was obtained by selective EDC coupling of diaminobenzophenone 31 with 4-pentynoic acid 32 to obtain amide 33, followed by dropwise addition of bromoacetyl bromide 30 to a solution of 33 and TEA in DCM. To obtain intermediate 28f, Boc-protection of methyl 2-amino-5-bromobenzoate 34 to get 35, followed by installing an azide group at the para position employing Markiewicz and Helquist conditions, gave the azide 36. This was subjected to saponification followed by EDC coupling with propargyl amine 38 to obtain 39. Boc deprotection to get 40 was followed by acylation of the resulting amine with bromoacetyl bromide 30 afforded the bromo alkane 28f. The structures of the diverse library of probes synthesized are shown in FIGS. 14A-14D. They were screened for activity on NCI-H522, HT-1080, and MDA-MB-231 cells prior to using in target identification.

TABLE 3
Library of CETZOLE probes (PPa-PPf) and their corresponding negative controls
and (NPa-PPf) synthesized, and their IC₅₀ values on NCI-H522, HT-1080, and MDA-MB-231 cell lines.

| | | Cancer Cell line | | |
|---|---|---|---|---|
| | | | | MDA- |
| | | | | MB- |
| Ana- | | NCI-H522 | HT-1080 | 231 |
| logs | Structure | (μM) | (μM) | (μM) |
| 1 | 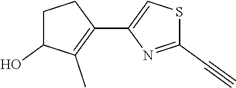 | 2.50 ± 0.32 | 0.96 ± 0.11 | 3.2 ± 0.2 |
| 13 | 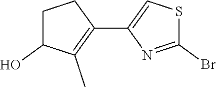 | >20 | >20 | >20 |
| PPc | 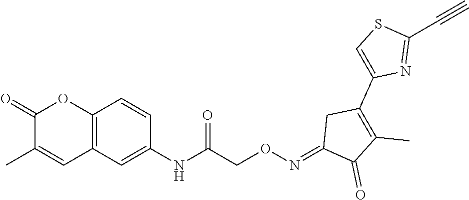 [SMILES: C#Cc1nc(C2=C(C)C(=O)/C(=N/OCC(=O)Nc3ccc4oc(=O)c(C)cc4c3)C2)cs1] | >20 | >20 | >20 |
| NPc | 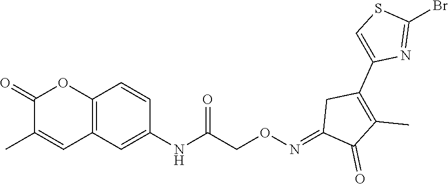 [SMILES: CC1=C(c2csc(Br)n2)C/C(=N\OCC(=O)Nc2ccc3oc(=O)c(C)cc3c2)C1=O] | >20 | >20 | >20 |
| PPa | 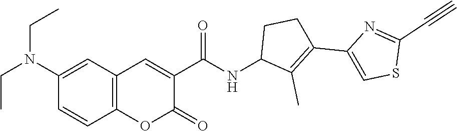 [SMILES: C#Cc1nc(C2=C(C)C(NC(=O)c3cc4cc(N(CC)CC)ccc4oc3=O)CC2)cs1] | 4.56 ± 1.43 | 4.58 ± 0.87 | 1.2 ± 1.03 |
| NPa | 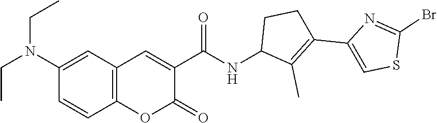 [SMILES: CCN(CC)c1ccc2oc(=O)c(C(=O)NC3CCC(c4csc(Br)n4)=C3C)cc2c1] | >20 | >20 | >20 |
| PPb | 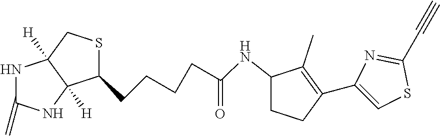 [SMILES: C#Cc1nc(C2=C(C)C(NC(=O)CCCC[C@@H]3SC[C@@H]4NC(=C)N[C@@H]43)CC2)cs1] | 17.90 ± 3.98 | 7.91 ± 1.9 | >20 |
| NPb | 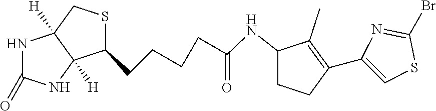 [SMILES: CC1=C(c2csc(Br)n2)CCC1NC(=O)CCCC[C@@H]1SC[C@@H]2NC(=O)N[C@@H]21] | >20 | >20 | >20 |
| PPd | 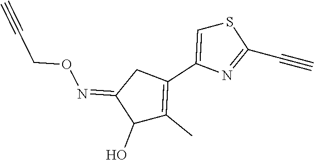 [SMILES: C#CCO/N=C1\CC(c2csc(C#C)n2)=C(C)C1O] | 2.31 ± 1.082 | 1.17 ± 0.12 | 2.7 ± 0.72 |
| NPd | 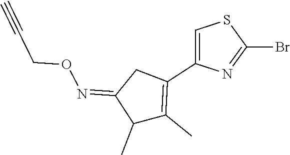 [SMILES: C#CCO/N=C1\CC(c2csc(Br)n2)=C(C)C1C] | >20 | >20 | >20 |
| PPe | 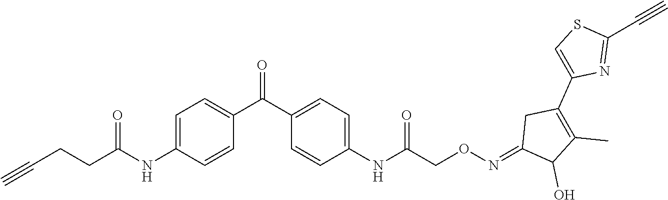 [SMILES: C#CCCC(=O)Nc1ccc(C(=O)c2ccc(NC(=O)CO/N=C3\CC(c4csc(C#C)n4)=C(C)C3O)cc2)cc1] | 2.60 ± 0.11 | 1.2 ± 0.01 | 1.33 ± 0.02 |
| NPe | 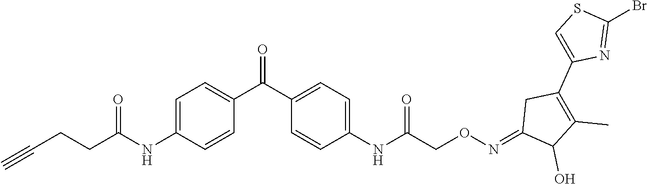 [SMILES: C#CCCC(=O)Nc1ccc(C(=O)c2ccc(NC(=O)CO/N=C3\CC(c4csc(Br)n4)=C(C)C3O)cc2)cc1] | >20 | >20 | >20 |
| PPf | 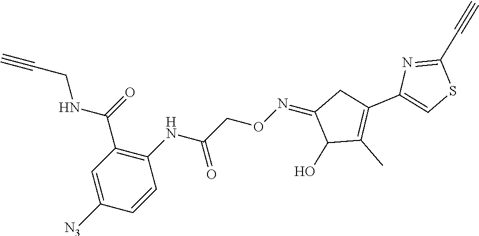 [SMILES: C#CCNC(=O)c1cc(N=[N+]=[N-])ccc1NC(=O)CO/N=C1/CC(c2csc(C#C)n2)=C(C)C1O] | ND | ND | ND |
| NPf | 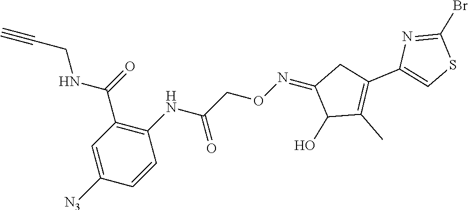 [SMILES: C#CCNC(=O)c1cc(N=[N+]=[N-])ccc1NC(=O)CO/N=C1/CC(c2csc(Br)n2)=C(C)C1O] | ND | ND | ND |

The structures of the CETZOLE probes PPa-PPf and their corresponding negative controls NPa-NPf are also depicted in FIG. 15.

The negative control probes did not induce significant cell death at the 20 μM maximum concentration tested. Probes PPd and PPe with oxime functions at the homoallylic position were the most cytotoxic with IC₅₀ values of 2.31±1.082 μM and 2.60±0.11 μM, respectively, against NCJ-H522 cell line (Table 3). A similar cytotoxicity trend was observed on HT-1080 and MDA-MB-231 cancer cell lines.

The allylic amide probe PPa designed as a covalent binder with a coumarin for in-gel visualization showed moderate cytotoxicity (IC₅₀ of 4.560±1.43 M). The allylic amide PPb with a biotin moiety, designed as an affinity probe for target protein pulldown, was five-fold less active (IC₅₀ 17.90±3.98 on NCI-H522 cell line) than CETZOLE 1. PPc did not induce any cytotoxicity at 20 μM maximum concentration as it had a ketone group at the allylic position instead of an alcohol group. Probes PPb and PPc were the least potent of all the molecules, and are not suitable as CETZOLE probes for protein target analysis. As shown in FIG. 16, NCI-H522 cell viability improved significantly on liproxstatin-1 co-treatment as compared to its absence. In this experiment, PPd gave the highest rescue of 75% viability, whilst PPe showed a significant cell viability at 45%. Overall, CETZOLE probes do induce radical-mediated cell death and are therefore indeed ferroptotic agents. The most active probes PPa, PPd, and PPe were used in further biological studies.

### Cellular Localization of CETZOLE Probes

The thiazole alkyne pharmacophore undergoes thiol addition. Therefore, PPd is believed to interact covalently with the target protein, and the propargyl alkyne serves as a clickable bio-orthogonal handle for attaching reporter tags. On the other hand, PPe has a photoactivatable benzophenone moiety that covalently binds to the target protein(s) upon photo irradiation. Bio-imaging experiments were conducted to determine the spatial localization of CETZOLE probes in HeLa cells using fluorescent reporter tags. For this, HeLa cells were incubated with probe PPd and the negative control NPd (FIG. 17A) at 37° C. for one-hour. The cells were washed with cold PBS, fixed with formaldehyde (5%), permeabilized with triton-X, and subjected to bioorthogonal click reaction with TAMRA-PEG-3-azide in the presence of tris-hydroxypropyltriazolylmethylanine (THPTA), copper (II) sulfate (CuSO₄), and sodium ascorbate. After multiple washings, cells were transferred on to a microscope viewing slide, stained with Hoechst dye, and observed under Olympus microscope (FIGS. 17C-17E). The images show intense intracellular aggregation of probe in the nucleus and vacuoles of cells treated with probe PPd compared to cells treated with the negative control NPd and DMSO.

The probe PPd was used in subsequent target protein identification studies since it proved to be the best in terms of potency and low fluorescence background noise in gels compared to others. Three cell lines sensitive to CETZOLE 1 (NCI-H522, HT-1080, and MDA-MB231) were tested. MDA-MB231 gave a cleaner gel than the others cell line (FIG. 18B). In a dose-dependent competitive binding assay, PPd was competed out by increasing concentrations of CETZOLE 1 (FIG. 18C). Three protein bands between 25 kD to 38 kD were completely competed out at 40 M CETZOLE 1 concentration and these protein bands are possible targets of CETZOLE 1.

Further competitive binding studies were conducted against known ferroptosis inducers CETZOLE 1, RSL3, erastin, ML210, ML160, and sulfasalazine (SSZ) at 20 μM concentration. Similar to CETZOLE 1, RSL3, ML210, and ML160 were able to compete out the same protein bands between 25 kD to 38 kD highlighted by the red star (FIG. 18D). Erastin and sulfasalazine treatment, on the other hand, did not show any significant competition for this band. This indicates that one possible protein target is GPX4 since only type II inhibitors competed out the same protein band.

### Mass Spectrometry

The selected cancer cell line (MDA-MB231) was treated with the probe 10 μM of PPd and incubated for 1 hour. The cells were lysed with lysis buffer, normalized, and subjected to bioorthogonal ligation with TAMRA-BIOTIN-PEG-3-AZIDE. The click chemistry reaction was carried as stated before. On completion, protein purification through precipitation and resuspension was done before streptavidin magnetic beads were added to the protein lysate and incubated overnight at 4° C. Thereafter, beads were subjected to multiple washings with the lysis buffer. The beads were split into two parts. 40 μL of lysis buffer and 10 μL of laemmle buffer were added to first aliquot which was separated on SDS PAGE gel to confirm successful protein pulldown (FIG. 19B). This experiment was conducted simultaneously with the negative control NPd and DMSO. The second aliquot was resolved on a 2 cm SDS-PAGE gel following standard LC-MS/MS sample preparation protocol before analyses by mass spectroscopy for qualitative protein target identification. The resulting data was compared against the Mascot data base to identify the enriched proteins. Spectral Abundance Factor (SAF) and subsequent Normalized Spectral Abundance Factor (NSAF) were calculated to determine the relative abundance of the enriched proteins unique to the positive control. The data is shown in FIGS. 19C-19D and is presented in Venn diagrams. The Venn diagram in FIG. 19C shows a total of 489 proteins identified to be enriched comparative to the negative control in this qualitative protein target evaluation. Cystathionine β-synthase (CBS), peroxiredoxins (PRDX4), ADP/ATP Carriers (ADT2), and glucose dehydrogenase (G6DP) are enriched proteins. These proteins are known to be associated with anti-oxidant production pathways and, therefore, linked to ferroptotic death. Cystathionine β-synthase, peroxiredoxins, and glucose dehydrogenase are examples of potential ferroptosis targets. CBS is a sulfide producing enzyme that catalyzes the conversion of L-homocysteine into cystathionine (an intermediate of L-cysteine) during transsulfuration. CBS inhibition leads to ferroptotic cell death. Peroxiredoxins, like other peroxidases, are involved in cytoprotective reduction of lipid peroxides. Peroxiredoxins are characterized by two sulfide residues on each unit which in the presence of ROS are readily oxidized to disulfides, preventing cellular oxidative damage and ferroptosis cell death. Peroxiredoxins inhibition leads to ROS accumulation and eventual ferroptosis cell death. Glucose dehydrogenase plays a key role in NADPH production through the pentose phosphate pathway. Over expression of glucose dehydrogenase is associated with poor prognosis of liver cancer and resistance to HRAS lethal molecules. GPX4 was validated as a target of CETZOLEs as well by western blotting using antibody, even though it was not enriched.

A follow up mass spectrometry analysis showed a different set of enriched proteins compared to previous analysis, yet these proteins are interesting as well since they have known association to anti-ferroptosis or anti-inflammatory functions. These enriched proteins are VDAC2, NEDD4L, PRDX4, GST01, TXN, and GLRX3 (FIGS. 20A-20B). Western blotting was used to validate the above proteins using anti-polyclonal antibodies for each protein of interest. The proteins of interest showed significant enrichment compared to the corresponding negative control. As an E3 ubiquitin ligase, NEDD4L targets ion channels, transporters, and is associated with cancer cell proliferation, and it was validated by western blotting (FIG. 20B, i). Similarly, glutathione S-transferase omega-1 GST01 proved to be a valid target by western blotting (FIG. 20B, ii). The rest of the enriched proteins validation was not successful yet they are still considered valid targets since the anti-bodies were the limitation.

### NCI-60 Cell Line Assay

FIG. 21 shows the results of a NCI-60 cell line assay at 10 nM with CETZOLE (1) and compounds 19, 20a, 23, and 18.

### CONCLUSION

A SAR study of CETZOLE 1 yielded highly potent ferroptotic agents in comparison to CETZOLE 1. The most potent analogs 20a-20c act as prodrugs that release the active agent upon hydrolysis by esterases. Rescue and flow cytometry assays confirmed these analogs to be classic ferroptosis agents that induce cell death through ROS accumulation. These agents are more cytotoxic to cancer cells over normal cells. Target protein identification studies using molecular probes led to the identification of cystathionine β-synthase, peroxiredoxins, ADP/ATP Carriers (SLC25A5), glucose dehydrogenase, VDAC, NEDD4L, GST01, and thioredoxin as possible targets of CETZOLEs. These proteins and GPX4 were verified through western blotting and are known to be associated with cellular antioxidant pathways.

### Experimental Procedure

### Cell Lines and Culture Conditions

Cell lines were cultured in a humidified atmosphere containing 10% CO₂ in Dulbecco's modified Eagle's medium (Mediatech, Inc) supplemented with 10% fetal bovine serum (Atlanta Biologicals). Cell types used MEFs, HT1080 (human fibrosarcoma cells), NARF2 (osteosarcoma), WI-38 (human embryonic lung fibroblast), MDA MB 231 (breast cancer), HCT-116 (human colon), and NCI-H522 (Non-Small Cell Lung Cancer) cell lines. To determine viability throughout these examples, 5,000 cells were plated per well of a 96-well plate and drugs were added 1 day later. Cells were stained 1 to 3 days later with a saturated solution of methylene blue in 50% ethanol. Plates were rinsed and retained dye was quantified by spectrophotometry. Absorbance was normalized to DMSO and given as 1 or 100% for cell viability. Results are representative of at least two independent experiments. Statistical significance was assessed using the Student's t test. All commercially available chemicals were obtained from Cayman Chemicals unless otherwise noted. Compound CETZOLE 1 and the analogs were synthesized as highlighted.

### Fluorescence Gel and Western Blotting

Cells were harvested by scraping and lysed in a buffer solution containing: 50 mM Tris (pH 7.4), 150 mM NaCl, 0.5% NP-40, 1 μg/ml aprotinin, 2 μg/ml leupeptin, 1 μg/ml pepstatin A, 1 mM DTT, 1 mM PMSF. 5 mM sodium fluoride, and 2 mM sodium vanadate for 20 min on ice. Insoluble debris was removed by centrifugation at 16,000 g for 20 min at 4° C. Equal amounts of protein for each sample (determined using BCA protein assay kit-Pierce) were separated by SDSPAGE after click reaction. Gels were transferred to polyvinylidene difluoride membranes (Millipore), blocked in a solution containing 5% (w/v) nonfat dry milk dissolved in PBS containing 0.05% (v/v) Tween 20, and probed with antibodies as indicated. For phospho-specific antibodies, membranes were blocked in 5% (w/v) bovine serum albumin in Tween 20 containing Tris buffered saline. Antibodies were generally diluted in the blocking solution at 1:1000. Primary antibodies recognizing GPX4, peroxiredoxin-4, CBS, NEDD4L, and GST01 (Abelonal #A1933, #A1486, #A1427, #A8085, #A4636) were generally incubated at 4° C. overnight (or 1.5 h at room temperature). Signals were detected using horseradish peroxidase-conjugated secondary antibodies (Bio-Rad) and enhanced chemiluminescence (Bio-Rad). Western blot images were mostly taken by a ChemiDoc, and the digital images were analyzed using ImageJ software.

### Lipid ROS Measurement

NCI-H522 cells were seeded at 1.5×10⁵ cells per 9-cm plate. The next media was replaced with fresh media before treated with drugs along with BODIPY581/591-C11 dye (0.5 μM) (Thermo Fisher). Forty-eight washed with 1×PBS, and resuspended in 2% FBS containing 1×PBS. Cells were analyzed in FITC channel using a BD LSR Fortessa FAC Scanner. Twenty thousand events per condition were analyzed from three independent samples. The experiments were performed twice, each time with triplicate samples (n=6). Collected data were processed with FlowJo v10 software. Lipid ROS measurement in TR9-7 or NARF2 cells was performed similarly at the indicated conditions as mentioned in the respective figure legends. Where appropriate, dead cells were excluded by gating. When gates were applied, they were identical across samples within an experiment.

### Chemistry

### Materials and Methods

All chemicals and solvents were purchased from commercial sources and used without further purification, unless stated otherwise. Anhydrous tetrahydrofuran was freshly distilled from sodium and benzophenone before use. ¹H and ¹³C NMR spectra were recorded on Brucker Avance 600 MHz, INOVA 600 MHz and Varian VXRS 400 MHz NMR spectrometers in deuterated solvents using residual un-deuterated solvents as internal standard. High-resolution mass spectra (HRMS) were recorded on a Waters Synapt high-definition mass spectrometer (HDMS) equipped with nano-ESI source. Melting points were determined using a Fisher-Johns melting point apparatus. Purifications of crude products were performed by either flash chromatography on silica gel (40-63 p) from Sorbent Technologies or on a Teledyne ISCO CombiFlash Companion chromatography system on RediSep prepacked silica cartridges. Thin layer chromatography (TLC) plates (20 cm×20 cm) were purchased from Sorbent Technologies (catalog #4115126) and were viewed under Model UVG-54 mineral light lamp UV-254 nm. A Shimadzu Prominence HPLC with an LCT20AT solvent delivery system coupled to a Shimadzu Prominence SPD 20AV Dual wavelength UV/Vis absorbance Detector, a Shimadzu C18 column (1.9 m, 2.1 mm×50 in), and HPLC grade solvents (MeOH, H₂O with 0.1% formic acid) were used to determine the purity of compounds by HPLC.

### 1-(2-Bromothiazol-4-yl)hexane-1,4-dione (11)

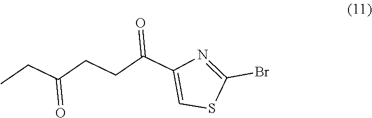

2-Bromothiazole-4-carbaldehyde 8 (5 g, 26.04 mmol, 1 equiv.) and 3-ethyl-5-(2-hydroxyethyl)-4-methyl)thiazolium 10 (0.654 g, 2.6 mmol, 0.1 equiv.) were dissolved in anhydrous tetrahydrofuran (20 mL) in an oven-dried round bottom flask equipped with a magnetic stir bar. Pent-1-en-3-one 9 (2.19 g, 2.71 mL, 26.01 mmol, 1 equiv.) and triethylamine (2.63 g, 3.63 mL, 26.04 mmol, 1.1 equiv.) were added, and the reaction mixture was heated under reflux overnight whilst monitored by TLC. On completion, the reaction mixture was quenched with aqueous ammonium chloride (10 mL) and extracted with ethyl acetate (3×20 mL). The combined organic phase was dried over anhydrous sodium sulfate, filtered, and concentrated under reduced pressure. The crude product was purified by flash chromatography on silica gel in 0 to 70% ethyl acetate in hexanes to obtain 1-(2-bromothiazol-4-yl)hexane-1,4-dione 11 (23 mmol, 83%) as a white solid. ¹H NMR (600 MHz, CDCl₃) δ 8.08 (s, 1H), 3.36 (d, J=4.5 Hz, 2H), 2.86 (d, J=5.3 Hz, 2H), 2.55 (d, J=7.2 Hz, 2H), 1.09 (d, J=7.2 Hz, 3H). ¹³C NMR (151 MHz, CDCl₃) δ 209.8, 192.7, 154.1, 136.1, 128.6, 35.9, 35.7, 34.0, 7.9.

### 3-(2-Bromothiazol-4-yl)-2-methylcyclopent-2-en-1-one (12)

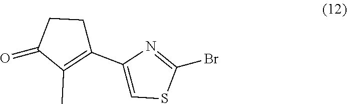

1-(2-Bromothiazol-4-yl)hexane-1,4-dione (11) (6 g, 21.73 mmol, 1.0 equiv.) was dissolved in anhydrous ethanol (50 mL) in an oven-dried round bottom flask equipped with a magnetic stir bar. Sodium hydroxide (2.48 g, 86.91 mmol, 4 equiv.) pellets were gradually added and the reaction mixture was stirred at room temperature whilst monitored by TLC. On completion, the reaction mixture was quenched with aqueous ammonium chloride (40 mL). The ethanol was removed under reduced pressure and the residue extracted with ethyl acetate (3×30 mL). The combined organic phase was dried over anhydrous sodium sulfate, filtered, and concentrated under reduced pressure. The crude product was purified by flash chromatography on silica gel in 0 to 50% ethyl acetate in hexanes to obtain 3-(2-bromothiazol-4-yl)-2-methylcyclopent-2-en-1-one 12 (21.31 mmol, 98%) as a brownish cream solid. ¹H NMR (600 MHz, CDCl₃) δ 7.58 (s, 1H), 2.99-2.95 (m, 2H), 2.58-2.55 (m, 2H), 2.14 (s, 3H). ¹³C NMR (151 MHz, CDCl₃) δ 209.7, 156.7, 152.7, 137.6, 136.2, 123.4, 123.3, 33.7, 27.8, 10.1. FIG. 22A shows the ¹H NMR spectrum of 1-(2-bromothiazol-4-yl)hexane-1,4-dione (12), and FIG. 22B shows the ¹³C NMR spectrum of 1-(2-bromothiazol-4-yl)hexane-1,4-dione (12).

### General Procedure for Sonogashira Coupling Reaction

Bromo-ketone (1 equiv.), bis(triphenylphosphine)palladium(II)dichloride (2.5 mol %), copper(I) iodide (2.5 mol %), and triphenylphosphine (5 mol %) were dissolved in anhydrous dichloroethane (1 mL) under nitrogen in an oven-dried round bottom flask equipped with a magnetic stir bar. Triethylamine (1.1 equiv.) and ethynyltrimethylsilane (1.1 equiv.) were added, and the reaction mixture was heated under reflux overnight whilst monitored by TLC. On completion, dichloroethane was removed under reduced pressure before diluting the mixture with brine (5 mL) and extracting with ethyl acetate (3×5 mL). The combined organic phase was dried over anhydrous sodium sulfate, filtered, and concentrated under reduced pressure. The crude product was purified by flash column chromatography on silica gel in 0 to 40% ethyl acetate in hexanes to obtain a pure product of interest.

### 2-Methyl-3-(2-((trimethylsilyl)ethynyl)thiazol-4-yl)cyclopent-2-en-1-one (6)

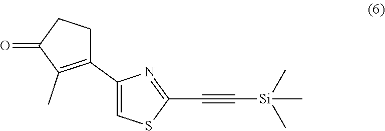 [SMILES: CC1=C(c2csc(C#C[Si](C)(C)C)n2)CCC1=O]

Synthesized according to the general Sonogashira coupling reaction procedure using 3-(2-bromothiazol-4-yl)-2-methylcyclopent-2-en-1-one (12) and ethynyltrimethylsilane. (5.45 mmol, 70.29%). Brown solid. ¹H NMR (600 MHz, CDCl₃) δ 7.63 (s, 1H), 3.06-2.99 (m, 2H), 2.59-2.54 (m, 2H), 2.15 (dt, J=4.1, 2.0 Hz, 3H), 0.33-0.28 (m, 9H), as shown in FIG. 23A. ¹³C NMR (151 MHz, CDCl₃) δ 209.9, 157.8, 152.7, 148.3, 137.5, 121.6, 102.1, 96.1, 33.7, 28.2, 10.2, −0.5, as shown in FIG. 23B.

### General Procedure for Trimethylsilyl Deprotection Reaction

The trimethylsilyl ketone (1.0 equiv.) was dissolved in anhydrous methanol (1.0 mL) in an oven-dried round bottom flask equipped with a magnetic stir bar. Potassium carbonate (0.5 equiv.) was added, and the reaction mixture was stirred at room temperature whilst monitored by TLC. On completion, the reaction mixture was quenched with aqueous ammonium chloride (4 mL) and extracted with ethyl acetate (3×3 mL). The combined organic phase was dried over anhydrous sodium sulfate, filtered, and concentrated under reduced pressure. The crude product was purified by flash chromatography on silica gel in 0 to 50% ethyl acetate in hexanes to obtain the pure product of interest.

### 3-(2-Ethynylthiazol-4-yl)-2-methylcyclopent-2-en-1-one (7)

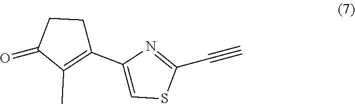

Synthesized according to the general trimethylsilyl deprotection reaction procedure. (0.64 mmol, 88%). Light brown solid. ¹H NMR (600 MHz, CDCl₃) δ 7.67 (s, 1H), 3.56 (s, 1H), 3.05-3.00 (m, 2H), 2.59-2.55 (m, 2H), 2.16 (s, 3H), as shown in FIG. 24A. ¹³C NMR (151 MHz, CDCl₃) δ 209.8, 157.4, 152.9, 147.3, 137.7, 121.7, 83.0, 33.7, 28.1, 10.2, as shown in FIG. 24B.

### General Procedure for Reduction of Ketone

The ketone (1.0 equiv.) was dissolved in anhydrous methanol (5 mL) at 0° C. in an oven-dried round bottom flask equipped with a magnetic stir bar. Sodium borohydride (2 equiv.) was added in portions. The reaction mixture was allowed to warm to room temperature and monitored by TLC. On completion, the reaction mixture was quenched with aqueous ammonium chloride (10 mL) and extracted with ethyl acetate (3×5 mL). The combined organic phase was dried over anhydrous sodium sulfate, filtered, and concentrated under reduced pressure. The crude product was purified by flash chromatography on silica gel in 0 to 70% ethyl acetate in hexanes to obtain the pure alcohol.

### 3-(2-Ethynylthiazol-4-yl)-2-methylcyclopent-2-en-1-ol (1)

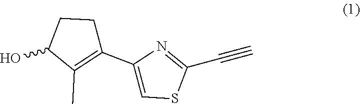

Synthesized according to the general procedure for reduction of ketone. (0.433 mmol, 88%). Brown semi liquid; mp 230° C. ¹H NMR (600 MHz, CDCl₃) δ 7.12 (s, 1H), 4.72 (s, 1H), 3.46-3.42 (m, 1H), 2.88-2.79 (m, 1H), 2.67-2.58 (m, 1H), 2.38 (tdd, J=12.9, 8.5, 4.1 Hz, 1H), 2.17-2.06 (m, 3H), 1.79-1.68 (m, 1H), as shown in FIG. 25A. ¹³C NMR (151 MHz, CDCl₃) δ 153.9, 146.4, 140.1, 130.7, 117.3, 82.1, 81.9, 32.72, 32.5, 13.3, as shown in FIG. 25B.

### 3-(2-Bromothiazol-4-yl)-2-methylcyclopent-2-en-1-ol (13)

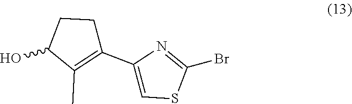

Synthesized according to the general procedure for reduction of ketone. (0.433 mmol, 88%). Brown semi liquid. ¹H NMR (600 MHz, CDCl₃) δ 7.08-7.05 (m, 1H), 4.77 (t, J=6.1 Hz, 1H), 2.84 (dddt, J=15.1, 9.0, 3.9, 1.9 Hz, 1H), 2.66-2.58 (m, 1H), 2.46-2.38 (m, 1H), 2.17 (dd, J=2.8, 1.9 Hz, 3H), 1.78 (dddd, J=10.6, 9.0, 5.0, 3.4 Hz, 1H), as shown in FIG. 26A. ¹³C NMR (151 MHz, CDCl₃) δ 140.1, 134.9, 133.7, 130.1, 118.7, 116.6, 82.1, 39.5, 32.4, 32.4, 13.2, as shown in FIG. 26B.

### General Procedure for Oxime Formation α-to Ketone Function

The ketone (1.0 equiv.) was dissolved in anhydrous methanol (1.0 mL) in an oven-dried round bottom flask equipped with a magnetic stir bar. At 0° C., 5N hydrochloric acid (0.1 equiv.) and tert-butyl nitrite (2 equiv.) were added slowly and the reaction mixture was allowed to warm to room temperature. The product precipitated was collected by filtration, washed with water (5 mL), and dried to obtain the pure product.

### (E)-3-(2-Ethynylthiazol-4-yl)-5-(hydroxyimino)-2-methylcyclopent-2-en-1-one (14)

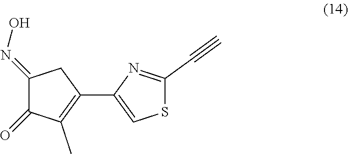 [SMILES: C#Cc1nc(C2=C(C)C(=O)/C(=N/O)C2)cs1]

Synthesized according to the general procedure for oxime formation α-to ketone function. (1.2 mmol, 99%). White solid; mp 140° C. ¹H NMR (600 MHz, CD₃OD) δ 8.24 (s, 1H), 4.33 (d, J=1.2 Hz, 1H), 3.71 (s, 2H), 2.25 (s, 3H), as shown in FIG. 27A. ¹³C NMR (151 MHz, CD₃OD) δ 192.4, 151.7, 150.1, 148.8, 123.6, 102.8, 95.8, 32.3, 10.7, −0.5, as shown in FIG. 27B. HRMS calculated for C₁₁H₈N₂O₂S (M+H) 233.2570 found 233.087.

### (E)-3-(2-Bromothiazol-4-yl)-5-(hydroxyimino)-2-methylcyclopent-2-en-1-one (26)

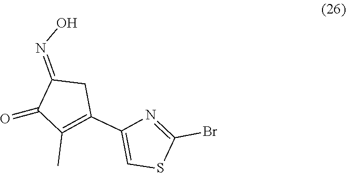

Synthesized according to the general keto-oxime reaction procedure. (1.25 mmol, 99%). White solid; 150° C. ¹H NMR (600 MHz, DMSO-d₆) δ 12.45 (s, 1H), 8.39 (s, 1H), 3.60-3.55 (m, 2H), 2.15-2.11 (m, 3H), as shown in FIG. 28A. ¹³C NMR (151 MHz, DMSO-d₆) δ 192.5, 152.6, 151.5, 149.2, 138.6, 137.5, 129.0, 55.4, 30.1, 10.6, as shown in FIG. 28B.

### (E)-4-(2-Ethynylthiazol-4-yl)-2-hydroxy-3-methylcyclopent-3-en-1-one oxime (19)

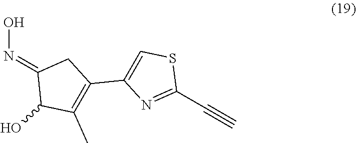 [SMILES: C#Cc1nc(C2=C(C)C(O)/C(=N/O)C2)cs1]

Synthesized according to the general ketone for reduction of ketone. (0.107 mmol, 79%). Brown solid; mp 130° C. ¹H NMR (600 MHz, acetone-d₆) δ 10.19 (s, 1H), 7.70 (s, 1H), 4.94 (s, 1H), 4.64 (d, J=7.1 Hz, 1H), 4.35 (s, 1H), 3.50 (ddd, J=77.9, 21.3, 2.1 Hz, 2H), 2.24 (s, 3H), as shown in FIG. 29A. ¹³C NMR (151 MHz, acetone-d₆) δ 160.4, 153.2, 146.5, 139.6, 126.5, 119.0, 83.4, 77.8, 76.4, 33.6, 12.8, as shown in FIG. 29B. HRMS calculated for C₁₁H₁₀N₂O₂S (M+H) 235.2730 found 235.0589.

### (E)-4-(2-Bromothiazol-4-yl)-2-hydroxy-3-methylcyclopent-3-en-1-one oxime (27)

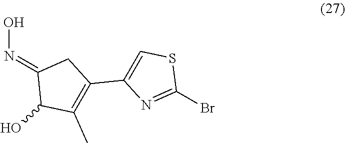

Synthesized according to the general ketone for reduction of ketone. (0.10 mmol, 80%). White solid; mp 151° C. ¹H NMR (600 MHz, CD₃OD) δ 7.53 (s, 1H), 4.64 (s, 1H), 3.57 (dd, J=21.4, 2.1 Hz, 1H), 3.46-3.39 (m, 1H), 2.21 (t, J=5.8 Hz, 3H), as shown in FIG. 30A. ¹³C NMR (151 MHz, CD₃OD) δ 160.6, 152.5, 138.8, 135.2, 126.7, 120.7, 77.7, 33.1, 12.0, as shown in FIG. 30B.

### General Procedure for Acylation Reaction

The alcohol-oxime (1.0 equiv.) was dissolved in anhydrous dichloromethane (1 mL) in an oven-dried round bottom flask equipped with a magnetic stir bar. At 0° C., triethylamine (3.0 equiv.) and corresponding acyl anhydride/acyl chloride (3.0 equiv.) were added and the mixture was allowed to warm up to room temperature whilst monitored by TLC. On completion, the reaction mixture was quenched with aqueous sodium bicarbonate (4 mL) and extracted with ethyl acetate (3×3 mL). The combined organic phase was dried over anhydrous sodium sulfate, filtered, and concentrated under reduced pressure. The crude product was purified by flash column chromatography on silica gel in 0 to 30% ethyl acetate in hexanes to obtain the pure acyl product.

### (E)-5-(Acetoxyimino)-3-(2-ethynylthiazol-4-yl)-2-methylcyclopent-2-en-1-yl acetate (20a)

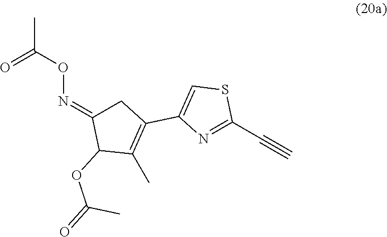 [SMILES: C#Cc1nc(C2=C(C)C(OC(C)=O)/C(=N/OC(C)=O)C2)cs1]

Synthesized according to the general procedure for acylation reaction. (0.031 mmol, 46%). Cream brown solid; mp 160° C. ¹H NMR (600 MHz, CDCl₃) δ 7.32 (s, 1H), 6.31 (s, 1H), 3.78 (ddd, J=106.3, 22.0, 1.5 Hz, 3H), 3.53 (d, J=0.8 Hz, 1H), 2.22 (d, J=0.6 Hz, 3H), 2.15 (d, J=2.4 Hz, 7H), as shown in FIG. 31A. ¹³C NMR (151 MHz, CDCl₃) δ 169.9, 168.5, 166.2, 151.5, 147.0, 135.1, 129.5, 119.2, 82.7, 78.7, 36.4, 20.9, 19.6, 13.5. HRMS calculated for C₁₅H₁₄N₂O₄S (M+H) 341.3470 found 341.0578, as shown in FIG. 31B.

### (E)-3-(2-Ethynylthiazol-4-yl)-2-methyl-5-((propionyloxy)imino)cyclopent-2-en-1-yl propionate (20b)

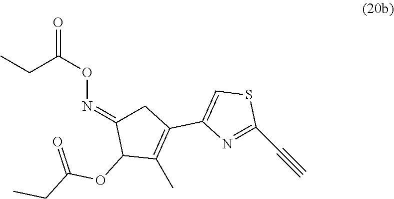 [SMILES: C#Cc1nc(C2=C(C)C(OC(=O)CC)/C(=N/OC(=O)CC)C2)cs1]

Synthesized according to the general procedure for acylation reaction. (0.012 mmol, 35%). A brown solid; mp 120° C. ¹H NMR (600 MHz, Acetone-d₆) δ 7.80 (d, J=5.0 Hz, 1H), 6.33 (s, 1H), 4.33 (d, J=5.2 Hz, 1H), 3.83-3.61 (m, 2H), 2.46 (dt, J=12.8, 4.9 Hz, 2H), 2.42-2.35 (m, 2H), 2.12 (s, 3H), 1.10 (tt, J=8.5, 6.3 Hz, 6H), as shown in FIG. 32A. ¹³C NMR (151 MHz, Acetone-d₆) δ 172.8, 170.9, 166.0, 151.8, 146.8, 134.8, 129.0, 120.5, 83.8, 78.1, 76.2, 35.5, 26.9, 25.5, 12.5, 8.6, 8.3, as shown in FIG. 32B. HRMS calculated for C₁H₁₈N₂O₄S (M+H) 347.4010 found 347.0912.

### (E)-5-((Butyryloxy)imino)-3-(2-ethynylthiazol-4-yl)-2-methylcyclopent-2-en-1-yl butyrate (20c)

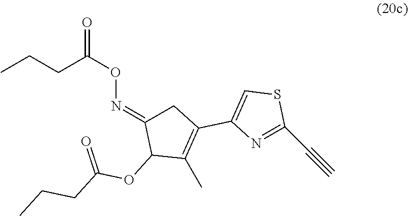 [SMILES: C#Cc1nc(C2=C(C)C(OC(=O)CCC)/C(=N/OC(=O)CCC)C2)cs1]

Synthesized according to the general procedure for acylation reaction. (0.016 mmol, 37%). Brown solid; mp 100° C. ¹H NMR (600 MHz, CDCl₃) δ 7.33 (s, 1H), 6.34 (s, 1H), 3.78 (dd, J=92.1, 2.3 Hz, 2H), 3.54 (s, 1H), 2.47 (t, J=7.4 Hz, 2H), 2.40 (td, J=7.3, 6.0 Hz, 2H), 2.20 (s, 3H), 1.74 (ddd, J=31.0, 14.8, 7.4 Hz, 4H), 1.08-0.95 (m, 6H), as shown in FIG. 33A. ¹³C NMR (151 MHz, CDCl₃) δ 172.8, 170.9, 166.1, 151.9, 146.8, 134.8, 129.2, 120.5, 83.8, 78.1, 76.2, 35.5, 26.9, 25.5, 12.5, 8.56, 8.3, as shown in FIG. 33B. HRMS calculated for C₁₉H₂₂N₂O₄S (M+H) 375.4550 found 375.1360.

### (E)-3-(2-Ethynylthiazol-4-yl)-2-methyl-5-((octanoyloxy)imino)cyclopent-2-en-1-yl octanoate (20e)

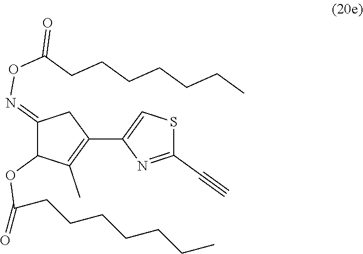

Synthesized according to the general procedure for acylation reaction. (0.034 mmol, 71%). Brown solid; mp 125° C. ¹H NMR (600 MHz, CDCl₃) δ 7.37 (d, J=1.9 Hz, 1H), 6.35 (d, J=0.8 Hz, 1H), 3.94-3.64 (m, 2H), 2.52-2.35 (m, 4H), 2.22-2.16 (m, 4H), 1.74-1.63 (m, 4H), 0.96-0.85 (m, 9H), as shown in FIG. 34A. ¹³C NMR (151 MHz, CDCl₃) δ 172.1, 170.1, 166.3, 133.5, 129.6, 118.6, 117.9, 78.2, 49.9, 35.6, 33.7, 32.2, 31.6, 27.1, 24.9, 24.6, 22.4, 13.4, 12.5, as shown in FIG. 34B. HRMS calculated for C₂₇H₃₈N₂O₄S (M+H) 487.6710 found 487.2350.

### General Procedure for the Conversion of Ketone to Oxime

The ketone (1.0 equiv.), O-benzylhydroxylamine hydrochloride (5 equiv.), and sodium acetate (7 equiv.) were dissolved in methanol/water (3:1) (3 mL) in a round bottom flask equipped with a magnetic stir bar. The reaction mixture was heated under reflux overnight whilst monitored by TLC. On completion the mixture was poured into ice water and the product precipitated was collected by filtration, and washed with ice water to obtain the pure oxime.

### (E)-3-(2-Ethynylthiazol-4-yl)-2-methylcyclopent-2-en-1-one O-benzyl oxime (23)

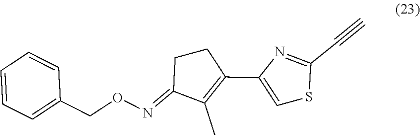 [SMILES: C#Cc1nc(C2=C(C)/C(=N/OCc3ccccc3)CC2)cs1]

Synthesized according to the general procedure for conversion of ketone to oxime (0.042 mmol, 24%). Brown semi-liquid. ¹H NMR (600 MHz, CDCl₃) δ 7.38 (d, J=7.5 Hz, 2H), 7.33 (t, J=7.5 Hz, 2H), 7.30-7.26 (m, 2H), 5.15 (s, 2H), 3.47 (s, 1H), 2.88-2.83 (m, 2H), 2.74 (dd, J=6.9, 4.2 Hz, 2H), 2.20 (s, 3H), as shown in FIG. 35A. ¹³C NMR (151 MHz, CDCl₃) δ 168.6, 153.5, 146.7, 142.2, 138.3, 134.6, 128.3, 128.1, 127.7, 118.6, 82.3, 76.1, 31.4, 24.8, 11.4, as shown in FIG. 35B. HRMS calculated for C₁₈H₁₆N₂OS (M+H) 309.3990 found 309.111.

### General Procedure for Alkylation of Alcohol-Oxime

The alcohol-oxime (1.0 equiv.) and caesium carbonate (2 equiv.) were dissolved in anhydrous acetonitrile (1.0 mL) in an oven-dried round bottom flask equipped with a magnetic stir bar. The corresponding alkyl bromide (1.5 equiv.) was added slowly and the reaction mixture stirred overnight and monitored by TLC. On completion, the reaction mixture was quenched with water (4 mL) and extracted with ethyl acetate (3×3 mL). The combined organic phase was dried over anhydrous sodium sulfate, filtered, and concentrated under reduced pressure. The crude product was purified by flash chromatography on silica gel in 0 to 50% ethyl acetate in hexanes to obtain the pure product of interest.

### (Z)-4-(2-Ethynylthiazol-4-yl)-2-hydroxy-3-methylcyclopent-3-en-1-one O-benzyl oxime (21)

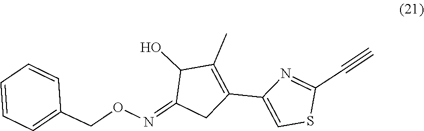 [SMILES: C#Cc1nc(C2=C(C)C(O)/C(=N\OCc3ccccc3)C2)cs1]

Synthesized according to the general procedure for alkylation of alcohol-oxime with benzyl bromide 15. (0.037 mmol, 46%). Clear oil. ¹H NMR (600 MHz, CDCl₃) δ 7.41-7.34 (m, 4H), 7.34-7.30 (m, 1H), 7.23 (s, 1H), 5.20 (s, 1H), 5.08 (s, 1H), 3.70 (dd, J=21.6, 2.1 Hz, 2H), 3.52-3.45 (m, 1H), 2.25 (s, 3H), as shown in FIG. 36A. ¹³C NMR (151 MHz, CDCl₃) δ 161.5, 152.7, 146.7, 138.4, 137.6, 128.4, 128.1, 127.9, 118.4, 82.3, 78.5, 76.3, 36.6, 34.9, 13.4, as shown in FIG. 36B. HRMS calculated for C₁₈H₁₆N₂O₂S (M+H) 324.3980 found 323.0866.

### 3-(2-Bromothiazol-4-yl)-2-methylcyclopent-2-en-1-amine (24)

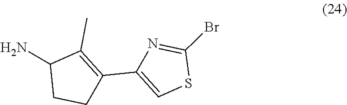

3-(2-Bromothiazol-4-yl)-2-methylcyclopent-2-en-1-one 12 (200 mg, 0.708 mmol, 1 equiv.) and titanium isoproproxide (218.50 mg, 0.768 mmol, 1 equiv.) were dissolved in anhydrous methanol (0.5 mL) in an oven-dried round bottom flask equipped with a magnetic stir bar. 7N aqueous ammonia solution (65.47 mg, 0.549 mL, 3.84 mmol, 5 equiv.) was added and the reaction was monitored by TLC. The mixture was cooled to 0° C. before the slow addition of sodium borohydride (58.17 mg, 1.54 mmol, 2 equiv.), and the reaction mixture was allowed to warm up to room temperature whilst monitored by TLC. On completion, the reaction was quenched with 5% hydrochloric acid solution and extracted with ethyl acetate (3×5 mL). The aqueous phase was basified with sodium hydroxide and extracted with ethyl acetate (3×5 mL). The combined organic phase was dried over anhydrous sodium sulfate, filtered, and concentrated under reduced pressure to yield pure 3-(2-bromothiazol-4-yl)-2-methylcyclopent-2-en-1-amine (24) (0.045 mmol, 40%) as a clear liquid. ¹H NMR (600 MHz, CD₃OD) δ 7.29 (s, 1H), 3.85 (t, J=6.9 Hz, 1H), 2.78-2.71 (m, 1H), 2.63-2.54 (m, 1H), 2.36-2.28 (m, 1H), 2.08 (s, 3H), 1.63-1.55 (m, 1H), as shown in FIG. 37A. ¹³C NMR (151 MHz, CD₃OD) δ 152.0, 135.4, 133.8, 132.7, 121.5, 61.6, 32.4, 27.1, 12.1, as shown in FIG. 37B.

### General Procedure for Amide Coupling Using EDC-HCl

The carboxylic acid (1 equiv.), EDC-HCl (1.5 equiv.), and 4-dimethylaminopyridine (10 mmol %) were dissolved in anhydrous dichloromethane (3 mL) in an oven-dried round bottom flask equipped with a magnetic stir bar. The amine (1.1 equiv.) was added and the reaction was monitored by TLC. On completion, water (5 mL) was added and extracted with ethyl acetate (3×5 mL). The combined organic phase was dried over anhydrous sodium sulfate, filtered, and concentrated under reduced pressure. The crude product was purified by flash chromatography on silica gel in 0 to 50% ethyl acetate in hexanes to obtain the pure amide.

### N-(3-(2-Bromothiazol-4-yl)-2-methylcyclopent-2-en-1-yl)-7-(diethylamino)-2-oxo-2H-chromene-3-carboxamide (NPa)

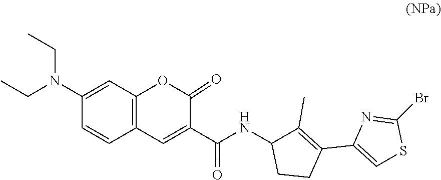 [SMILES: CCN(CC)c1ccc2cc(C(=O)NC3CCC(c4csc(Br)n4)=C3C)c(=O)oc2c1]

Synthesized according to the general procedure for amide coupling using EDC-HCl with 7-(diethylamino)-2-oxo-2H-chromene-3-carboxylic acid (25a). (0.159 mmol, 83%). Orange solid; mp 200° C. ¹H NMR (600 MHz, CDCl₃) δ 8.84 (d, J=8.8 Hz, 1H), 8.71 (s, 1H), 7.42 (d, J=9.0 Hz, 1H), 7.27 (s, 1H), 7.03 (s, 1H), 6.64 (dd, J=9.0, 2.4 Hz, 1H), 3.44 (q, J=7.1 Hz, 4H), 2.86-2.74 (m, 1H), 2.72-2.58 (m, 1H), 2.55-2.45 (m, 1H), 2.07 (d, J=24.4 Hz, 3H), 1.25-1.16 (m, 9H), as shown in FIG. 38A. ¹³C NMR (151 MHz, CDCl₃) δ 206.9, 162.9, 162.7, 157.6, 153.6, 152.5, 149.7, 148.2, 138.8, 134.8, 131.1, 130.1, 118.5, 110.2, 109.9, 109.6, 108.4, 96.7, 96.5, 60.2, 53.5, 52.3, 45.1, 45.1, 32.9, 30.9, 30.5, 13.4, 12.4, as shown in FIG. 38B.

### 7-(Diethylamino)-N-(3-(2-ethynylthiazol-4-yl)-2-methylcyclopent-2-en-1-yl)-2-oxo-2H-chromene-3-carboxamide (PPa)

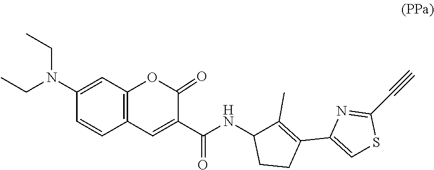 [SMILES: C#Cc1nc(C2=C(C)C(NC(=O)c3cc4ccc(N(CC)CC)cc4oc3=O)CC2)cs1]

Synthesized according to the general procedure for Sonogashira coupling reaction. (0.045 mmol, 70%). Orange solid; mp 210° C. ¹H NMR (600 MHz, CDCl₃) δ 8.88 (d, J=8.0 Hz, 1H), 8.75 (s, 1H), 7.45 (d, J=8.6 Hz, 1H), 7.16 (s, 1H), 6.66 (d, J=8.7 Hz, 1H), 6.51 (s, 1H), 5.30 (s, 1H), 3.50-3.43 (m, 3H), 2.89 (s, 1H), 2.76 (s, 1H), 2.53 (d, J=8.8 Hz, 1H), 2.13 (s, 2H), 1.80 (s, 1H), 1.33-1.17 (m, 8H), as shown in FIG. 39A. ¹³C NMR (151 MHz, CDCl₃) δ 162.9, 152.5, 148.2, 138.8, 131.1, 117.0, 110.4, 109.9, 108.5, 96.6, 81.8, 60.2, 45.1, 33.3, 30.6, 29.7, 13.6, 12.4, as shown in FIG. 39B. HRMS calculated for C₁₁H₁₀N₂O₂S (M+H) 448.5530 found 449.1030.

### N-(3-(2-Bromothiazol-4-yl)-2-methylcyclopent-2-en-1-yl)-5-((3aS,4S,6aR)-2-oxohexahydro-1H-thieno[3,4-d]imidazol-4-yl)pentanamide (NPb)

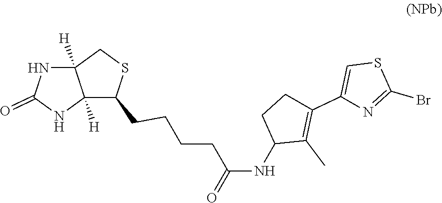 [SMILES: CC1=C(c2csc(Br)n2)CCC1NC(=O)CCCC[C@@H]1SC[C@@H]2NC(=O)N[C@@H]21]

Synthesized according to the general procedure for amide coupling using EDC-HCl with biotin-pentanoic acid (25b). (0.16 mmol, 75%). White solid; mp 120° C. ¹H NMR (600 MHz, CD₃OD) δ 11.31 (s, 1H), 8.94 (s, 1H), 8.45 (dd, J=7.7, 5.0 Hz, 1H), 8.27 (dd, J=7.8, 4.5 Hz, 1H), 7.21-7.14 (m, 1H), 6.89 (dd, J=12.7, 5.0 Hz, 1H), 6.75 (d, J=10.6 Hz, 1H), 6.67 (d, J=12.7 Hz, 1H), 6.61 (d, J=6.8 Hz, 1H), 6.36-6.28 (m, 1H), 6.23-6.17 (m, 2H), 5.99 (s, 3H), 5.76-5.54 (m, 6H), 5.44 (dd, J=15.6, 8.2 Hz, 2H), as shown in FIG. 40A. ¹³C NMR (151 MHz, CD₃OD) δ 174.4, 164.7, 153.3, 145.7, 137.8, 134.8, 129.9, 119.3, 106.5, 62.0, 61.9, 60.2, 59.8, 55.7, 55.6, 39.6, 37.9, 35.5, 32.2, 29.5, 28.4, 28.4, 28.1, 27.4, 25.6, 12.2, as shown in FIG. 40B. HRMS calculated for C₁₉H₂₅BrN₄O₂S₂ (M+H) 484.0602 found 484.0825.

### N-(3-(2-Ethynylthiazol-4-yl)-2-methylcyclopent-2-en-1-yl)-5-((3aS,4S,6aR)-2-oxohexahydro-1H-thieno[3,4-d]imidazol-4-yl)pentanamide (PPb)

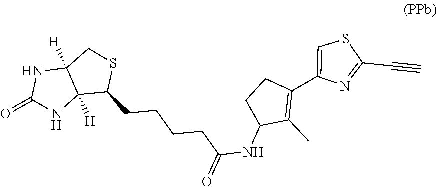 [SMILES: C#Cc1nc(C2=C(C)C(NC(=O)CCCC[C@@H]3SC[C@@H]4NC(=O)N[C@@H]43)CC2)cs1]

Synthesized according to the general procedure for Sonogashira coupling reaction with. (0.05 mmol, 55%). White solid; mp 130° C. ¹H NMR (600 MHz, CD₃OD) δ 7.47 (s, 1H), 5.01 (s, 1H), 4.51 (dd, J=7.8, 4.9 Hz, 1H), 4.33 (dd, J=7.9, 4.5 Hz, 1H), 3.36 (s, 1H), 3.26-3.20 (m, 2H), 2.95 (dd, J=12.7, 5.0 Hz, 1H), 2.85 (d, J=11.2 Hz, 1H), 2.72 (t, J=11.1 Hz, 2H), 2.43-2.36 (m, 1H), 2.30-2.23 (m, 2H), 2.04 (s, 3H), 1.81-1.60 (m, 6H), 1.54-1.46 (m, 2H), 1.31 (d, J=4.0 Hz, 1H), as shown in FIG. 41A. ¹³C NMR (151 MHz, CD₃OD) δ 174.4, 137.9, 130.4, 117.6, 82.9, 62.0, 60.2, 59.7, 55.6, 39.6, 35.5, 32.6, 29.6, 28.4, 28.1, 25.6, 12.2, as shown in FIG. 41B. HRMS calculated for C₂₁H₂₆N₄O₂S₂(M+H) 430.5850 found 449.1030.

### N-(4-(4-Aminobenzoyl)phenyl)pent-4-ynamide (33)

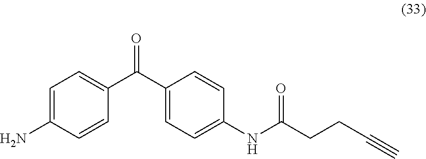 [SMILES: C#CCCC(=O)Nc1ccc(C(=O)c2ccc(N)cc2)cc1]

Synthesized according to the general procedure for amide coupling using EDC-HCl. (4.10 mmol, 87%). Cream solid. ¹H NMR (600 MHz, DMSO-d₆) δ 10.28 (s, 1H), 7.74-7.70 (m, 2H), 7.65-7.58 (m, 2H), 7.53-7.50 (m, 2H), 7.47-7.41 (m, 2H), 6.11 (s, 2H), 5.89 (s, 2H), 2.84-2.81 (m, 1H), 2.53-2.45 (m, 4H), as shown in FIG. 42A. ¹³C NMR (151 MHz, DMSO-d₆) δ 192.9, 170.3, 153.9, 152.9, 142.3, 133.8, 132.9, 132.3, 130.7, 125.9, 124.6, 118.6, 112.9, 112.8, 84.0, 72.1, 35.8, 14.5, as shown in FIG. 42B.

### 2-Bromo-N-(3-methyl-2-oxo-2H-chromen-6-yl)acetamide (28c)

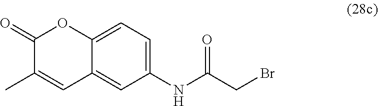 [SMILES: Cc1cc2cc(NC(=O)CBr)ccc2oc1=O]

Synthesized according to the general procedure for acylation reaction. (1.35 mmol, 47%). White solid. ¹H NMR (600 MHz, CDCl₃) δ 8.33 (s, 1H), 7.66 (d, J=2.1 Hz, 1H), 7.60 (d, J=8.6 Hz, 1H), 7.53 (dd, J=8.6, 2.1 Hz, 1H), 6.27 (d, J=1.2 Hz, 1H), 4.08 (s, 2H), 2.45 (d, J=1.2 Hz, 3H), as shown in FIG. 43A. ¹³C NMR (151 MHz, CDCl₃) δ 151.8, 140.1, 125.4, 115.6, 114.1, 107.5, 29.3, 18.6, as shown in FIG. 43B.

### N-(4-(4-(2-Bromoacetamido)benzoyl)phenyl)pent-4-ynamide (28e)

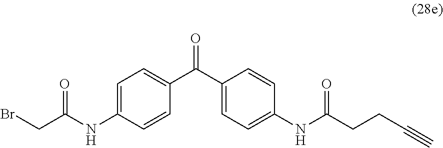 [SMILES: C#CCCC(=O)Nc1ccc(C(=O)c2ccc(NC(=O)CBr)cc2)cc1]

Synthesized according to the general procedure for acylation reaction. (0.73 mmol, 53%). ¹H NMR (600 MHz, CD₃OD) δ 7.76-7.73 (m, 8H), 4.03 (s, 2H), 2.65 (dd, J=11.2, 4.1 Hz, 2H), 2.59-2.55 (m, 2H), 2.31 (q, J=2.6 Hz, 1H), as shown in FIG. 44A. ¹³C NMR (151 MHz, CD₃OD) δ 195.0, 171.2, 166.5, 142.7, 142.3, 142.3, 133.2, 133.1, 132.5, 130.9, 130.9, 118.8, 118.7, 82.2, 69.1, 66.7, 35.6, 28.4, 14.0, as shown in FIG. 44B.

### 4-(2-Ethynylthiazol-4-yl)-2-hydroxy-3-methylcyclopent-3-en-1-one 0-prop-2-yn-1-yl oxime (PPd)

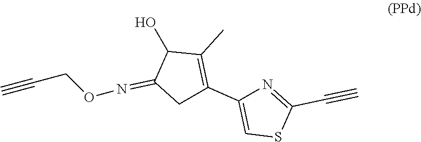 [SMILES: C#CCO/N=C1\CC(c2csc(C#C)n2)=C(C)C1O]

Synthesized according to the general procedure for alkylation of alcohol-oxime. (0.073 mmol, 43%). White solid; mp 135° C. ¹H NMR (600 MHz, CDCl₃) δ 7.21 (s, 1H), 5.08 (d, J=5.5 Hz, 1H), 4.72 (s, 2H), 3.70-3.64 (m, 1H), 3.49-3.44 (m, 2H), 2.47 (dd, J=4.1, 2.3 Hz, 1H), 2.25-2.21 (m, 3H), as shown in FIG. 45A. ¹³C NMR (151 MHz, CDCl₃) δ 162.7, 152.6, 152.5, 138.3, 127.5, 118.5, 82.3, 78.5, 76.3, 74.7, 61.7, 61.1, 36.6, 34.9, 29.7, 13.4, as shown in FIG. 45B. HRMS calculated for C₁₄H₁₂N₂O₂S (M+H) 273.220 found 273.0701.

### 4-(2-Bromothiazol-4-yl)-2-hydroxy-3-methylcyclopent-3-en-1-one O-prop-2-yn-1-yl oxime (NPd)

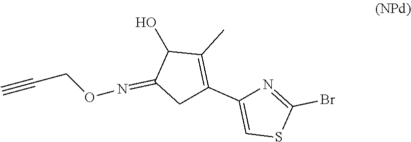 [SMILES: C#CCO/N=C1\CC(c2csc(Br)n2)=C(C)C1O]

Synthesized according to the general procedure for alkylation of alcohol-oxime. (0.062 mmol, 40%). White solid; mp 151° C. ¹H NMR (600 MHz, CDCl₃) δ 7.14 (s, 1H), 5.11 (s, 1H), 4.76 (dt, J=3.0, 1.6 Hz, 2H), 2.51 (dd, J=4.8, 2.4 Hz, 1H), 2.25 (d, J=0.8 Hz, 3H), as shown in FIG. 46A. ¹³C NMR (151 MHz, CDCl₃) δ 166.5, 162.5, 152.2, 138.3, 135.5, 127.0, 120.1, 78.5, 74.7, 61.7, 34.5, 13.3, as shown in FIG. 46B. HRMS calculated for C₁₄H₁₂N₂O₂S (M+H) 326.1960 found 326.9808.

### (E)-2-(((4-(2-Ethynylthiazol-4-yl)-3-methyl-2-oxocyclopent-3-en-1-ylidene)amino)oxy)-N-(3-methyl-2-oxo-2H-chromen-6-yl)acetamide (PPc)

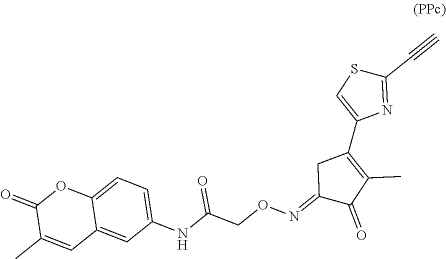 [SMILES: C#Cc1nc(C2=C(C)C(=O)/C(=N/OCC(=O)Nc3ccc4oc(=O)c(C)cc4c3)C2)cs1]

Synthesized according to the general procedure for alkylation of alcohol-oxime. (0.044 mmol, 10%); mp 145° C. ¹H NMR (600 MHz, CDCl₃) δ 8.06 (s, 1H), 7.86 (d, J=3.2 Hz, 1H), 7.62-7.54 (m, 3H), 6.24 (d, J 1.2 Hz, 1H), 4.98 (d, J 11.1 Hz, 2H), 3.89 (q, J=2.1 Hz, 2H), 3.62 (d, J=6.6 Hz, 2.44 (t, J=2.3 Hz, 3H), 2.31 (t, J=2.2 Hz, 3H), as shown in FIG. 47A. ¹³C NMR (151 MHz, CDCl₃) δ 166.8, 160.8, 154.6, 152.1, 150.4, 140.0, 125.4, 124.4, 116.8, 115.9, 113.9, 107.6, 83.9, 74.6, 31.1, 18.6, 10.7, as shown in FIG. 47B. HRMS calculated for C₂₃H₁₇N₃O₅S (M+H) 447.4650 found 447.9909.

### (E)-2-(((4-(2-Bromothiazol-4-yl)-2-hydroxy-3-methylcyclopent-3-en-1-ylidene)amino)oxy)-N-(3-methyl-2-oxo-2H-chromen-6-yl)acetamide (NPc)

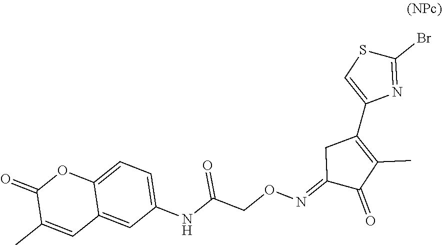 [SMILES: CC1=C(c2csc(Br)n2)C/C(=N\OCC(=O)Nc2ccc3oc(=O)c(C)cc3c2)C1=O]

Synthesized according to the general procedure for alkylation of alcohol-oxime. (0.040 mmol, 40% yield). Brown solid; mp 120° C. ¹H NMR (400 MHz, CDCl₃) δ 8.04 (s, 1H), 7.85 (s, 1H), 7.62-7.52 (m, 3H), 6.23 (s, 1H), 4.98 (s, 2H), 3.87 (s, 2H), 2.42 (s, 3H), 2.30 (s, 3H), as shown in FIG. 48A. ¹³C NMR (151 MHz, CDCl₃) δ 190.8, 166.8, 160.8, 154.6, 152.1, 152.0, 150.4, 143.5, 140.0, 125.4, 124.4, 116.7, 115.9, 113.9, 107.6, 83.9, 81.9, 74.6, 31.1, 29.7, 18.6, 10.7, as shown in FIG. 48B.

### (E)-N-(4-(4-(2-(((4-(2-Ethynylthiazol-4-yl)-2-hydroxy-3-methylcyclopent-3-en-1-ylidene)amino)oxy)acetamido)benzoyl)phenyl)pent-4-ynamide (PPe)

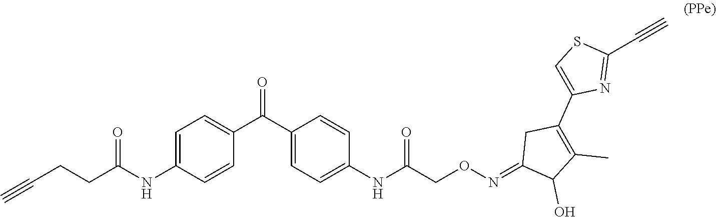 [SMILES: C#CCCC(=O)Nc1ccc(C(=O)c2ccc(NC(=O)CO/N=C3\CC(c4csc(C#C)n4)=C(C)C3O)cc2)cc1]

Synthesized according to the general procedure for alkylation of alcohol-oxime. (0.070 mmol, 20%). Cream solid; mp 160° C. ¹H NMR (600 MHz, acetone-d₆) δ 9.56 (s, 1H), 9.26 (s, 1H), 7.89-7.87 (m, 2H), 7.83 (dd, J=8.9, 2.0 Hz, 2H), 7.79-7.76 (m, 4H), 7.75 (s, 1H), 5.04 (t, J=7.3 Hz, 1H), 4.85 (d, J=7.2 Hz, 1H), 4.76-4.72 (m, 2H), 4.37 (s, 1H), 3.79-3.72 (m, 1H), 3.66-3.60 (m, 1H), 2.68 (dd, J=11.1, 3.9 Hz, 2H), 2.59-2.55 (m, 2H), 2.40 (t, J=2.7 Hz, 1H), 2.25 (d, J=0.9 Hz, 3H), as shown in FIG. 49A. ¹³C NMR (151 MHz, acetone-d₆) δ 193.3, 169.7, 168.3, 164.3, 152.8, 146.6, 143.0, 142.1, 139.2, 133.2, 132.5, 130.9, 130.8, 126.4, 119.3, 118.9, 118.9, 118.3, 118.2, 83.5, 82.9, 77.9, 77.9, 77.8, 76.4, 73.3, 69.5, 35.8, 35.7, 34.6, 13.9, 12.7, as shown in FIG. 49B. HRMS calculated for C₃₁H₂₆N₄O₅S (M+H) 566.6320 found 566.2771.

### (E)-N-(4-(4-(2-(((4-(2-Bromothiazol-4-yl)-2-hydroxy-3-methylcyclopent-3-en-1-ylidene)amino)oxy)acetamido)benzoyl)phenyl)pent-4-ynamide (NPe)

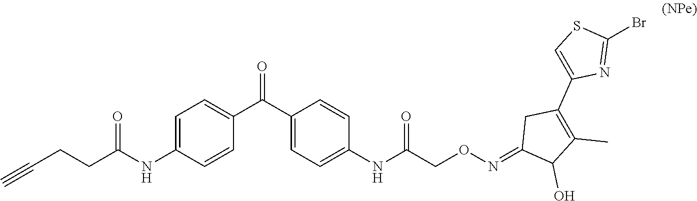 [SMILES: C#CCCC(=O)Nc1ccc(C(=O)c2ccc(NC(=O)CO/N=C3\CC(c4csc(Br)n4)=C(C)C3O)cc2)cc1]

Synthesized according to the general procedure for alkylation of alcohol-oxime. (0.08 mmol, 22%). Cream solid; mp 170° C. ¹H NMR (600 MHz, acetone-d₆) δ 9.57 (s, 1H), 9.27 (s, 1H), 7.85 (dt, J=23.4, 11.8 Hz, 5H), 7.78 (d, J=8.7 Hz, 4H), 7.68 (s, 1H), 5.04 (d, J=7.3 Hz, 1H), 4.86 (d, J=7.2 Hz, 1H), 4.73 (d, J=2.9 Hz, 2H), 3.72 (d, J=15.6 Hz, 1H), 3.59 (d, J=15.6 Hz, 1H), 2.68 (t, J=7.2 Hz, 2H), 2.57 (t, J=6.9 Hz, 2H), 2.40 (dd, J=3.4, 1.6 Hz, 1H), 2.23 (s, 3H), as shown in FIG. 50A. ¹³C NMR (151 MHz, acetone-d₆) δ 205.3, 193.3, 169.7, 168.3, 164.1, 152.4, 143.0, 142.1, 139.2, 135.0, 133.2, 132.5, 130.9, 130.8, 125.9, 121.2, 118.9, 118.9, 118.2, 82.9, 77.8, 77.7, 73.3, 69.5, 35.8, 34.3, 13.9, 12.6, as shown in FIG. 50B. HRMS calculated for C₂₉H₂₅BrN₄O₅S (M+H) 621.5060 found 621.7080.

General Procedure for Boc-Protection of Amines with Di-Tert-Butyl Decarbonate

The amine (1.0 equiv.), di-tert-butyl decarbonate (1.1 equiv.), and 4-dimethylaminopyridine (10 mol %) were dissolved in anhydrous dichloromethane (3 mL) in an oven-dried round bottom flask equipped with a magnetic stir bar. The resulting mixture was stirred overnight at room temperature. On completion, the reaction mixture was diluted with brine (5 mL) and extracted with dichloromethane (3×3 mL). The combined organic phase was dried over anhydrous sodium sulfate, filtered, and concentrated under reduced pressure. The crude product was purified by flash column chromatography on silica gel in 0 to 10% ethyl acetate in hexanes to obtain a pure product of interest.

### Methyl 5-bromo-2-((tert-butoxycarbonyl)amino)benzoate (35)

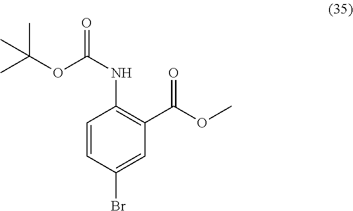 [SMILES: COC(=O)c1cc(Br)ccc1NC(=O)OC(C)(C)C]

Synthesized according to the general procedure for Boc-protection of amines. (7.57 mmol, 88%). White solid. ¹H NMR (600 MHz, CDCl₃) δ 10.46 (s, 1H), 8.39 (d, J=9.1 Hz, 1H), 8.15 (d, J=2.5 Hz, 1H), 7.64 (dd, J=9.1, 2.4 Hz, 1H), 3.81 (s, 3H), 1.59 (s, 9H), as shown in FIG. 51A. ¹³C NMR (151 MHz, CDCl₃) δ 167.4, 153.9, 140.8, 137.3, 133.4, 120.6, 116.1, 113.9, 52.6, 52.5, 27.4, as shown in FIG. 51B.

### Methyl 5-azido-2-((tert-butoxycarbonyl)amino)benzoate (36)

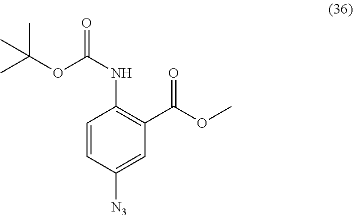 [SMILES: COC(=O)c1cc(N=[N+]=[N-])ccc1NC(=O)OC(C)(C)C]

Methyl 5-bromo-2-((tert-butoxycarbonyl)amino)benzoate (35) (110 mg, 0.335 mmol, 1 equiv.), copper iodide (21.15 mg, 0.066 mmol, 2 mol %), sodium ascorbate (6.6 mg, 0.033 mmol, 0.1 equiv.), sodium azide (1.1 equiv.), and 1,2-dimethylethylenediamine (8.8 mg, 0.017 mL, 0.099 mmol, 0.3 equiv.) were dissolved in ethanol/water (2:1) (3 mL) in a round bottom flask equipped with a magnetic stir bar. The reaction mixture was heated under reflux overnight whilst monitored by TLC. On completion, ethanol was removed under reduced pressure before diluting the mixture with brine (5 mL) and extracted with ethyl acetate (3×5 mL). The combined organic phase was dried over anhydrous sodium sulfate, filtered, and concentrated under reduced pressure. The crude product was purified by flash chromatography on silica gel in 0 to 40% ethyl acetate in hexanes to obtain a pure methyl 5-azido-2-((tert-butoxycarbonyl)amino)benzoate (36) (0.328 mmol, 98%) as a white solid. ¹H NMR (600 MHz, CDCl₃) δ 10.19 (d, J=16.5 Hz, 1H), 8.46 (t, J=13.5 Hz, 1H), 7.66 (d, J=2.6 Hz, 1H), 7.18 (dd, J=9.1, 2.5 Hz, 1H), 3.93 (d, J=8.9 Hz, 2H), 1.55 (d, J=15.3 Hz, 9H), as shown in FIG. 52A. ¹³C NMR (151 MHz, CDCl₃) δ 167.7, 139.5, 132.9, 125.1, 120.8, 120.5, 115.4, 91.3, 85.6, 28.3, as shown in FIG. 52B.

### 5-Azido-2-((tert-butoxycarbonyl)amino)benzoic acid (37)

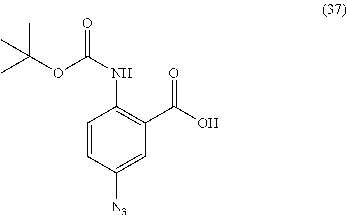 [SMILES: CC(C)(C)OC(=O)Nc1ccc(N=[N+]=[N-])cc1C(=O)O]

Ester (36) (400 mg, 1.37 mmol, 1 equiv.) was dissolved in methanol/water (3:1) (3 mL). Potassium carbonate (580 mg, 4.2 mmol, 3 equiv.) was added and the mixture was stirred for 12 hours at room temperature. On completion, the reaction mixture was acidified with 5% hydrochloric acid. The product precipitated was collected by filtration, washed with ice water, and dried under vacuum to yield a pure 5-azido-2-((tert-butoxycarbonyl)amino)benzoic acid 37 (1.37 mmol, 98%) as a white solid. ¹H NMR (400 MHz, CDCl₃) δ 9.93 (s, 1H), 8.51 (d, J=9.1 Hz, 1H), 7.72 (s, 1H), 7.22 (d, J=9.0 Hz, 1H), 1.53 (dd, J=10.8, 8.9 Hz, 9H), as shown in FIG. 53A. ¹³C NMR (151 MHz, CDCl₃), as shown in FIG. 53B.

### Tert-butyl (4-azido-2-(prop-2-yn-1-ylcarbamoyl)phenyl)carbamate (39)

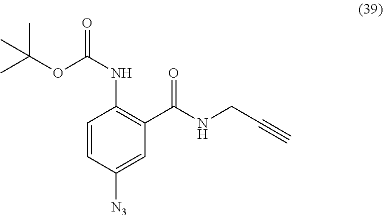 [SMILES: C#CCNC(=O)c1cc(N=[N+]=[N-])ccc1NC(=O)OC(C)(C)C]

Synthesized according to the general for amide coupling using EDC-HCl. (1.27 mmol, 70%). White solid. ¹H NMR (600 MHz, CDCl₃) δ 9.85 (s, 1H), 8.41 (d, J=9.0 Hz, 1H), 7.16 (dd, J=9.0, 2.6 Hz, 1H), 7.05 (d, J=2.6 Hz, 1H), 6.35 (s, 1H), 4.25 (dd, J=5.1, 2.5 Hz, 2H), 2.35 (t, J=2.5 Hz, 1H), 1.53 (s, 9H), as shown in FIG. 54A. ¹³C NMR (151 MHz, CDCl₃) δ 167.6, 152.9, 137.36, 133.3, 122.9, 121.7, 120.4, 117.1, 80.7, 78.7, 72.5, 29.8, 28.3, as shown in FIG. 54B.

### 2-Amino-5-azido-N-(prop-2-yn-1-yl)benzamide (40)

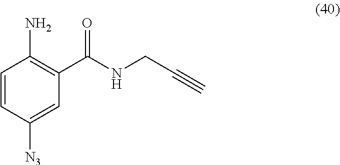 [SMILES: C#CCNC(=O)c1cc(N=[N+]=[N-])ccc1N]

The carbamate 37 (1.0 equiv.) was dissolved in DCM/TFA (1:1) (4 mL) at 0° C. in an oven-dried round bottom flask equipped with a magnetic stir bar. The reaction mixture was allowed to warm up to room temperature and maintained at this temperature until completion. Aqueous sodium bicarbonate (5 mL) was added and extracted with dichloromethane (3×5 mL). The combined organic phase was dried over anhydrous sodium sulfate, filtered, and concentrated under reduced pressure to yield 2-amino-5-azido-N-(prop-2-yn-1-yl)benzamide 40 (1.16 mmol, 91.57%) as an oil. ¹H NMR (600 MHz, CDCl₃) δ 7.01-6.96 (m, 1H), 6.75-6.71 (m, 1H), 6.18 (s, 1H), 5.47 (s, 1H), 4.28-4.19 (m, 1H), 2.32 (t, J=2.5 Hz, 1H), as shown in FIG. 55A. ¹³C NMR (151 MHz, CDCl₃) δ 167.9, 146.2, 128.5, 123.6, 118.9, 117.5, 115.9, 72.1, 72.0, 29.6, as shown in FIG. 55B.

### 5-Azido-2-(2-bromoacetamido)-N-(prop-2-yn-1-yl)benzamide (28f)

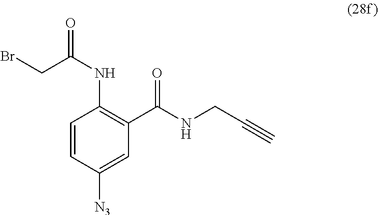 [SMILES: C#CCNC(=O)c1cc(N=[N+]=[N-])ccc1NC(=O)CBr]

Synthesized according to general procedure for acylation. (0.65 mmol, 46%). White solid. ¹H NMR (600 MHz, CDCl₃) δ 11.43 (s, 1H), 8.62 (dd, J=9.0, 2.0 Hz, 1H), 7.24 (dd, J=5.7, 3.2 Hz, 1H), 7.14-7.08 (m, 1H), 6.35 (s, 1H), 4.28 (dt, J=4.4, 2.3 Hz, 2H), 4.03-3.97 (m, 2H), 2.40-2.31 (m, 1H), as shown in FIG. 56A. ¹³C NMR (151 MHz, CDCl₃) δ 167.3, 164.8, 135.7, 135.6, 123.4, 122.9, 121.9, 117.2, 78.5, 72.8, 30.0, 29.5, as shown in FIG. 56B.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A composition comprising Formula I: 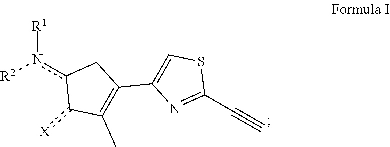
wherein:
dashed lines represent optional bonds, provided that nitrogen does not have more than three bonds and oxygen does not have more than two bonds;
R¹ is OH, acyl, aryl, alkoxy, alkoxyalkyl, heteroaryl, aralkyl, or amidyl;
R² is absent or hydrogen; and
X is absent, OH, or OR³, wherein R³ is absent, hydrogen, alkyl, alkoxy, or alkoxyalkyl;
or a stereoisomer, racemate, solvate, hydrate, polymorph, or prodrug thereof.

2. The composition of claim 1, wherein R² is absent and X is OH.

3. The composition of claim 1, wherein R² is hydrogen and X is OH.

4. The composition of claim 1, wherein R² is absent and X is absent.

5. The composition of claim 1, wherein R² is absent, X is OR³, and R³ is alkyl, alkoxy, or alkoxyalkyl.

6. The composition of claim 1, wherein R² is hydrogen, X is OR³, and R³ is alkyl, alkoxy, or alkoxyalkyl.

7. The composition of claim 1, wherein R¹ is an ester with a terminal alkyne.

8. The composition of claim 1, wherein R¹ is OR⁴, wherein R⁴ is aryl, aralkyl, or amidyl.

9. The composition of claim 1, wherein R¹ is OC═OR⁵, wherein R⁵ is alkyl.

10. The composition of claim 9, wherein R⁵ is (CH₂)ₙCH₃, wherein n ranges from 1 to 10.

11. The composition of claim 1, comprising any of compounds 14, 19, 18, 20a, 20b, 20c, 20e, 21, 23, PPc, PPa, PPb, PPd, PPe, or PPf:

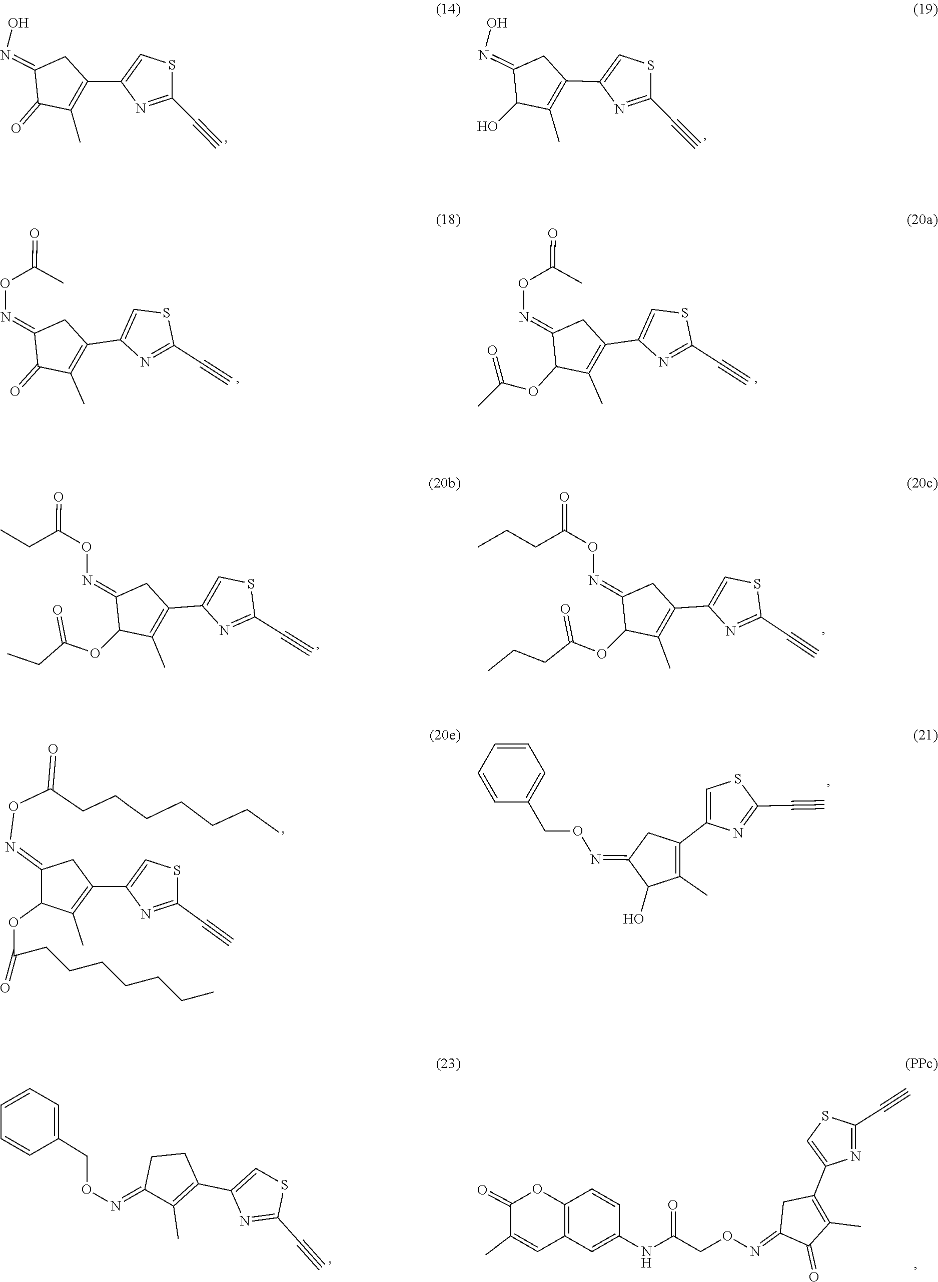

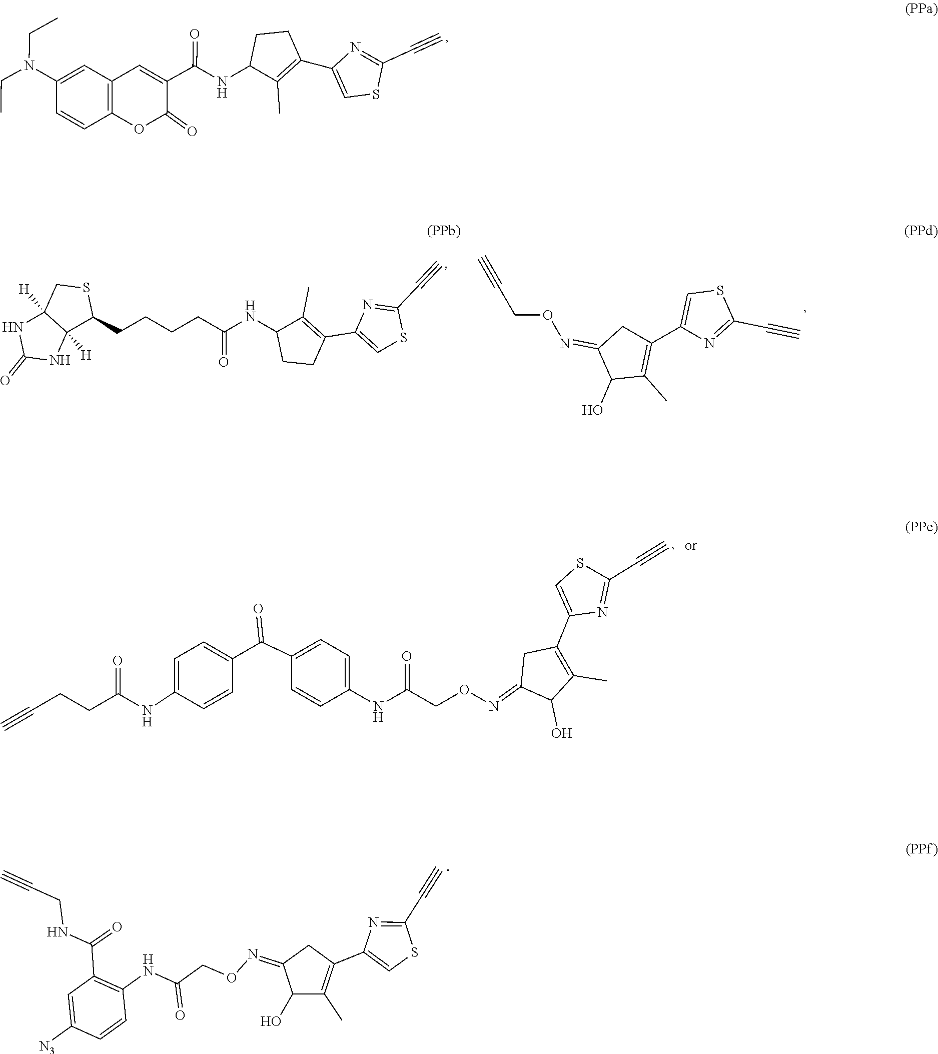

12. A method to kill cancer cells, the method comprising contacting cancer cells with an effective amount of a composition of claim 1 to kill the cancer cells.

13. The method of claim 12, wherein the cancer cells are mesenchymal non-small cell lung cancer cells, fibrosarcoma cells, osteosarcoma cells, or breast cancer cells.

14. The method of claim 12, wherein the composition comprises compound 20a 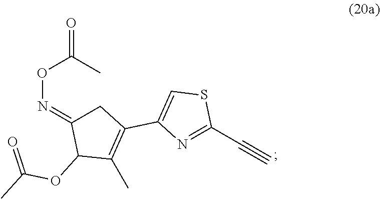 [SMILES: C#Cc1nc(C2=C(C)C(OC(C)=O)/C(=N/OC(C)=O)C2)cs1]
and the cancer cells comprise leukemia cells, ovarian cancer cells, or renal cancer cells.

15. A method of treating a cancer, the method comprising administering to a subject having a cancer an effective amount of a composition of claim 1 to treat the cancer.

16. The method of claim 15, wherein the cancer is non-small cell lung cancer or breast cancer.

17. The method of claim 15, wherein the composition comprises compound 20a 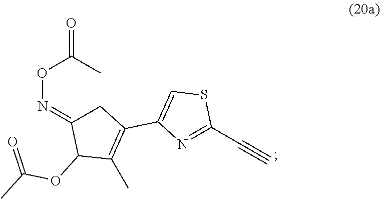 [SMILES: C#Cc1nc(C2=C(C)C(OC(C)=O)/C(=N/OC(C)=O)C2)cs1]
and the cancer is leukemia, ovarian cancer, or renal cancer.

18. A method of inhibiting tumor metastasis in a subject, the method comprising administering an effective amount of a composition of claim 1 to a subject having a tumor to inhibit tumor metastasis in the subject.

19. A method of making a CETZOLE compound, the method comprising:
condensing an ethyl vinyl ketone with a thiazole aldehyde in the presence of a thiazolium salt catalyst to obtain a 1,4-addition product;
cyclizing the 1,4-addition product to obtain a halo-ketone;
reducing the halo-ketone to obtain an alcohol;
coupling the alcohol with trimethylsilyl-acetylene to obtain a silylated ketone;
desilylating the silylated ketone to obtain a ketone; and
reducing the ketone to obtain a CETZOLE compound.

20. The method of claim 19, wherein the CETZOLE compound is obtained in a racemic mixture.
